SEQUENCE LISTING (59 sequences)

SEQ ID NO: 1 - protein, 5 aa, Artificial Sequence
  SITE 1: X is D or N
  CDR1 VH
XHAMA

SEQ ID NO: 2 - protein, 17 aa, Artificial Sequence
  CDR2 VH
YISYDGDNTYYRDSVKG

SEQ ID NO: 3 - protein, 7 aa, Artificial Sequence
  CDR3 VH
GGNSGYD

SEQ ID NO: 4 - protein, 11 aa, Artificial Sequence
  CDR1 VL
  SITE 2: X is A or G
  SITE 10: X is L or V
KXSQNVNKFXN

SEQ ID NO: 5 - protein, 7 aa, Artificial Sequence
  CDR2 VL
GTNSLQT

SEQ ID NO: 6 - protein, 9 aa, Artificial Sequence
  CDR3 VL
  SITE 4: X is S or T
QQYXSWPWT

SEQ ID NO: 7 - protein, 5 aa, Artificial Sequence
  CDR1 VH
DHAMA

SEQ ID NO: 8 - protein, 11 aa, Artificial Sequence
  CDR1 VL
KASQNVNKFLN

SEQ ID NO: 9 - protein, 9 aa, Artificial Sequence
  CDR3 VL
QQYSSWPWT

SEQ ID NO: 10 - protein, 5 aa, Artificial Sequence
  CDR1 VH
NHAMA

SEQ ID NO: 11 - protein, 11 aa, Artificial Sequence
  CDR1 VL
KASQNVNKFVN

SEQ ID NO: 12 - protein, 11 aa, Artificial Sequence
  CDR1 VL
KGSQNVNKFLN

SEQ ID NO: 13 - protein, 9 aa, Artificial Sequence
  CDR3 VL
QQYTSWPWT

SEQ ID NO: 14 - protein, 116 aa, Artificial Sequence
  VH
EVQLVESGGGLVQPGRSMKLSCAASGFPFSDHAMAWVRQAPKKGLQWVAYISYDGDNTYYRDSVKGRFTISRDNARSTLFLQMDSLRSEDTATYYCTTGGNSGYDWGQGVMVTVSS

SEQ ID NO: 15 - protein, 116 aa, Artificial Sequence
  VH
EVQLVESGGGLVQPGRSMKLSCAASGFTFSNHAMAWVRQAPKKGLEWVAYISYDGDNTYYRDSVKGRFTISRDNAKSTLYLQIDSLRSEDTATYYCTTGGNSGYDWGQGVMVTVSS

SEQ ID NO: 16 - protein, 116 aa, Artificial Sequence
  VH
EVQLVESGGGLVQPGRSMKLSCAVSGFTFSNHAMAWVRQAPKKGLEWVAYISYDGDNTYYRDSVKGRFTISRDNAQSTLYLQMDSLRSEDTATYYCTTGGNSGYDWGQGVMVTVSS

SEQ ID NO: 17 - protein, 107 aa, Artificial Sequence
  VL
DIQMTQSPSFLSASVGDRVTINCKASQNVNKFLNWYQQKLGEAPRRLIYGTNSLQTGIPSRFSGSGSGTDYTLTISSLQPEDVATYFCQQYSSWPWTFGGGTKLELK

SEQ ID NO: 18 - protein, 107 aa, Artificial Sequence
  VL
DIQMTQSPSFLSASVGDRVTINCKASQNVNKFVNWYQQKLGEAPRRLIYGTNSLQTGIPSRFSGSGSGTDYTLTISSLQPEDVATYFCQQYSSWPWTFGGGTKLELK

SEQ ID NO: 19 - protein, 107 aa, Artificial Sequence
  VL
DIQMTQSPSFLSASVGDRVTINCKGSQNVNKFLNWYQQKLGEAPRRLIYGTNSLQTGIPSRFSGSGSGTDYTLTISSLQPEDVATYFCQQYTSWPWTFGGGTKLELK

SEQ ID NO: 20 - protein, 116 aa, Artificial Sequence
  VH
  SITE 24: X is A or V
  SITE 28: X is P or T
  SITE 31: X is D or N
  SITE 46: X is Q or E
  SITE 76: X is R, K or Q
  SITE 80: X is F or Y
  SITE 83: X is M or I
EVQLVESGGGLVQPGRSMKLSCAXSGFXFSXHAMAWVRQAPKKGLXWVAYISYDGDNTYYRDSVKGRFTISRDNAXSTLXLQXDSLRSEDTATYYCTTGGNSGYDWGQGVMVTVSS

SEQ ID NO: 21 - protein, 107 aa, Artificial Sequence
  VL
  SITE 25: X is A or G
  SITE 33: X is L or V
  SITE 92: X is S or T
DIQMTQSPSFLSASVGDRVTINCKXSQNVNKFXNWYQQKLGEAPRRLIYGTNSLQTGIPSRFSGSGSGTDYTLTISSLQPEDVATYFCQQYXSWPWTFGGGTKLELK

SEQ ID NO: 22 - protein, 19 aa, Artificial Sequence
  leader sequence
MDIRLSLAFLVLFIKGVQC

SEQ ID NO: 23 - protein, 19 aa, Artificial Sequence
  leader sequence
MAAVQLLGLLLLWLPAMRC

SEQ ID NO: 24 - DNA, 347 bp, Artificial Sequence
  VH
  misc_feature 18: /note=“n is G or A”
  misc_feature 71: /note=“n is C or T”
  misc_feature 82: /note=“n is C or A”
  misc_feature 91: /note=“n is G or A”
  misc_feature 136: /note=“n is C or G”
  misc_feature 225: /note=“n is A or C”
  misc_feature 226: /note=“n is G or A”
  misc_feature 230: /note=“n is T or C”
  misc_feature 238: /note=“n is T or A”
  misc_feature 248: /note=“n is G or T”
  misc_feature 284: /note=“n is C or T”
gaggtgcagctggtggantctgggggcggcttagtgcagcctggaaggtccatgaaactctcctgtgcagnctcaggattcnctttcagtnaccatgccatggcctgggtccgccaggctccaaagaagggtctgnagtgggtcgcatacattagttatgatgggataacacttactatcgagactccgtgaagggccgattcactatctccagagataatgcannaagnaccctatncctgcaaatngacagtctgaggtctgaggacacggccacttattantgtacaacagggggtaattcggggtacgactggggccaaggagtcatggtcacagtctcctca

SEQ ID NO: 25 - DNA, 321 bp, Artificial Sequence
  VL
  misc_feature 60: /note=“n is C or T”
  misc_feature 74: /note=“n is C or G”
  misc_feature 97: /note=“n is T or G”
  misc_feature 204: /note=“n is T or A”
  misc_feature 275: /note=“n is G or C”
gacatccagatgacccagtctccttcattcctgtctgcatctgtgggagacagagtcacnatcaactgcaaagnaagtcagaatgttaacaagttcntaaactggtatcagcaaaagcttggagaagctcccagacgcctgatctatggtacaaacagtttgcaaaccggcatcccatcaaggttcagtggcagtggatctggnacagattacacactcaccatcagcagcctgcagcctgaagatgttgccacgtatttctgccagcagtatancagttggccgtggacgttcggtggaggcaccaagctggaattgaaa

SEQ ID NO: 26 - DNA, 348 bp, Artificial Sequence
  VH
gaggtgcagctggtggagtctgggggcggcttagtgcagcctggaaggtccatgaaactctcctgtgcagcctcaggattccctttcagtgaccatgccatggcctgggtccgccaggctccaaagaagggtctgcagtgggtcgcatacattagttatgatggggataacacttactatcgagactccgtgaagggccgattcactatctccagagataatgcaagaagtaccctattcctgcaaatggacagtctgaggtctgaggacacggccacttattattgtacaacagggggtaattcggggtacgactggggccaaggagtcatggtcacagtctcctca

SEQ ID NO: 27 - DNA, 348 bp, Artificial Sequence
  VH
gaggtgcagctggtggaatctgggggcggcttagtgcagcctggaaggtccatgaaactctcctgtgcagcctcaggattcactttcagtaaccatgccatggcctgggtccgccaggctccaaagaagggtctggagtgggtcgcatacattagttatgatggggataacacttactatcgagactccgtgaagggccgattcactatctccagagataatgcaaaaagcaccctatacctgcaaattgacagtctgaggtctgaggacacggccacttattactgtacaacagggggtaattcggggtacgactggggccaaggagtcatggtcacagtctcctca

SEQ ID NO: 28 - DNA, 348 bp, Artificial Sequence
  VH
gaggtgcagctggtggagtctgggggcggcttagtgcagcctggaaggtccatgaaactctcctgtgcagtctcaggattcactttcagtaaccatgccatggcctgggtccgccaggctccaaagaagggtctggagtgggtcgcatacattagttatgatggggataacacttactatcgagactccgtgaagggccgattcactatctccagagataatgcacaaagcaccctatacctgcaaatggacagtctgaggtctgaggacacggccacttattactgtacaacagggggtaattcggggtacgactggggccaaggagtcatggtcacagtctcctca

SEQ ID NO: 29 - DNA, 321 bp, Artificial Sequence
  VL
gacatccagatgacccagtctccttcattcctgtctgcatctgtgggagacagagtcaccatcaactgcaaagcaagtcagaatgttaacaagttcttaaactggtatcagcaaaagcttggagaagctcccagacgcctgatctatggtacaaacagtttgcaaaccggcatcccatcaaggttcagtggcagtggatctggtacagattacacactcaccatcagcagcctgcagcctgaagatgttgccacgtatttctgccagcagtatagcagttggccgtggacgttcggtggaggcaccaagctggaattgaaa

SEQ ID NO: 30 - DNA, 321 bp, Artificial Sequence
  VL
gacatccagatgacccagtctccttcattcctgtctgcatctgtgggagacagagtcactatcaactgcaaagcaagtcagaatgttaacaagttcgtaaactggtatcagcaaaagcttggagaagctcccagacgcctgatctatggtacaaacagtttgcaaaccggcatcccatcaaggttcagtggcagtggatctggaacagattacacactcaccatcagcagcctgcagcctgaagatgttgccacgtatttctgccagcagtatagcagttggccgtggacgttcggtggaggcaccaagctggaattgaaa

SEQ ID NO: 31 - DNA, 321 bp, Artificial Sequence
  VL
gacatccagatgacccagtctccttcattcctgtctgcatctgtgggagacagagtcactatcaactgcaaaggaagtcagaatgttaacaagttcttaaactggtatcagcaaaagcttggagaagctcccagacgcctgatctatggtacaaacagtttgcaaaccggcatcccatcaaggttcagtggcagtggatctggtacagattacacactcaccatcagcagcctgcagcctgaagatgttgccacgtatttctgccagcagtataccagttggccgtggacgttcggtggaggcaccaagctggaattgaaa

SEQ ID NO: 32 - DNA, 57 bp, Artificial Sequence
  leader sequence
atggacatcaggctcagcttggctttccttgtccttttcataaaaggtgtccagtgt

SEQ ID NO: 33 - DNA, 57 bp, Artificial Sequence
  leader sequence
atggctgcagttcaactcttagggctgctgctgctttggctcccagccatgagatgt

SEQ ID NO: 34 - protein, 11 aa, Artificial Sequence
  CDR1 VL
KGSQNVNKFVN

SEQ ID NO: 35 - protein, 116 aa, Artificial Sequence
  SITE 1: X is E or Q
  VH
  SITE 11: X is L or V
  SITE 18: X is M or L
  SITE 19: X is K or R
  SITE 24: X is A or V
  SITE 28: X is T or P
  SITE 31: X is N or D
  SITE 42: X is K or G
  SITE 46: X is E or Q
  SITE 75: X is A or S
  SITE 76: X is K, R or Q
  SITE 77: X is S or N
  SITE 80: X is Y or F
  SITE 83: X is I or M
  SITE 84: X is D or N
  SITE 88: X is S or A
  SITE 93: X is T or V
  SITE 110: X is V or T
  SITE 111: X is M or L
XVQLVESGGGXVQPGRSXXLSCAXSGFXFSXHAMAWVRQAPXKGLXWVAYISYDGDNTYYRDSVKGRFTISRDNXXXTLXLQXXSLRXEDTAXYYCTTGGNSGYDWGQGXXVTVSS

SEQ ID NO: 36 - protein, 107 aa, Artificial Sequence
  VL
  SITE 10: X is F or S
  SITE 22: X is N or T
  SITE 25: X is A or G
  SITE 33: X is V or L
  SITE 40: X is L or P
  SITE 42: X is E or K
  SITE 45: X is R or K
  SITE 46: X is R or L
  SITE 58: X is I or V
  SITE 71: X is Y or F
  SITE 83: X is V or F
  SITE 87: X is F or Y
  SITE 92: X is S or T
  SITE 100: X is G or Q
  SITE 106: X is L or I
DIQMTQSPSXLSASVGDRVTIXCKXSQNVNKFXNWYQQKXGXAPXXLIYGTNSLQTGXPSRFSGSGSGTDXTLTISSLQPEDXATYXCQQYXSWPWTFGXGTKLEXK

SEQ ID NO: 37 - protein, 116 aa, Artificial Sequence
  VH
QVQLVESGGGVVQPGRSLRLSCAASGFTFSNHAMAWVRQAPKKGLEWVAYISYDGDNTYYRDSVKGRFTISRDNAQSTLYLQMNSLRAEDTAVYYCTTGGNSGYDWGQGTLVTVSS

SEQ ID NO: 38 - protein, 116 aa, Artificial Sequence
  VH
QVQLVESGGGVVQPGRSLRLSCAASGFTFSDHAMAWVRQAPKKGLEWVAYISYDGDNTYYRDSVKGRFTISRDNAQSTLYLQMNSLRAEDTAVYYCTTGGNSGYDWGQGTLVTVSS

SEQ ID NO: 39 - protein, 116 aa, Artificial Sequence
  VH
QVQLVESGGGVVQPGRSLRLSCAASGFTFSNHAMAWVRQAPGKGLEWVAYISYDGDNTYYRDSVKGRFTISRDNAKSTLYLQMNSLRAEDTAVYYCTTGGNSGYDWGQGTLVTVSS

SEQ ID NO: 40 - protein, 116 aa, Artificial Sequence
  VH
QVQLVESGGGVVQPGRSLRLSCAASGFTFSDHAMAWVRQAPGKGLEWVAYISYDGDNTYYRDSVKGRFTISRDNAKSTLYLQMNSLRAEDTAVYYCTTGGNSGYDWGQGTLVTVSS

SEQ ID NO: 41 - protein, 116 aa, Artificial Sequence
  VH
EVQLVESGGGLVQPGRSMKLSCAASGFPFSNHAMAWVRQAPKKGLQWVAYISYDGDNTYYRDSVKGRFTISRDNARSTLFLQMDSLRSEDTATYYCTTGGNSGYDWGQGVMVTVSS

SEQ ID NO: 42 - protein, 116 aa, Artificial Sequence
  VH
EVQLVESGGGLVQPGRSMKLSCAASGFTFSDHAMAWVRQAPKKGLEWVAYISYDGDNTYYRDSVKGRFTISRDNAKSTLYLQIDSLRSEDTATYYCTTGGNSGYDWGQGVMVTVSS

SEQ ID NO: 43 - protein, 116 aa, Artificial Sequence
  VH
EVQLVESGGGLVQPGRSMKLSCAVSGFTFSDHAMAWVRQAPKKGLEWVAYISYDGDNTYYRDSVKGRFTISRDNAQSTLYLQMDSLRSEDTATYYCTTGGNSGYDWGQGVMVTVSS

SEQ ID NO: 44 - protein, 116 aa, Artificial Sequence
  VH
QVQLVESGGGVVQPGRSLRLSCAASGFTFSDHAMAWVRQAPGKGLEWVAYISYDGDNTYYRDSVKGRFTISRDNSKSTLYLQMNSLRAEDTAVYYCTTGGNSGYDWGQGTLVTVSS

SEQ ID NO: 45 - protein, 116 aa, Artificial Sequence
  VH
QVQLVESGGGVVQPGRSLRLSCAASGFTFSNHAMAWVRQAPGKGLEWVAYISYDGDNTYYRDSVKGRFTISRDNSKSTLYLQMNSLRAEDTAVYYCTTGGNSGYDWGQGTLVTVSS

SEQ ID NO: 46 - protein, 116 aa, Artificial Sequence
  VH
QVQLVESGGGVVQPGRSLRLSCAASGFTFSDHAMAWVRQAPGKGLEWVAYISYDGDNTYYRDSVKGRFTISRDNSKNTLYLQMNSLRAEDTAVYYCTTGGNSGYDWGQGTLVTVSS

SEQ ID NO: 47 - protein, 116 aa, Artificial Sequence
  VH
QVQLVESGGGVVQPGRSLRLSCAASGFTFSNHAMAWVRQAPGKGLEWVAYISYDGDNTYYRDSVKGRFTISRDNSKNTLYLQMNSLRAEDTAVYYCTTGGNSGYDWGQGTLVTVSS

SEQ ID NO: 48 - protein, 107 aa, Artificial Sequence
  VL
DIQMTQSPSSLSASVGDRVTITCKGSQNVNKFLNWYQQKLGEAPRRLIYGTNSLQTGVPSRFSGSGSGTDYTLTISSLQPEDFATYYCQQYTSWPWTFGQGTKLEIK

SEQ ID NO: 49 - protein, 107 aa, Artificial Sequence
  VL
DIQMTQSPSSLSASVGDRVTITCKASQNVNKFLNWYQQKLGEAPRRLIYGTNSLQTGVPSRFSGSGSGTDYTLTISSLQPEDFATYYCQQYSSWPWTFGQGTKLEIK

SEQ ID NO: 50 - protein, 107 aa, Artificial Sequence
  VL
DIQMTQSPSSLSASVGDRVTITCKASQNVNKFVNWYQQKLGEAPRRLIYGTNSLQTGVPSRFSGSGSGTDYTLTISSLQPEDFATYYCQQYSSWPWTFGQGTKLEIK

SEQ ID NO: 51 - protein, 107 aa, Artificial Sequence
  VL
DIQMTQSPSSLSASVGDRVTITCKASQNVNKFLNWYQQKPGKAPRRLIYGTNSLQTGVPSRFSGSGSGTDYTLTISSLQPEDFATYYCQQYSSWPWTFGQGTKLEIK

SEQ ID NO: 52 - protein, 107 aa, Artificial Sequence
  VL
DIQMTQSPSSLSASVGDRVTITCKASQNVNKFVNWYQQKPGKAPRRLIYGTNSLQTGVPSRFSGSGSGTDYTLTISSLQPEDFATYYCQQYSSWPWTFGQGTKLEIK

SEQ ID NO: 53 - protein, 107 aa, Artificial Sequence
  VL
DIQMTQSPSSLSASVGDRVTITCKGSQNVNKFLNWYQQKPGKAPRRLIYGTNSLQTGVPSRFSGSGSGTDYTLTISSLQPEDFATYYCQQYTSWPWTFGQGTKLEIK

SEQ ID NO: 54 - protein, 107 aa, Artificial Sequence
  VL
DIQMTQSPSSLSASVGDRVTITCKASQNVNKFLNWYQQKPGKAPRRLIYGTNSLQTGVPSRFSGSGSGTDFTLTISSLQPEDFATYYCQQYSSWPWTFGQGTKLEIK

SEQ ID NO: 55 - protein, 107 aa, Artificial Sequence
  VL
DIQMTQSPSSLSASVGDRVTITCKASQNVNKFVNWYQQKPGKAPRRLIYGTNSLQTGVPSRFSGSGSGTDFTLTISSLQPEDFATYYCQQYSSWPWTFGQGTKLEIK

SEQ ID NO: 56 - protein, 107 aa, Artificial Sequence
  VL
DIQMTQSPSSLSASVGDRVTITCKGSQNVNKFLNWYQQKPGKAPRRLIYGTNSLQTGVPSRFSGSGSGTDFTLTISSLQPEDFATYYCQQYTSWPWTFGQGTKLEIK

SEQ ID NO: 57 - protein, 107 aa, Artificial Sequence
  VL
DIQMTQSPSSLSASVGDRVTITCKASQNVNKFLNWYQQKPGKAPKLLIYGTNSLQTGVPSRFSGSGSGTDFTLTISSLQPEDFATYYCQQYSSWPWTFGQGTKLEIK

SEQ ID NO: 58 - protein, 107 aa, Artificial Sequence
  VL
DIQMTQSPSSLSASVGDRVTITCKASQNVNKFVNWYQQKPGKAPKLLIYGTNSLQTGVPSRFSGSGSGTDFTLTISSLQPEDFATYYCQQYSSWPWTFGQGTKLEIK

SEQ ID NO: 59 - protein, 107 aa, Artificial Sequence
  VL
DIQMTQSPSSLSASVGDRVTITCKGSQNVNKFLNWYQQKPGKAPKLLIYGTNSLQTGVPSRFSGSGSGTDFTLTISSLQPEDFATYYCQQYTSWPWTFGQGTKLEIK

(19) United States
(12) Patent Application Publication
(10) Pub. No.: US 2022/0251232 A1
(43) Pub. Date: Aug. 11, 2022

(54) NOVEL ANTI-CD25 ANTIBODIES

(71) Applicants: INSERM (INSTITUT NATIONAL DE LA SANTÉ ET DE LA RECHERCHE MÉDICAL), Paris Cedex 13 (FR); INSTITUT JEAN PAOLI & IRENE CALMETTES, Marseille (FR); CENTRE NATIONAL DE LA RECHERCHE SCIENTIFIQUE, Paris (FR); UNIVERSITÉ D'AIX MARSEILLE, Marseille Cedex 7 (FR); UNIVERSITE DE PARIS, Paris (FR); ALDERAAN BIOTECHNOLOGY, Paris (FR); UNIVERSITE PARIS EST CRETEIL VAL DE MARNE, CRETEIL (FR)

(72) Inventors: Daniel OLIVE, Marseille (FR); Armand BENSUSSAN, Paris (FR); Jérôme GIUSTINIANI, Saint-Maur-des-Fossés (FR); Arnaud FOUSSAT, Valbonne (FR)

(73) Assignees: INSERM (INSTITUT NATIONAL DE LA SANTÉ ET DE LA RECHERCHE MÉDICALE), Paris Cedex 13 (FR); INSTITUT JEAN PAOLI & IRENE CALMETTES, Marseille (FR); CENTRE NATIONAL DE LA RECHERCHE SCIENTIFIQUE, Paris (FR); UNIVERSITÉ D'AIX MARSEILLE, Marseille Cedex (FR); UNIVERSITE DE PARIS, Paris (FR); ALDERAAN BIOTECHNOLOGY, Paris (FR); UNIVERSITE PARIS EST CRETEIL VAL DE MARNE, CRETEIL (FR)

(21) Appl. No.: 17/612,347

(22) PCT Filed: May 20, 2020
(86) PCT No.: PCT/EP2020/064154

§ 371 Date: Nov. 18, 2021

Related U.S. Application Data
(60) Provisional application No. 62/924,579, filed on Oct. 22, 2019.
(60) Provisional application No. 62/965,784, filed on Jan. 24, 2020.

(30) Foreign Application Priority Data
May 20, 2019 (EP) 19305640.5

(51) Int. Cl. C07K 16/28; A61P 35/00; C12N 15/63

(52) U.S. Cl. CPC C07K 16/2896; A61P 35/00; C07K 16/2866; C07K 2317/732; C07K 2317/24; C07K 2319/00; C12N 15/63

(57) ABSTRACT

Novel anti-CD25 antibodies and antigen-bind fragments thereof that do not inhibit the binding of interleukin-2 (IL-2) to CD25, and the use thereof for treating cancer or an infectious disease. Also, fusion proteins including the antibodies and antigen-bind fragments, nucleic acids encoding the antibodies and antigen-bind fragments, and an expression vectors including the nucleic acids. Further, pharmaceutical compositions including the fusion proteins or the antibodies and antigen-bind fragments.

### FIELD OF INVENTION

The present invention relates to the field of treatment of cancer and infectious diseases, and in particular discloses novel anti-CD25 antibodies that may be used for treating cancer and infectious diseases.

### BACKGROUND

Regulatory T cells (Tregs) are key mediators of immune tolerance generally protecting the body against autoimmunity. However, in cancer, Tregs appear to play a controversial role.

Indeed, during cancer, tumor microenvironment often favors differentiation and recruitment of Tregs, which may thus suppress antitumor effector T cell function. Tregs may thus constitute a major obstacle for immunotherapy. This phenomenon has been described in many human cancers and in most mouse models of tumor growth, wherein the frequency of Tregs and their suppressor functions are increased as compared to those reported for healthy subjects. In particular, it has been shown that Tregs accumulate in the tumor in the presence of tumor-derived chemokines, and once in place, proceed to prevent or blunt antitumor responses mediated by immune cells infiltrating the tumor microenvironment. Therefore, Tregs which accumulate can be viewed as one of multiple attempts of the tumor to promote its own escape from the host immune system by silencing antitumor immune effector cells.

In view of their enhanced capability to suppress antitumor functions of effector T cells, Tregs have been perceived as mediators of tumor escape that need to be unequivocally silenced or eliminated if antitumor functions are to be restored.

Tregs were first described by Sakaguchi et al. as a circulating subset of murine CD4⁺ T cells expressing constitutively high levels of CD25, the interleukin-2 receptor a chain that binds to interleukin-2 (IL-2) and regulates development and homeostasis of Tregs.

Different studies have analyzed the interaction between IL-2 and CD25 in murine models. It has been shown that the blockade of the binding of IL-2 to CD25, with an anti-CD25 antibody, such as, for example PC61, in tumor-bearing mice results in a loss of both FoxP3 expression and Tregs suppressive function. These results suggest that the deprivation of IL2 represents a promising method for preventing cancer development. Recently, Vargas et al. have developed an Fc optimized form of the PC61 antibody allowing intra-tumoral Tregs depletion via antibody-dependent cellular cytotoxicity (ADCC) or complement-mediated cytotoxicity mechanisms (CDC) offering a significant therapeutic benefit in murine tumor models. Given the physiological importance of the IL-2 pathway in Tregs, the blockade of said pathway thus seemed to be a powerful and promising antitumoral immunotherapy.

There remains a need for effective antitumoral immunotherapies.

### SUMMARY

The present invention relates to an isolated anti-human CD25 antibody or antigen-binding fragment thereof, wherein said antibody does not inhibit the binding of interleukin-2 (IL-2) to CD25, preferably wherein said antibody is monoclonal.

In one embodiment, the antibody is a chimeric antibody, a humanized antibody or a human antibody.

In one embodiment, the variable region of the heavy chain (HCVR) comprises at least one, preferably at least two, more preferably the three following complementary-determining regions (CDRs):

CDR1:
(SEQ ID NO: 1) [symbol]
HAMA,
wherein [symbol] is D or N:
CDR2:
(SEQ ID NO: 2)
YISYDGDNTYYRDSVKG;
and
CDR3:
(SEQ ID NO: 3)
GGNSGYD;

or any CDR having an amino acid sequence that shares at least about 70% of identity with SEQ ID NO: 1-3; and/or
the variable region of the light chain (LCVR) comprises at least one, preferably at least two, more preferably the three following CDRs:

CDR1:
(SEQ ID NO: 4)
K [symbol] SQNVNKF [symbol] N,
wherein [symbol] is A or G
and
wherein [symbol] is L or V;
CDR2:
(SEQ ID NO: 5)
GTNSLQT;
and
CDR3:
(SEQ ID NO: 6)
QQY [symbol] SWPWT,
wherein [symbol] is S or T;

or any CDR having an amino acid sequence that shares at least about 70% of identity with SEQ ID NO: 4-6.

In one embodiment,
(i) the HCVR comprises at least one, preferably at least two, more preferably the three CDRs as defined hereinabove, and
(ii) the LCVR comprises at least one, preferably at least two, more preferably the three CDRs as defined hereinabove.

In one embodiment,
(i) the HCVR comprises the following CDRs:

CDR1:
(SEQ ID NO: 1) [symbol]
HAMA,
wherein [symbol] is D or N;
CDR2:
(SEQ ID NO: 2)
YISYDGDNTYYRDSVKG;
and
CDR3:
(SEQ ID NO: 3)
GGNSGYD;

and
(ii) the LCVR comprises the following CDRs:

CDR1:
(SEQ ID NO: 4)
K [symbol] SQNVNKF [symbol] N,
wherein [symbol] is A or G
and
wherein [symbol] is L or V;
CDR2:
(SEQ ID NO: 5)
GTNSLQT;
and
CDR3:
(SEQ ID NO: 6)
QQY [symbol] SWPWT,
wherein [symbol] is S or T;

or any CDR having an amino acid sequence that shares at least about 70% of identity with said SEQ ID NO: 1-6.

In one embodiment,
the HCVR comprises the following CDRs:

CDR1:
(SEQ ID NO: 7)
DHAMA;
CDR2:
(SEQ ID NO: 2)
YISYDGDNTYYRDSVKG;
and
CDR3:
(SEQ ID NO: 3)
GGNSGYD;

and
the LCVR of said antibody or antigen-binding fragment thereof comprises the following CDRs:

CDR1:
(SEQ ID NO: 8)
KASQNVNKFLN;
CDR2:
(SEQ ID NO: 5)
GTNSLQT;
and
CDR3:
(SEQ ID NO: 9)
QQYSSWPWT.

In one embodiment,
(i) the HCVR of said antibody or antigen-binding fragment thereof comprises the following CDRs:

CDR1:
(SEQ ID NO: 10)
NHAMA;
CDR2:
(SEQ ID NO: 2)
YISYDGDNTYYRDSVKG;
and
CDR3:
(SEQ ID NO: 3)
GGNSGYD;

and
(ii) the LCVR of said antibody or antigen-binding fragment thereof comprises the following CDRs:

(SEQ ID NO: 11)
CDR1: KASQNVNKFVN;
(SEQ ID NO: 5)
CDR2: GTNSLQT;
and
(SEQ ID NO: 9)
CDR3: QQYSSWPWT.

In one embodiment,
(i) the HCVR of said antibody or antigen-binding fragment thereof comprises the following CDRs:

(SEQ ID NO: 10)
CDR1: NHAMA;
(SEQ ID NO: 2)
CDR2: YISYDGDNTYYRDSVKG;
and
(SEQ ID NO: 3)
CDR3: GGNSGYD;

and
(ii) the LCVR of said antibody or antigen-binding fragment thereof comprises the following CDRs:

(SEQ ID NO: 12)
CDR1: KGSQNVNKFLN;
(SEQ ID NO: 5)
CDR2: GTNSLQT;
and
(SEQ ID NO: 13)
CDR3: QQYTSWPWT.

In one embodiment, the antibody or antigen-binding fragment thereof is a bispecific antibody.

The present invention further relates to a fusion protein comprising the antibody or antigen-binding fragment thereof as described herein.

The present invention further relates to a nucleic acid encoding the isolated antibody or antigen-binding fragment thereof, or the fusion protein, as described herein.

The present invention further relates to an expression vector comprising the nucleic acid as described herein.

In one embodiment, said antibody or antigen-binding fragment mediates antibody dependent cellular cytotoxicity, complement dependent cytotoxicity or antibody-dependent phagocytosis.

The present invention further relates to a pharmaceutical composition comprising the isolated antibody or antigen-binding fragment thereof, or the fusion protein, as described herein, and at least one pharmaceutically acceptable excipient.

The present invention further relates to an isolated antibody or antigen-binding fragment thereof as described herein, or the fusion protein as described herein, for use as a medicament.

The present invention further relates to an isolated antibody or antigen-binding fragment thereof as described herein, for use in treating a cancer or an infectious disease.

The present invention further relates to a fusion protein as described herein, for use in treating a cancer or an infectious disease.

The present invention further relates to a combination of an immunotherapy and an antibody or antigen-binding fragment thereof as described herein, for use in treating a cancer or an infectious disease in a subject.

The present invention further relates to a combination of an immunotherapy and a fusion protein as described herein, for use in treating a cancer or an infectious disease in a subject.

The present invention further relates to a method of inducing specific lysis of CD25 positive cells without inhibiting IL-2 signaling in T-cells, the method comprising the step of administering to a subject a therapeutically effective amount of the isolated antibody or antigen-binding fragment as disclosed herein, or a therapeutically effective amount of the fusion protein as described herein, or a therapeutically effective amount of the pharmaceutical composition as disclosed herein. In some embodiments, the subject is receiving or has received an immunotherapy.

The present invention further relates to a method comprising the step of administering to a subject an immunotherapy, wherein the subject has received or is receiving a therapeutically effective amount of the isolated antibody or antigen-binding fragment as disclosed herein, or a therapeutically effective amount of the fusion protein as described herein, or a therapeutically amount of the pharmaceutical composition as disclosed herein. In some embodiments, the therapeutically effective amount is an amount effective to induce specific lysis of CD25 positive cells without inhibiting IL-2 signaling in T-cells.

### BRIEF DESCRIPTION OF THE DRAWINGS

FIG. 1 is a histogram showing the binding of antibodies of the present invention (ALD25H1, ALD25H2 and ALD25H4) or of a control antibody on a CD25 positive cell line.

FIG. 2 is a histogram showing the binding competition of antibodies of the present invention (ALD25H1, ALD25H2 and ALD25H4) with the mAb 7G7B6 (an IL-2 non-competitive antibody).

FIG. 3 is a histogram showing the impact of antibodies of the present invention (ALD25H1, ALD25H2 and ALD25H4) on IL-2 induced effector T cell proliferation, as compared to an IgG1 control antibody or to basiliximab.

FIG. 4 is a combination of a graph (A) and a histogram (B) showing antibody-dependent cell-mediated cytotoxicity (ADCC) induced by antibodies of the present invention. FIG. 4A shows the lysis of CD25 positive cells induced by the incubation with antibodies of the present invention (ALD25H1, ALD25H2 and ALD25H4) or with basiliximab. FIG. 4B represents the percentage of CFSE+7AAD+cells, which correspond to the SUDHL-1 cells apoptosis, induced by the incubation with antibodies of the present invention (ALD25H2 and ALD25H4) at 1 or 10 μg/mL, as compared with an IgG1 control antibody. Data are represented as means±SEM.

FIG. 5 is a graph showing the impact of the antibodies of the present invention (ALD25H1, ALD25H2 and ALD25H4), or 7G7B6 or the mAb MA-251 (an IL-2 non-competitive antibody) or Basiliximab on IL-2 induced T-cell proliferation. “Isotype Ctl”: isotype control antibody. Error bars represent sem.

FIG. 6 is a combination of five histograms (A to E) showing the impact of the antibody of the present invention (ALD25H4) on Treg cells depletion. Error bars represent sem. FIG. 6A represents the percentage of CD4⁺CD25⁺FoxP3⁺ Treg cells within tumour infiltrating CD4⁺ T cells in humanized mice treated with ALD25H4, 7G7B6 or with the vehicle. FIG. 6B represents the percentage of CD4⁺CD25⁺FoxP3⁺ Treg cell depletion obtained by anti-CD25 monoclonal antibodies. FIG. 6C represents the percentage of CD4⁺CD25⁺CD127⁻FoxP3⁺ Treg cell within CD45⁺ leucocytes population in tumors induced by MDA-MB-231 or HT29 in humanized mice treated or not with ALD25H4. FIG. 6D represents the percentage of CD4⁺CD25⁺ FoxP3⁻ T effector cell (CD4⁺ T effector cells) within CD45⁺ leucocytes population in tumors induced by MDA-MB-231 or HT29 in humanized mice treated or not with ALD25H4. FIG. 6E represents the percentage of CD8⁺CD25⁺ FoxP3⁻ T effector cell (CD8⁺ T effector cells) within CD45⁺ leucocytes population in tumors induced by MDA-MB-231 or HT29 in humanized mice treated or not with ALD25H4.

FIG. 7 is a histogram showing the impact of antibodies of the present invention (ALD25H4) at 1 or 10 μg/mL on IL-2 binding in vitro, as compared to an IgG1 control antibody or to basiliximab.

FIG. 8 is a histogram showing the percentage of antibody dependent phagocytosis (ADCP) induced by an antibody of the present invention (ALD25H4), as compared to an IgG1 control antibody. Data are represented as means±SEM.

FIG. 9 is a combination of three histograms (A, B and C) showing the impact of antibodies of the present invention on Treg cells depletion within the CD45+ lymphocyte population. FIG. 9A represents the percentage of Treg cells within the CD45+ lymphocyte population following incubation with an IgG1 control antibody, antibodies of the present invention (ALD25H1, ALD25H2 and ALD25H4) or basiliximab FIG. 9B represents the percentage of CD4+ T effector cells within the CD45+ lymphocyte population following incubation with an IgG1 control antibody, antibodies of the present invention (ALD25H1, ALD25H2 and ALD25H4) or basiliximab FIG. 9C represents the percentage of CD8+ T effector cells within the CD45+ lymphocyte population following incubation with an IgG1 control antibody, antibodies of the present invention (ALD25H1, ALD25H2 and ALD25H4) or basiliximab Data are represented as means±SEM.

### DEFINITIONS

In the present invention, the following terms have the following meanings:

“About”, preceding a figure encompasses plus or minus 10%, or less, of the value of said figure. It is to be understood that the value to which the term “about” refers is itself also specifically, and preferably, disclosed.

“Adnectins”, also known as monobodies, is well known in the art and refer to proteins designed to bind with high affinity and specificity to antigens. They belong to the class of molecules collectively called “antibody mimetics”.

“Alphabody” that may also be referred to as Cell-Penetrating Alphabodies, refer to a type of antibody mimetics consisting of small 10 kDa proteins engineered to bind to a variety of antigens. Alphabodies are able to reach and bind to intracellular protein targets.

“Affibodies” are well-known in the art and refer to affinity proteins based on a 58 amino acid residue protein domain, derived from one of the IgG binding domain of staphylococcal protein A (Frejd & Kim, 2017. Exp Mol Med. 49(3):e306; U.S. Pat. No. 5,831,012).

“Affilins” are well known in the art and refer to artificial proteins designed to selectively bind antigens. They resemble antibodies in their affinity and specificity to antigens but not in structure which makes them a type of antibody mimetic.

“Affinity” and “avidity” are well-known in the art and are used to defined the strength of an antibody-antigen complex. Affinity measures the strength of interaction between an epitope and an antigen binding site on an antibody. It may be expressed by an affinity constant K_{A} or by a dissociation constant K_{D}. Avidity (or functional affinity) gives a measure of the overall strength of an antibody-antigen complex. It may depend on different parameters, including in particular the affinity of the antibody or antigen-binding fragment thereof for an epitope, (ii) the valency of both the antibody and the antigen and (iii) structural arrangement of the parts that interact.

As used herein, the terms “antibody” and “immunoglobulin” may be used interchangeably and refer to a protein having a combination of two heavy and two light chains whether or not it possesses any relevant specific immunoreactivity. “Antibodies” refers to such assemblies which have significant known specific immunoreactive activity to an antigen of interest (e.g., human CD25). The term “anti-hCD25 antibodies” is used herein to refer to antibodies which exhibit immunological specificity for human CD25 protein. As explained elsewhere herein, “specificity” for human CD25 does not exclude cross-reaction with species homologues of hCD25, such as, for example, with simian CD25.

Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention.

As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the VH and VL chains.

“Affitins” refer to highly stable engineered affinity proteins, originally derived from Sac7d and Sso7d, two 7 kDa DNA-binding polypeptides from Sulfolobus genera.

“Anticalins” are well known in the art and refer to an antibody mimetic technology, wherein the binding specificity is derived from lipocalins. Anticalins may also be formatted as dual targeting protein, called Duocalins.

The term “antigen-binding fragment”, as used herein, refers to a part or region of the antibody according to the present invention, which comprises fewer amino acid residues than the whole antibody. An “antigen-binding fragment” binds antigen and/or competes with the whole antibody from which it was derived for antigen binding (e.g., specific binding to human CD25). Antibody antigen-binding fragments encompasses, without any limitation, single chain antibodies, Fv, Fab, Fab′, Fab′-SH, F(ab)′₂, Fd, defucosylated antibodies, diabodies, triabodies and tetrabodies.

“Armadillo repeat protein-based scaffold”, as used herein, refers to a type of antibody mimetics corresponding to artificial peptide binding scaffolds based on armadillo repeat proteins. Armadillo repeat proteins are characterized by an armadillo domain, composed of tandem armadillo repeats of approximately 42 amino acids, which mediates interactions with peptides or proteins.

“Atrimers” are well known in the art and refers to binding molecules for target protein that trimerize as a perquisite for their biological activity. They are relatively large compared to other antibody mimetic scaffolds.

“Avimers” are well known in the art and refer to an antibody mimetic technology.

As used herein, the term “CD25” refers to any native CD25 from any vertebrate source, including mammals such as primates (e.g., humans) and rodents (e.g., mice and rats), unless otherwise indicated. The Interleukin-2 receptor alpha chain (also called CD25) protein is encoded by the IL2RA gene. Two forms of the IL-2 receptor were described: the first one comprising the alpha subunit (CD25), the beta subunit (CD122) and the gamma subunit (CD132), and the second one comprising only the beta and gamma subunits (i.e., CD122 and CD132). The term encompasses “full-length” or unprocessed CD25 as well as any form of CD25 that results from processing in the cell. The term also encompasses naturally occurring variants of CD25 (e.g., splice variants or allelic variants). In certain embodiments CD25 is human CD25. For example, CD25 is expressed by activated T lymphocytes and activated B lymphocytes responding to antigen or mitogen stimulation. CD25 is also expressed by regulatory T cells (CD25^{high} FoxP3⁺ regulatory T cells). In one embodiment, CD25 refers to human CD25 (Uniprot accession number P01589).

As used herein, the term “CDR” or “complementarity determining region” means the non-contiguous antigen combining sites found within the variable region of both heavy and light chain polypeptides. The precise amino acid sequence boundaries of a given CDR can be determined using any of a number of well-known schemes, including those described by Kabat et al. (1991), “Sequences of Proteins of Immunological Interest” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. More recently, a universal numbering system has been developed and widely adopted, ImMunoGeneTics (IMGT) Information System® (Lefranc et al., Nucleic Acids Res. 27: 209-212 1999). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues may be readily identified. This information can be used in grafting and replacement of CDR residues from immunoglobulins of one species into an acceptor framework from, typically, a human antibody. Correspondence between the Kabat numbering and the IMGT unique numbering system is also well known to one skilled in the art (e.g., Lefranc et al., supra). Thus, in one embodiment, by CDR regions or CDR, it is intended to indicate the hypervariable regions of the heavy and light chains of the immunoglobulins as defined by IMGT® numbering system (e.g. Lefranc et al., supra).

“DARPins” (Designed Ankyrin Repeat Proteins) are well known in the art and refer to an antibody mimetic DRP (designed repeat protein) technology developed to exploit the binding abilities of non-antibody polypeptides.

“Diabodies”, as used herein, refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains. Diabodies are described more fully in Patent EP0404097, Patent application WO1993011161; and Holliger et al., 1993. Proc Natl Acad Sci USA. 90(14):6444-8.

“Domain antibodies” are well-known in the art and refer to the smallest functional binding units of antibodies, corresponding to the variable regions of either the heavy or light chains of antibodies.

“Domain kunitz peptide” refer to a type of antibody mimetics, and is based on the active domains of proteins inhibiting the function of proteases.

The term “effector T cells” refers to a group of cells that includes several T cell types (e.g., CD4⁺ and CD8⁺ T cells). It includes helpers T cells (Th cells) that help other leukocytes in immunologic processes, including maturation of B cells into plasma cells and memory B cells and cytotoxic T cells (Tc cells, CTLs, T-killer cells, killer T cells) that destroy virus-infected cells and tumor cells, and are also implicated in transplant rejection.

As used herein, the term “epitope” refers to a specific arrangement of amino acids located on a protein or proteins to which an antibody or antigen-binding fragment thereof or an antibody mimetic binds. Epitopes often consist of a chemically active surface grouping of molecules such as amino acids or sugar side chains, and have specific three dimensional structural characteristics as well as specific charge characteristics. Epitopes can be linear (or sequential) or conformational, i.e., involving two or more sequences of amino acids in various regions of the antigen that may not necessarily be contiguous.

“Evasins” are well known in the art and refer to a class of chemokine-binding proteins.

As used herein, the term “framework region” or “FR region” includes the amino acid residues that are part of the variable region, but are not part of the CDRs (e.g., using the IMGT® numbering definition of CDRs). The framework regions for the light chain are similarly separated by each of the LCVR's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof.

“Fynomers” are well known in the art and refer to proteins that belong to the class of antibody mimetic. They are attractive binding molecules due to their high thermal stability and reduced immunogenicity.

“Fv”, as used herein, refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

As used herein, the term “heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or antigen-binding fragment thereof according to the present invention may comprise the Fc region of an immunoglobulin heavy chain (e.g., a hinge portion, a C_{H}2 domain, and a C_{H}3 domain) In another embodiment, the antibody or antigen-binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule.

That is, the antibody or antigen-binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

The term “hypervariable loop” is not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.). Bethesda, MD: U.S. Dep. of Health and Human Services) and the limits of the HVs and the CDRs may be different in some V_{H} and V_{L} domains. The CDRs of the V_{L} and V_{H} domains can typically be defined by the Kabat/Chothia definition as already explained hereinabove.

As used herein, the term “identity” or “identical”, when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”) Identity of related amino acid sequences or nucleic acid sequences can be readily calculated by known methods. Such methods include, but are not limited to, those described in Lesk A. M. (1988). Computational molecular biology: Sources and methods for sequence analysis. New York, N.Y.: Oxford University Press; Smith D. W. (1993). Biocomputing: Informatics and genome projects. San Diego, Calif.: Academic Press; Griffin A. M. & Griffin H. G. (1994). Computer analysis of sequence data, Part 1. Totowa, N.J.: Humana Press; von Heijne G. (1987). Sequence analysis in molecular biology: treasure trove or trivial pursuit. San Diego, Calif.: Academic press; Gribskov M. R. & Devereux J. (1991). Sequence analysis primer. New York, N.Y.: Stockton Press; Carillo et al., 1988. SIAM J Appl Math. 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, Wis.; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

The term “interleukin-2” or “IL-2” as used herein, refers to any native IL-2 from any vertebrate source, including mammals such as primates (e.g., humans) and rodents (e.g., mice and rats), unless otherwise indicated. The term encompasses unprocessed IL-2 as well as any form of IL-2 that results from processing in the cell. The term also encompasses naturally occurring variants of IL-2 (e.g., splice variants or allelic variants).

“Knottin” (that may also be referred to as inhibitor cystine not) refer to an antibody mimetic comprising a protein structural motif containing three disulfide bridges.

As used herein, the term “mammal”, refers to any mammal, including humans, domestic and farm animals, and zoo, sports, or pet animals, such as dogs, cats, cattle, horses, sheep, pigs, goats, rabbits, etc. Preferably, the mammal is human.

As used herein, the term “monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or antigen-binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

“Nanobodies” are well-known in the art and refer to antibody-derived therapeutic proteins that contain the unique structural and functional properties of naturally-occurring heavy chain antibodies (Muyldermans, 2013. Annu Rev Biochem. 82:775-97). These heavy chain antibodies may contain a single variable domain (VHH) and two constant domains (C_{H}2 and C_{H}3).

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

As used herein, the term “regulatory T cell” or “Treg cell” refers to a specialized type of T cells, in particular of CD4⁺ T cell, that can suppress the responses of other T cells. Treg cells are generally characterized by expression of CD4, the α-subunit of the IL-2 receptor (CD25), and the transcription factor forkhead box P3 (Foxp3) (Sakaguchi, Annu Rev Immunol 22, 531-62 (2004)) and play a critical role in the induction and maintenance of peripheral self-tolerance to antigens, including those expressed by tumors. More recently, CD8 Tregs have also been described.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding (Plückthun, 1994. Antibodies from Escherichia coli. In Rosenberg & Moore (Eds.), The pharmacology of monoclonal antibodies. Handbook of Experimental Pharmacology, 113:269-315. Springer: Berlin, Heidelberg).

As used herein, the term “subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human

The term “therapeutically effective amount” refers to the level or amount of an antibody as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

As used herein, the term “treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a cancer or an infection if, after receiving a therapeutic amount of an antibody according to the methods of the present invention, the patient shows observable and/or measurable reduction in or absence of one or more of the following: reduction in the number of cancer cells (or tumor size), or pathogenic cells; reduction in the percent of total cells that are cancerous or pathogenic; and/or relief to some extent, one or more of the symptoms associated with the specific disease or condition; reduced morbidity and mortality, and improvement in quality of life issues. The above parameters for assessing successful treatment and improvement in the disease are readily measurable by routine procedures familiar to a physician.

As used herein, the term “tumor infiltrating Tregs” relates to CD25^{+/hi} Foxp3⁺ regulatory T cells that accumulate within neoplastic lesions as a result of several distinct mechanisms, including increased infiltration, local expansion, survival advantage and in situ development from conventional CD4⁺ or CD8⁺ cells.

“Unibodies” are well known in the art and refer to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

As used herein, the term “variable” refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site. The first, second and third hypervariable loops of the Vλ light chain domain are referred to herein as L1 (λ), L2 (λ) and L3 (λ) and may be defined as comprising residues 24-33 (L1 (λ), consisting of 9, 10 or 11 amino acid residues), 49-53 L2 (λ), consisting of 3 residues) and 90-96 (L3 (λ), consisting of 6 residues) in the V_{L} domain (Morea et al., 2000. Methods. 20(3):267-79). The first, second and third hypervariable loops of the Vκ light chain domain are referred to herein as L1 (λ), L2 (λ) and L3 (λ) and may be defined as comprising residues 25-33 (L1 (κ), consisting of 6, 7, 8, 11, 12 or 13 residues), 49-53 (L2 (κ), consisting of 3 residues) and 90-97 (L3 (κ), consisting of 6 residues) in the V_{L} domain (Morea et al., 2000. Methods. 20(3):267-79). The first, second and third hypervariable loops of the V_{H} domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., 2000. Methods. 20(3):267-79). Unless otherwise indicated, the terms L1, L2 and L3 respectively refer to the first, second and third hypervariable loops of a VL domain, and encompass hypervariable loops obtained from both Vκ and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

“Versabodies” are well known in the art and refer to another antibody mimetic technology. They are small proteins of 3-5 kDa with >15% cysteines, which form a high disulfide density scaffold, replacing the hydrophobic core the typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

### DETAILED DESCRIPTION

Despite the apparent promise of IL-2 pathway blockade as an antitumoral immunotherapy, the manipulation of the IL-2 pathway should be carefully examined as it modulates both immuno-stimulatory and immuno-regulatory functions. Indeed, while the IL-2 pathway plays an important role in regulating immune responses and maintaining peripheral self-tolerance, it also acts as a T cell growth factor, essential for the proliferation and survival of T cells as well as for the generation of effector and memory T cells. Furthermore, IL-2 receptors are also transiently expressed in effector T cells and myeloid dendritic cells, and therefore IL-2 pathway manipulation could cause unpredicted outcomes, such as, for example, an alteration of antitumor effector T cells, in particular of CD8⁺ effector T cells, function, resulting in cancer progression.

As a component of the immune system, effector CD8³⁰ T cells have important roles in suppressing tumors. For example, effector CD8³⁰ T cells can kill tumor cells with cytotoxic molecules, such as granzymes and perforin. IFN-γ, which is produced by CD8⁺ T cells, can increase the expression of MHC class I antigens by tumor cells, thereby rendering them better targets for CD8³⁰ T cells. Thus, during cancer, effector CD8³⁰ T cells are critical for the elimination of neoplastic cells.

Here, the Applicants aimed to eliminate or silence Tregs while maintaining an efficient effector T cells response during cancer. Thus, the present invention relates to novel anti-CD25 antibodies that exhibit a potent anti-cancer effect, in particular by depleting Tregs, without blocking of the IL-2 signaling pathway, thereby allowing IL-2 to stimulate effector T cells.

The present invention relates to an isolated protein which binds to human CD25 (hCD25).

In one embodiment, the isolated protein according to the present invention is an isolated antibody or antigen-binding fragment thereof, wherein said antibody or antigen-binding fragment thereof binds to human CD25 (hCD25).

An “isolated protein”, and in particular an “isolated antibody”, as used herein, is intended to refer to a protein, in particular an antibody that is substantially free of other proteins or antibodies having different antigenic specificities (e.g., an isolated protein or antibody that specifically binds hCD25 is substantially free of proteins or antibodies that specifically bind antigens other than hCD25). An isolated protein, in particular an isolated antibody, that specifically binds hCD25 may, however, have cross-reactivity to other antigens, such as hCD25 molecules from other species. Moreover, an isolated protein or antibody may be substantially free of other cellular material and/or chemicals, in particular those that would interfere with diagnostic or therapeutic uses of the protein or antibody, including without limitation, enzymes, hormones, and other proteinaceous or non-proteinaceous components.

In one embodiment, the isolated protein, in particular the isolated antibody or antigen-binding fragment thereof is purified.

In one embodiment, the isolated protein or antibody or antigen-binding fragment thereof is purified to:
(1) greater than 80%, 85%, 90%, 91%, 92%, 93%, 94%, 95% or more by weight of protein or of antibody or antigen-binding fragment thereof as determined by the Lowry method, and most preferably more than 96%, 97%, 98% or 99% by weight;
(2) a degree sufficient to obtain at least 15 residues of N-terminal or internal amino acid sequence by use of a spinning cup sequenator; or
(3) homogeneity as shown by SDS-PAGE under reducing or non-reducing conditions and using Coomassie blue or, preferably, silver staining.

According to the present invention, the isolated protein, in particular the isolated antibody or antigen-binding fragment thereof does not inhibit the signaling of IL-2 via CD25. In one embodiment, the isolated protein does not inhibit the binding of interleukin-2 (IL-2) to human CD25. In one embodiment, the isolated antibody or antigen-binding fragment thereof does not inhibit the binding of interleukin-2 (IL-2) to human CD25, and may thus be referred herein as a “non-blocking antibody”.

In one embodiment, the protein according to the present invention inhibits less than 50% of the IL-2 signaling compared to IL-2 signaling in the absence of the protein. In one embodiment, the protein according to the present invention inhibits less than 45%, 40%, 35%, 30%, 25%, 20%, 15%, 10%, 5% of the IL-2 signaling compared to IL-2 signaling in the absence of the protein.

In one embodiment, the antibody or antigen-binding fragment thereof according to the present invention inhibits less than 50% of the IL-2 signaling compared to IL-2 signaling in the absence of the antibody or antigen-binding fragment thereof. In one embodiment, the antibody or antigen-binding fragment thereof according to the present invention inhibits less than 45%, 40%, 35%, 30%, 25%, 20%, 15%, 10%, 5% of the IL-2 signaling compared to IL-2 signaling in the absence of the antibody or antigen-binding fragment thereof.

Methods for measuring the IL-2 signaling are well known in the art and comprise, for example, the measurement of the induction of IL-2 receptor signaling (e.g., by detection of phosphorylated STAT5a), the induction of proliferation (e.g., by detection of Ki-67 using in particular CellTrace™ Cell Proliferation Kits, by direct assessment of T cell proliferation in the presence of IL-2, in MLR experiments (comprising, for example, the activation of cells with CD3 and CD28 in the presence of IL-2), or using cell lines that depend on IL-2 to proliferate, such as, for example CTLL2 cell line) and/or the up-regulation of expression of activation markers (such as e.g., CD25, CD69, cytotoxic molecules, such as, for example, granzyme B, and the like).

In one embodiment, the protein of the present invention does not inhibit the proliferation and/or activation of CD4⁺ and CD8⁺ T cells. In one embodiment, the protein of the present invention does not inhibit the IL-2 induced proliferation of CD4⁺ and CD8⁺ T cells.

In one embodiment, the antibody or antigen-binding fragment thereof of the present invention does not inhibit the proliferation and/or activation of CD4⁺ and CD8⁺ T cells. In one embodiment, the antibody or antigen-binding fragment thereof of the present invention does not inhibit the IL-2 induced proliferation of CD4⁺ and CD8⁺ T cells (an example of a method that may be used for measuring IL-2 induced proliferation of T cells is provided in the Example part). In one embodiment, the antibody or antigen-binding fragment thereof of the present invention inhibits the IL-2 induced proliferation of CD4⁺ and CD8⁺ T cells by less than 30%, preferably less than 25% or less, as compared to the IL-2 induced proliferation of CD4⁺ and CD8⁺ T cells using an isotype control antibody.

In another embodiment, the protein according to the present invention does not inhibit the phosphorylation of STATSa in CD4⁺ and CD8⁺ T cells.

In another embodiment, the antibody or antigen-binding fragment thereof according to the present invention does not inhibit the phosphorylation of STAT5a in CD4⁺ and CD8⁺ T cells.

In one embodiment, the protein according to the present invention inhibits less than 50% of the IL-2 binding to CD25 as compared to IL-2 binding to CD25 in the absence of the protein. In one embodiment, the protein according to the present invention inhibits less than 45%, 40%, 35%, 30%, 25%, 20%, 15%, 10%, 5% of the IL-2 binding to CD25 as compared to IL-2 binding to CD25 in the absence of the protein.

In one embodiment, the antibody or antigen-binding fragment thereof according to the present invention inhibits less than 50% of the IL-2 binding to CD25 as compared to IL-2 binding to CD25 in the absence of the antibody or antigen-binding fragment thereof. In one embodiment, the antibody or antigen-binding fragment thereof according to the present invention inhibits less than 45%, 40%, 35%, 30%, 25%, 20%, 15%, 10%, 5% of the IL-2 binding to CD25 as compared to IL-2 binding to CD25 in the absence of the antibody or antigen-binding fragment thereof.

Methods for measuring the IL-2 binding to CD25 are well known from the skilled artisan and include, without limitation, detection of a labeled-IL-2 on the CD25, such as, for example, of a biotinylated or radiolabeled IL-2 on CD25.

In one embodiment, the protein according to the present invention is specific for human CD25 (hCD25).

In one embodiment, the antibody or antigen-binding fragment thereof according to the present invention is specific for human CD25 (hCD25).

A protein, antibody or antigen-binding fragment thereof is said to be “specific for”, “immunospecific” or to “specifically bind” an antigen if it reacts at a detectable level with said antigen (e.g., CD25), preferably with an affinity constant (K_{A}) of greater than or equal to about 10⁶ M⁻¹, preferably greater than or equal to about 10⁷ M⁻¹, 10⁸ M⁻¹, 5×10⁸ M⁻¹, 10⁹ M⁻¹, 5×10⁹ M⁻¹ or more. Affinity of a protein, or of an antibody or antigen-binding fragment thereof for its cognate antigen is also commonly expressed as an equilibrium dissociation constant (K_{D}). An antibody or antigen-binding fragment thereof is said to be “immunospecific”, “specific for” or to “specifically bind” an antigen if it reacts at a detectable level with said antigen (e.g., CD25), preferably with a K_{D} of less than or equal to 10⁻⁶ M, preferably less than or equal to 10⁻⁷ M, 5.10⁻⁸ M, 10⁻⁸ M, 5.10⁻⁹ M, 10⁻⁹ M or less.

Affinities of antibodies or antigen-binding fragment thereof can be readily determined using conventional techniques, for example, those described by Scatchard, 1949. Ann NY Acad Sci. 51:660-672. Binding properties of an antibody or antigen-binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

In one embodiment, the protein (in particular the antibody or antigen-binding fragment thereof) according to the present invention presents a K_{D} for binding to human CD25 inferior or equal to about 5.10⁻⁹ M, preferably inferior or equal to about 4.10⁻⁹ M or to about 3.10⁻⁹ M. In one embodiment, the K_{D} of the protein of the invention for binding to human CD25 ranges from about 2, 5.10⁻⁹ M to 3, 5.10⁻⁹ M.

In one embodiment, the antibody or antigen-binding fragment thereof according to the present invention is polyclonal.

In another embodiment, the antibody or antigen-binding fragment thereof according to the present invention is monoclonal.

In one embodiment, the antibody or antigen-binding fragment thereof according to the present invention is a molecule selected from the group comprising or consisting of a whole antibody, a humanized antibody, a single chain antibody, a dimeric single chain antibody, a Fv, a Fab, a Fab′, a Fab′-SH, a F(ab)′₂, a Fd, a defucosylated antibody, a bispecific antibody, a diabody, a triabody and a tetrabody.

Antigen-binding fragments of antibodies can be obtained using standard methods. For instance, Fab or F(ab′)₂ fragments may be produced by protease digestion of the isolated antibodies, according to conventional techniques.

It will also be appreciated that antibodies or antigen-binding fragments thereof according to the present invention can be modified using known methods. For example, to slow clearance in vivo and obtain a more desirable pharmacokinetic profile, the antibody or antigen-binding fragment thereof may be modified with polyethylene glycol (PEG). Methods for coupling and site-specifically conjugating PEG to an antibody or antigen-binding fragment thereof are described in, e.g., Leong et al., 2001. Cytokine. 16(3):106-19; Delgado et al., 1996. Br J Cancer. 73(2):175-82.

In one embodiment, the antibody or antigen-binding fragment thereof according to the present invention is a molecule selected from the group comprising or consisting of a unibody, a domain antibody, and a nanobody.

In one embodiment, the isolated protein according to the present invention is an antibody mimetic selected from the group comprising or consisting of an affibody, an alphabody, an armadillo repeat protein based scaffold, a knottin, a kunitz domain peptide, an affilin, an affitin, an adnectin, an atrimer, an evasin, a DARPin, an anticalin, an avimer, a fynomer, a versabody or a duocalin.

In the following, and unless explicitly mentioned otherwise, CDR numbering and definitions are according to the IMGT® numbering system.

In one embodiment, the antibody or antigen-binding fragment thereof according to the present invention comprises a heavy chain variable region (abbreviated herein as HCVR or V_{H}) which comprises at least one, preferably at least two, more preferably the three following complementary-determining regions (CDRs):

(SEQ ID NO: 1)
VH-CDR1: X₄HAMA,
wherein X₄ is D or N:
(SEQ ID NO: 2)
VH-CDR2: YISYDGDNTYYRDSVKG;
and/or
(SEQ ID NO: 3)
VH-CDR3: GGNSGYD;

In one embodiment, the antibody or antigen-binding fragment thereof according to the present invention comprises a HCVR which comprises the three following CDRs:

(SEQ ID NO: 1)
VH-CDR1: X₄HAMA,
wherein X₄ is D or N:
(SEQ ID NO: 2)
VH-CDR2: YISYDGDNTYYRDSVKG;
and
(SEQ ID NO: 3)
VH-CDR3: GGNSGYD.

In one embodiment, the antibody or antigen-binding fragment thereof according to the present invention comprises a HCVR which comprises the three following CDRs:

(SEQ ID NO: 7)
VH-CDR1: DHAMA;
(SEQ ID NO: 2)
VH-CDR2: YISYDGDNTYYRDSVKG;
and
(SEQ ID NO: 3)
VH-CDR3: GGNSGYD.

In one embodiment, the antibody or antigen-binding fragment thereof according to the present invention comprises a HCVR which comprises the three following CDRs:

(SEQ ID NO: 10)
VH-CDR1: NHAMA;
(SEQ ID NO: 2)
VH-CDR2: YISYDGDNTYYRDSVKG;
and
(SEQ ID NO: 3)
VH-CDR3: GGNSGYD.

In one embodiment, the antibody or antigen-binding fragment thereof according to the present invention comprises a light chain variable region (abbreviated herein as LCVR or V_{L}) which comprises at least one, preferably at least two, more preferably the three following complementary-determining regions (CDRs):

VL-CDR1:
(SEQ ID NO: 4)
KX₁SQNVNKFX₂N,
wherein X₁ is A or G and
wherein X₂ is L or V;
VL-CDR2:
(SEQ ID NO: 5)
GTNSLQT;
and/or
VL-CDR3:
(SEQ ID NO: 6)
QQYX₃SWPWT,
wherein X₃ is S or T.

In one embodiment, the antibody or antigen-binding fragment thereof according to the present invention comprises a LCVR which comprises the three following CDRs:

(SEQ ID NO: 4)
VL-CDR1: KX₁SQNVNKFX₂N,
wherein X₁ is A or G
and
wherein X₂ is L or V;
(SEQ ID NO: 5)
VL-CDR2: GTNSLQT;
and
(SEQ ID NO: 6)
VL-CDR3: QQYX₃SWPWT,
wherein X₃ is S or T.

In one embodiment, the antibody or antigen-binding fragment thereof according to the present invention comprises a LCVR which comprises the three following CDRs:

(SEQ ID NO: 8)
VL-CDR1: KASQNVNKFLN;
(SEQ ID NO: 5)
VL-CDR2: GTNSLQT;
and
(SEQ ID NO: 9)
VL-CDR3: QQYSSWPWT.

In one embodiment, the antibody or antigen-binding fragment thereof according to the present invention comprises a LCVR which comprises the three following CDRs:

(SEQ ID NO: 11)
VL-CDR1: KASQNVNKFVN;
(SEQ ID NO: 5)
VL-CDR2: GTNSLQT;
and
(SEQ ID NO: 9)
VL-CDR3: QQYSSWPWT.

In one embodiment, the antibody or antigen-binding fragment thereof according to the present invention comprises a LCVR which comprises the three following CDRs:

(SEQ ID NO: 12)
VL-CDR1: KGSQNVNKFLN;
(SEQ ID NO: 5)
VL-CDR2: GTNSLQT;
and
(SEQ ID NO: 9)
VL-CDR3: QQYSSWPWT.

In one embodiment, the antibody or antigen-binding fragment thereof according to the present invention comprises a LCVR which comprises the three following CDRs:

(SEQ ID NO: 34)
VL-CDR1: KGSQNVNKFVN;
(SEQ ID NO: 5)
VL-CDR2: GTNSLQT;
and
(SEQ ID NO: 9)
VL-CDR3: QQYSSWPWT.

In one embodiment, the antibody or antigen-binding fragment thereof according to the present invention comprises a LCVR which comprises the three following CDRs:

(SEQ ID NO: 8)
VL-CDR1: KASQNVNKFLN;
(SEQ ID NO: 5)
VL-CDR2: GTNSLQT;
and
(SEQ ID NO: 13)
VL-CDR3: QQYTSWPWT.

In one embodiment, the antibody or antigen-binding fragment thereof according to the present invention comprises a LCVR which comprises the three following CDRs:

(SEQ ID NO: 11)
VL-CDR1: KASQNVNKFVN;
(SEQ ID NO: 5)
VL-CDR2: GTNSLQT;
and
(SEQ ID NO: 13)
VL-CDR3: QQYTSWPWT.

In one embodiment, the antibody or antigen-binding fragment thereof according to the present invention comprises a LCVR which comprises the three following CDRs:

(SEQ ID NO: 12)
VL-CDR1: KGSQNVNKFLN;
(SEQ ID NO: 5)
VL-CDR2: GTNSLQT;
and
(SEQ ID NO: 13)
VL-CDR3: QQYTSWPWT.

In one embodiment, the antibody or antigen-binding fragment thereof according to the present invention comprises a LCVR which comprises the three following CDRs:

(SEQ ID NO: 34)
VL-CDR1: KGSQNVNKFVN;
(SEQ ID NO: 5)
VL-CDR2: GTNSLQT;
and
(SEQ ID NO: 13)
VL-CDR3: QQYTSWPWT.

In one embodiment, the antibody or antigen-binding fragment thereof according to the present invention comprises:
a HCVR which comprises at least one, preferably at least two, more preferably the three following CDRs:

(SEQ ID NO: 1)
VH-CDR1: X₄HAMA,
wherein X₄ is D or N:
(SEQ ID NO: 2)
VH-CDR2: YISYDGDNTYYRDSVKG;
and/or
(SEQ ID NO: 3)
VH-CDR3: GGNSGYD;

and
a LCVR which comprises at least one, preferably at least two, more preferably the three following CDRs:

(SEQ ID NO: 4)
VL-CDR1: KX₁SQNVNKFX₂N,
wherein X₁ is A or G and wherein X₂ is L or V;
(SEQ ID NO: 5)
VL-CDR2: GTNSLQT;
and/or
(SEQ ID NO: 6)
VL-CDR3: QQYX₃SWPWT,
wherein X₃ is S or T;

In one embodiment, the antibody or antigen-binding fragment thereof according to the present invention comprises:
a HCVR which comprises the three following CDRs:

(SEQ ID NO: 1)
VH-CDR1: X₄HAMA,
wherein X₄ is D or N:
(SEQ ID NO: 2)
VH-CDR2: YISYDGDNTYYRDSVKG;
and
(SEQ ID NO: 3)
VH-CDR3: GGNSGYD;

and
a LCVR which comprises the three following CDRs:

VL-CDR1:
(SEQ ID NO: 4)
KX₁SQNVNKFX₂N,
wherein X₁ is A or G
and
wherein X₂ is L or V;
VL-CDR2:
(SEQ ID NO: 5)
GTNSLQT;
and
VL-CDR3:
(SEQ ID NO: 6)
QQYX₃SWPWT,
wherein X₃ is S or T;

In one embodiment, any of CDR1, CDR2 and/or CDR3 of the HCVR with SEQ ID NOs 1-3, 7 or 10 can be characterized as having 1, 2, 3 or more amino acids being substituted by a different amino acid.

In one embodiment, any of CDR1, CDR2 and/or CDR3 of the LCVR with SEQ ID NOs 4-6, 8-9, 11-13 or 34 can be characterized as having 1, 2, 3, 4, 5 or more amino acids being substituted by a different amino acid.

In one embodiment, any of CDR1, CDR2 and/or CDR3 of the HCVR with SEQ ID NOs 1-3, 7 or 10 and/or of the LCVR with SEQ ID NOs 4-6, 8-9, 11-13 or 34 can be characterized as having 1, 2, 3, 4, 5 or more amino acids being substituted by a different amino acid.

In one embodiment, any of CDR1, CDR2 and/or CDR3 of the HCVR with SEQ ID NOs 1-3, 7 or 10 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, any of CDR1, CDR2 and/or CDR3 of the LCVR with SEQ ID NOs 4-6, 8-9, 11-13 or 34 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, any of CDR1, CDR2 and/or CDR3 of the HCVR with SEQ ID NOs 1-3, 7 or 10 and/or of the LCVR with SEQ ID NOs 4-6, 8-9, 11-13 or 34 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

In one embodiment, the antibody or antigen-binding fragment thereof according to the present invention comprises:
a HCVR which comprises the three following CDRs:

(SEQ ID NO: 7)
VH-CDR1: DHAMA;
(SEQ ID NO: 2)
VH-CDR2: YISYDGDNTYYRDSVKG;
and
(SEQ ID NO: 3)
VH-CDR3: GGNSGYD;

and
the LCVR of said antibody or antigen-binding fragment thereof comprises the following CDRs:

(SEQ ID NO: 8)
VL-CDR1: KASQNVNKFLN;
(SEQ ID NO: 5)
VL-CDR2: GTNSLQT;
and
(SEQ ID NO: 9)
VL-CDR3: QQYSSWPWT.

In one embodiment, any of CDR1, CDR2 and/or CDR3 of the HCVR with SEQ ID NOs 7, 2-3 and/or of the LCVR with SEQ ID NOs 5, 8-9 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

One example of an antibody comprising a heavy chain comprising CDR1, CDR2 and CDR3 with SEQ ID NOs 7, 2 and 3 and a light chain comprising CDR1, CDR2 and CDR3 with SEQ ID NOs 8, 5 and 9 is Ald25H1.

In one embodiment, the antibody or antigen-binding fragment thereof according to the present invention comprises:
a HCVR which comprises the three following CDRs:

(SEQ ID NO: 10)
VH-CDR1: NHAMA;
(SEQ ID NO: 2)
VH-CDR2: YISYDGDNTYYRDSVKG;
and
(SEQ ID NO: 3)
VH-CDR3: GGNSGYD;

and
the LCVR of said antibody or antigen-binding fragment thereof comprises the following CDRs:

(SEQ ID NO: 11)
VL-CDR1: KASQNVNKFVN;
(SEQ ID NO: 5)
VL-CDR2: GTNSLQT;
and
(SEQ ID NO: 9)
VL-CDR3: QQYSSWPWT.

In one embodiment, any of CDR1, CDR2 and/or CDR3 of the HCVR with SEQ ID NOs 10, 2-3 and/or of the LCVR with SEQ ID NOs 5, 9 and 11 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

One example of an antibody comprising a heavy chain comprising CDR1, CDR2 and CDR3 with SEQ ID NOs 10, 2 and 3 and a light chain comprising CDR1, CDR2 and CDR3 with SEQ ID NOs 11, 5 and 9 is Ald25H2.

In one embodiment, the antibody or antigen-binding fragment thereof according to the present invention comprises:
a HCVR which comprises the three following CDRs:

(SEQ ID NO: 10)
CDR1: NHAMA;
(SEQ ID NO: 2)
CDR2: YISYDGDNTYYRDSVKG;
and
(SEQ ID NO: 3)
CDR3: GGNSGYD;

and
the LCVR of said antibody or antigen-binding fragment thereof comprises the following CDRs:

(SEQ ID NO: 12)
CDR1: KGSQNVNKFLN;
(SEQ ID NO: 5)
CDR2: GTNSLQT;
and
(SEQ ID NO: 13)
CDR3: QQYTSWPWT.

In one embodiment, any of CDR1, CDR2 and/or CDR3 of the HCVR with SEQ ID NOs 10, 2-3 and/or of the LCVR with SEQ ID NOs 5, 12-13 can be characterized as having an amino acid sequence that shares at least 70%, 75%, 80%, 85%, 90%, 95%, 96%, 97%, 98%, 99% or more of identity with the particular CDR or sets of CDRs listed in the corresponding SEQ ID NOs.

One example of an antibody comprising a heavy chain comprising CDR1, CDR2 and CDR3 with SEQ ID NOs 10, 2 and 3 and a light chain comprising CDR1, CDR2 and CDR3 with SEQ ID NOs 12, 5 and 13 is Ald25H4.

In one embodiment, the antibody or antigen-binding fragment thereof according to the present invention comprises a HCVR comprising or consisting of the sequence SEQ ID NO: 35, wherein X₁ is E or Q, X₂ is L or V, X₃ is M or L, X₄ is K or R, X₅ is A or V, X₆ is T or P, X₇ is N or D, X₈ is K or G, X₉ is E or Q, X₁₀ is A or S, X₁₁ is K, R or Q, X₁₂ is S or N, X₁₃ is Y or F, X₁₄ is I or M, X₁₅ is D or N, X₁₆ is S or A, X₁₇ is T or V, X₁₈ is V or T and X₁₉ is M or L.

SEQ ID NO: 35
X₁VQLVESGGGX₂VQPGRSX₃X₄LSCAX₅SGFX₆FSX₇HAMAWVRQAPX₈
KGLX₉WVAYISYDGDNTYYRDSVKGRFTISRDNX₁₀X₁₁X₁₂TLX₁₃LQ
X₁₄X₁₅SLRX₁₆EDTAX₁₇YYCTTGGNSGYDWGQGX₁₈X₁₉VTVSS

In one embodiment, the antibody or antigen-binding fragment thereof according to the present invention comprises a HCVR comprising or consisting of the sequence SEQ ID NO: 20, wherein X₁ is A or V, X₂ is P or T, X₃ is D or N, X₄ is Q or E, X₅ is R, K or Q, X₆ is F or Y and X₇ is M or I.

SEQ ID NO: 20
EVQLVESGGGLVQPGRSMKLSCAX₁SGFX₂FSX₃HAMAWVRQAPKKGLX₄
WVAYISYDGDNTYYRDSVKGRFTISRDNAX₅STLX₆LQX₇DSLRSEDTAT
YYCTTGGNSGYDWGQGVMVTVSS

In one embodiment, the antibody or antigen-binding fragment thereof according to the present invention comprises a LCVR comprising or consisting of the sequence SEQ ID NO: 36, wherein X₁ is F or S, X₂ is N or T, X₃ is A or G, X₄ is V or L, X₅ is L or P, X₆ is E or K, X₇ is R or K, X₈ is R or L, X₉ is I or V, X₁₀ is Y or F, X₁₁ is V or F, X₁₂ is F or Y, X₁₃ is S or T, X₁₄ is G or Q and X₁₅ is L or I.

SEQ ID NO: 36
DIQMTQSPSX₁LSASVGDRVTIX₂CKX₃SQNVNKFX₄NWYQQKX₅GX₆AP
X₇X₈LIYGTNSLQTGX₉PSRFSGSGSGTDX₁₀TLTISSLQPEDX₁₁ATY
X₁₂CQQYX₁₃SWPWTFGX₁₄GTKLEX₁₅K

In one embodiment, the antibody or antigen-binding fragment thereof according to the present invention comprises a LCVR comprising or consisting of the sequence SEQ ID NO: 21, wherein X₁ is A or G, X₂ is L or V and X₃ is S or T.

SEQ ID NO: 21
DIQMTQSPSFLSASVGDRVTINCKX₁SQNVNKFX₂NWYQQKLGEAPRRLI
YGTNSLQTGIPSRFSGSGSGTDYTLTISSLQPEDVATYFCQQYX₃SWPWT
FGGGTKLELK

In one embodiment, the antibody or antigen-binding fragment thereof according to the present invention comprises:
a HCVR comprising or consisting of the sequence SEQ ID NO: 35, wherein X₁ is E or Q, X₂ is L or V, X₃ is M or L, X₄ is K or R, X₅ is A or V, X₆ is T or P, X₇ is N or D, X₈ is K or G, X₉ is E or Q, X₁₀ is A or S, X₁₁ is K, R or Q, X₁₂ is S or N, X₁₃ is Y or F, X₁₄ is I or M, X₁₅ is D or N, X₁₆ is S or A, X₁₇ is T or V, X₁₈ is V or T and X₁₉ is M or L; and
a LCVR comprising or consisting of the sequence SEQ ID NO: 36, wherein X₁ is F or S, X₂ is N or T, X₃ is A or G, X₄ is V or L, X₅ is L or P, X₆ is E or K, X₇ is R or K, X₈ is R or L, X₉ is I or V, X₁₀ is Y or F, X₁₁ is V or F, X₁₂ is F or Y, X₁₃ is S or T, X₁₄ is G or Q and X₁₅ is L or I.

In one embodiment, the antibody or antigen-binding fragment thereof according to the present invention comprises:
a HCVR comprising or consisting of the sequence SEQ ID NO: 20, wherein X₁ is A or V, X₂ is P or T, X₃ is D or N, X₄ is Q or E, X₅ is R, K or Q, X₆ is F or Y and X₇ is M or I; and
a LCVR comprising or consisting of the sequence SEQ ID NO: 21, wherein X₁ is A or G, X₂ is L or V and X₃ is S or T.

In one embodiment, the HCVR with SEQ ID NO: 35 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the HCVR with SEQ ID NO: 20 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the LCVR with SEQ ID NO: 36 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids being substituted by a different amino acid.

In one embodiment, the LCVR with SEQ ID NO: 21 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids being substituted by a different amino acid.

In one embodiment, the HCVR with SEQ ID NO: 35 and/or the LCVR with SEQ ID NO: 36 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the HCVR with SEQ ID NO: 20 and/or the LCVR with SEQ ID NO: 21 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the HCVR with SEQ ID NO: 35 can be characterized as having an amino acid sequence that shares at least 70%, 75%, 80%, 85%, 90%, 95%, 96%, 97%, 98%, 99% or more of identity with SEQ ID NO: 35.

In one embodiment, the HCVR with SEQ ID NO: 20 can be characterized as having an amino acid sequence that shares at least 70%, 75%, 80%, 85%, 90%, 95%, 96%, 97%, 98%, 99% or more of identity with SEQ ID NO: 20.

In one embodiment, the LCVR with SEQ ID NO: 36 can be characterized as having an amino acid sequence that shares at least 70%, 75%, 80%, 85%, 90%, 95%, 96%, 97%, 98%, 99% or more of identity with SEQ ID NO: 36.

In one embodiment, the LCVR with SEQ ID NO: 21 can be characterized as having an amino acid sequence that shares at least 70%, 75%, 80%, 85%, 90%, 95%, 96%, 97%, 98%, 99% or more of identity with SEQ ID NO: 21.

In one embodiment, the HCVR with SEQ ID NO: 35 and/or the LCVR with SEQ ID NO: 36 can be characterized as having an amino acid sequence that shares at least 70%, 75%, 80%, 85%, 90%, 95%, 96%, 97%, 98%, 99% or more of identity with SEQ ID NO: 35 and/or SEQ ID NO: 36, respectively.

In one embodiment, the HCVR with SEQ ID NO: 20 and/or the LCVR with SEQ ID NO: 21 can be characterized as having an amino acid sequence that shares at least 70%, 75%, 80%, 85%, 90%, 95%, 96%, 97%, 98%, 99% or more of identity with SEQ ID NO: 20 and/or SEQ ID NO: 21, respectively.

In one embodiment, the antibody or antigen-binding fragment thereof according to the present invention comprises a HCVR and a LCVR comprising amino acid sequences that are homologous to the amino acid sequences of SEQ ID NO: 35 and SEQ ID NO: 36, respectively, and wherein said antibody or antigen-binding fragment thereof retains the desired functional properties.

In one embodiment, the antibody or antigen-binding fragment thereof according to the present invention comprises a HCVR and a LCVR comprising amino acid sequences that are homologous to the amino acid sequences of SEQ ID NO: 20 and SEQ ID NO: 21, respectively, and wherein said antibody or antigen-binding fragment thereof retains the desired functional properties.

As used herein, the phrase “characterized as having [ . . . ] amino acids being substituted by a different amino acid” in reference to a given sequence, refers to the occurrence, in said sequence, of conservative amino acid modifications.

As used herein, the expression “conservative amino acid modifications” refers to modifications that do not significantly affect or alter the binding characteristics of the antibody or antigen-binding fragment thereof containing the amino acid sequence. Such conservative modifications include amino acid substitutions, additions and deletions. Modifications can be introduced into an antibody or antigen-binding fragment thereof by standard techniques known in the art, such as site-directed mutagenesis and PCR-mediated mutagenesis.

Conservative amino acid substitutions are typically those in which an amino acid residue is replaced with an amino acid residue having a side chain with similar physicochemical properties. Specified variable region and CDR sequences may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acid insertions, deletions and/or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine) and aromatic side chains (e.g., tyrosine, phenylalanine, tryptophan, histidine). Thus, one or more amino acid residues within the CDRs and/or variable regions of the antibody or antigen-binding fragment thereof according to the present invention can be replaced with other amino acid residues from the same side chain family and the altered antibody can be tested for retained function (i.e., the properties set forth herein, such as, e.g., the binding to hCD25) using the assays described herein. In another embodiments, a string of amino acids within the CDRs and/or variable regions of the antibody or antigen-binding fragment thereof according to the present invention can be replaced with a structurally similar string that differs in order and/or composition of side chain family members.

In one embodiment, the antibody or antigen-binding fragment thereof according to the present invention comprises a HCVR comprising or consisting of the sequence SEQ ID NO: 14, SEQ ID NO: 15 or SEQ ID NO: 16. In another embodiment, the antibody or antigen-binding fragment thereof according to the present invention comprises a HCVR comprising or consisting of the sequence SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47.

SEQ ID NO: 14
EVQLVESGGGLVQPGRSMKLSCAASGFPFSDHAMAWVRQAPKKGLQWVAYI
SYDGDNTYYRDSVKGRFTISRDNARSTLFLQMDSLRSEDTATYYCTTGGNS
GYDWGQGVMVTVSS
SEQ ID NO: 15
EVQLVESGGGLVQPGRSMKLSCAASGFTFSNHAMAWVRQAPKKGLEWVAYI
SYDGDNTYYRDSVKGRFTISRDNAKSTLYLQIDSLRSEDTATYYCTTGGNS
GYDWGQGVMVTVSS
SEQ ID NO: 16
EVQLVESGGGLVQPGRSMKLSCAVSGFTFSNHAMAWVRQAPKKGLEWVAYI
SYDGDNTYYRDSVKGRFTISRDNAQSTLYLQMDSLRSEDTATYYCTTGGNS
GYDWGQGVMVTVSS
SEQ ID NO: 37
QVQLVESGGGVVQPGRSLRLSCAASGFTFSNHAMAWVRQAPKKGLEWVAYI
SYDGDNTYYRDSVKGRFTISRDNAQSTLYLQMNSLRAEDTAVYYCTTGGNS
GYDWGQGTLVTVSS
SEQ ID NO: 38
QVQLVESGGGVVQPGRSLRLSCAASGFTFSDHAMAWVRQAPKKGLEWVAYI
SYDGDNTYYRDSVKGRFTISRDNAQSTLYLQMNSLRAEDTAVYYCTTGGNS
GYDWGQGTLVTVSS
SEQ ID NO: 39
QVQLVESGGGVVQPGRSLRLSCAASGFTFSNHAMAWVRQAPGKGLEWVAYI
SYDGDNTYYRDSVKGRFTISRDNAKSTLYLQMNSLRAEDTAVYYCTTGGNS
GYDWGQGTLVTVSS
SEQ ID NO: 40
QVQLVESGGGVVQPGRSLRLSCAASGFTFSDHAMAWVRQAPGKGLEWVAYI
SYDGDNTYYRDSVKGRFTISRDNAKSTLYLQMNSLRAEDTAVYYCTTGGNS
GYDWGQGTLVTVSS
SEQ ID NO: 41
EVQLVESGGGLVQPGRSMKLSCAASGFPFSNHAMAWVRQAPKKGLQWVAYI
SYDGDNTYYRDSVKGRFTISRDNARSTLFLQMDSLRSEDTATYYCTTGGNS
GYDWGQGVMVTVSS
SEQ ID NO: 42
EVQLVESGGGLVQPGRSMKLSCAASGFTFSDHAMAWVRQAPKKGLEWVAYI
SYDGDNTYYRDSVKGRFTISRDNAKSTLYLQIDSLRSEDTATYYCTTGGNS
GYDWGQGVMVTVSS
SEQ ID NO: 43
EVQLVESGGGLVQPGRSMKLSCAVSGFTFSDHAMAWVRQAPKKGLEWVAYI
SYDGDNTYYRDSVKGRFTISRDNAQSTLYLQMDSLRSEDTATYYCTTGGNS
GYDWGQGVMVTVSS
SEQ ID NO: 44
QVQLVESGGGVVQPGRSLRLSCAASGFTFSDHAMAWVRQAPGKGLEWVAYI
SYDGDNTYYRDSVKGRFTISRDNSKSTLYLQMNSLRAEDTAVYYCTTGGNS
GYDWGQGTLVTVSS
SEQ ID NO: 45
QVQLVESGGGVVQPGRSLRLSCAASGFTFSNHAMAWVRQAPGKGLEWVAYI
SYDGDNTYYRDSVKGRFTISRDNSKSTLYLQMNSLRAEDTAVYYCTTGGNS
GYDWGQGTLVTVSS
SEQ ID NO: 46
QVQLVESGGGVVQPGRSLRLSCAASGFTFSDHAMAWVRQAPGKGLEWVAYI
SYDGDNTYYRDSVKGRFTISRDNSKNTLYLQMNSLRAEDTAVYYCTTGGNS
GYDWGQGTLVTVSS
SEQ ID NO: 47
QVQLVESGGGVVQPGRSLRLSCAASGFTFSNHAMAWVRQAPGKGLEWVAYI
SYDGDNTYYRDSVKGRFTISRDNSKNTLYLQMNSLRAEDTAVYYCTTGGNS
GYDWGQGTLVTVSS

In one embodiment, the antibody or antigen-binding fragment thereof according to the present invention comprises a LCVR comprising or consisting of the sequence SEQ ID NO: 17, SEQ ID NO: 18 or SEQ ID NO: 19. In another embodiment, the antibody or antigen-binding fragment thereof according to the present invention comprises a LCVR comprising or consisting of the sequence SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

SEQ ID NO: 17
DIQMTQSPSFLSASVGDRVTINCKASQNVNKFLNWYQQKLGEAPRRLIYG
TNSLQTGIPSRFSGSGSGTDYTLTISSLQPEDVATYFCQQYSSWPWTFGG
GTKLELK
SEQ ID NO: 18
DIQMTQSPSFLSASVGDRVTINCKASQNVNKFVNWYQQKLGEAPRRLIYG
TNSLQTGIPSRFSGSGSGTDYTLTISSLQPEDVATYFCQQYSSWPWTFGG
GTKLELK
SEQ ID NO: 19
DIQMTQSPSFLSASVGDRVTINCKGSQNVNKFLNWYQQKLGEAPRRLIYG
TNSLQTGIPSRFSGSGSGTDYTLTISSLQPEDVATYFCQQYTSWPWTFGG
GTKLELK
SEQ ID NO: 48
DIQMTQSPSSLSASVGDRVTITCKGSQNVNKFLNWYQQKLGEAPRRLIYG
TNSLQTGVPSRFSGSGSGTDYTLTISSLQPEDFATYYCQQYTSWPWTFGQ
GTKLEIK
SEQ ID NO: 49
DIQMTQSPSSLSASVGDRVTITCKASQNVNKFLNWYQQKLGEAPRRLIYG
TNSLQTGVPSRFSGSGSGTDYTLTISSLQPEDFATYYCQQYSSWPWTFGQ
GTKLEIK
SEQ ID NO: 50
DIQMTQSPSSLSASVGDRVTITCKASQNVNKFVNWYQQKLGEAPRRLIYG
TNSLQTGVPSRFSGSGSGTDYTLTISSLQPEDFATYYCQQYSSWPWTFGQ
GTKLEIK
SEQ ID NO: 51
DIQMTQSPSSLSASVGDRVTITCKASQNVNKFLNWYQQKPGKAPRRLIYG
TNSLQTGVPSRFSGSGSGTDYTLTISSLQPEDFATYYCQQYSSWPWTFGQ
GTKLEIK
SEQ ID NO: 52
DIQMTQSPSSLSASVGDRVTITCKASQNVNKFVNWYQQKPGKAPRRLIYG
TNSLQTGVPSRFSGSGSGTDYTLTISSLQPEDFATYYCQQYSSWPWTFGQ
GTKLEIK
SEQ ID NO: 53
DIQMTQSPSSLSASVGDRVTITCKGSQNVNKFLNWYQQKPGKAPRRLIYG
TNSLQTGVPSRFSGSGSGTDYTLTISSLQPEDFATYYCQQYTSWPWTFGQ
GTKLEIK
SEQ ID NO: 54
DIQMTQSPSSLSASVGDRVTITCKASQNVNKFLNWYQQKPGKAPRRLIYG
TNSLQTGVPSRFSGSGSGTDFTLTISSLQPEDFATYYCQQYSSWPWTFGQ
GTKLEIK
SEQ ID NO: 55
DIQMTQSPSSLSASVGDRVTITCKASQNVNKFVNWYQQKPGKAPRRLIYG
TNSLQTGVPSRFSGSGSGTDFTLTISSLQPEDFATYYCQQYSSWPWTFGQ
GTKLEIK
SEQ ID NO: 56
DIQMTQSPSSLSASVGDRVTITCKGSQNVNKFLNWYQQKPGKAPRRLIYG
TNSLQTGVPSRFSGSGSGTDFTLTISSLQPEDFATYYCQQYTSWPWTFGQ
GTKLEIK
SEQ ID NO: 57
DIQMTQSPSSLSASVGDRVTITCKASQNVNKFLNWYQQKPGKAPKLLIYG
TNSLQTGVPSRFSGSGSGTDFTLTISSLQPEDFATYYCQQYSSWPWTFGQ
GTKLEIK
SEQ ID NO: 58
DIQMTQSPSSLSASVGDRVTITCKASQNVNKFVNWYQQKPGKAPKLLIYG
TNSLQTGVPSRFSGSGSGTDFTLTISSLQPEDFATYYCQQYSSWPWTFGQ
GTKLEIK
SEQ ID NO: 59
DIQMTQSPSSLSASVGDRVTITCKGSQNVNKFLNWYQQKPGKAPKLLIYG
TNSLQTGVPSRFSGSGSGTDFTLTISSLQPEDFATYYCQQYTSWPWTFGQ
GTKLEIK

In one embodiment, the antibody or antigen-binding fragment thereof according to the present invention comprises:
a HCVR comprising or consisting of the sequence SEQ ID NO: 14, SEQ ID NO: 15 or SEQ ID NO: 16; and
a LCVR comprising or consisting of the sequence SEQ ID NO: 17, SEQ ID NO: 18 or SEQ ID NO: 19.

In one embodiment, the antibody or antigen-binding fragment thereof according to the present invention comprises:
a HCVR comprising or consisting of the sequence SEQ ID NO: 14, SEQ ID NO: 15, SEQ ID NO: 16, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47; and
a LCVR comprising or consisting of the sequence SEQ ID NO: 17, SEQ ID NO: 18, SEQ ID NO: 19, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody is the Ald25H1 antibody, and the antibody or antigen-binding fragment thereof comprises:
a HCVR comprising or consisting of the sequence SEQ ID NO: 14; and
a LCVR comprising or consisting of the sequence SEQ ID NO: 17.

In one embodiment, the antibody or antigen-binding fragment thereof comprises:
a HCVR comprising or consisting of the sequence SEQ ID NO: 14; and
a LCVR comprising or consisting of the sequence SEQ ID NO: 18.

In one embodiment, the antibody or antigen-binding fragment thereof comprises:
a HCVR comprising or consisting of the sequence SEQ ID NO: 14; and
a LCVR comprising or consisting of the sequence SEQ ID NO: 19.

In one embodiment, the antibody or antigen-binding fragment thereof comprises:
a HCVR comprising or consisting of the sequence SEQ ID NO: 14; and
a LCVR comprising or consisting of the sequence SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 15; and
a LCVR comprising or consisting of the sequence SEQ ID NO: 17.

In one embodiment, the antibody is the Ald25H2 antibody, and the antibody or antigen-binding fragment thereof comprises:
a HCVR comprising or consisting of the sequence SEQ ID NO: 15; and
a LCVR comprising or consisting of the sequence SEQ ID NO: 18.

In one embodiment, the antibody or antigen-binding fragment thereof comprises:
a HCVR comprising or consisting of the sequence SEQ ID NO: 15; and
a LCVR comprising or consisting of the sequence SEQ ID NO: 19.

In one embodiment, the antibody or antigen-binding fragment thereof comprises:
a HCVR comprising or consisting of the sequence SEQ ID NO: 15; and
a LCVR comprising or consisting of the sequence SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 16; and
a LCVR comprising or consisting of the sequence SEQ ID NO: 17.

In one embodiment, the antibody or antigen-binding fragment thereof comprises:
a HCVR comprising or consisting of the sequence SEQ ID NO: 16; and
a LCVR comprising or consisting of the sequence SEQ ID NO: 18.

In one embodiment, the antibody is the Ald25H4 antibody, and the antibody or antigen-binding fragment thereof comprises:
a HCVR comprising or consisting of the sequence SEQ ID NO: 16; and
a LCVR comprising or consisting of the sequence SEQ ID NO: 19.

In one embodiment, the antibody or antigen-binding fragment thereof comprises:
a HCVR comprising or consisting of the sequence SEQ ID NO: 16; and
a LCVR comprising or consisting of the sequence SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 37; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 38; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 39; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 40; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 41; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 42; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 43; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 44; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 45; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 46; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the antibody or antigen-binding fragment thereof comprises:
a HCVR comprising or consisting of the sequence SEQ ID NO: 47; and
a LCVR comprising or consisting of the sequence SEQ ID NO: 17, SEQ ID NO: 18, SEQ ID NO: 19, SEQ ID NO: 48, SEQ ID NO: 49, SEQ ID 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the HCVR with SEQ ID NO: 14, SEQ ID NO: 15, SEQ ID NO: 16, SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the LCVR with SEQ ID NO: 17, SEQ ID NO: 18, SEQ ID NO: 19, SEQ ID NO: 21,SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids being substituted by a different amino acid.

In one embodiment, the HCVR with SEQ ID NO: 14, SEQ ID NO: 15, SEQ ID NO: 16 or SEQ ID NO: 20 and/or the LCVR with SEQ ID NO: 17, SEQ ID NO: 18, SEQ ID NO: 19 or SEQ ID NO: 21 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the HCVR with SEQ ID NO: 14, SEQ ID NO: 15, SEQ ID NO: 16, SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47 and/or the LCVR with SEQ ID NO: 17, SEQ ID NO: 18, SEQ ID NO: 19, SEQ ID NO: 21, SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the HCVR with SEQ ID NO: 14, SEQ ID NO: 15, SEQ ID NO: 16, SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47 can be characterized as having an amino acid sequence that shares at least 70%, 75%, 80%, 85%, 90%, 95%, 96%, 97%, 98%, 99% or more of identity with SEQ ID NO: 14, SEQ ID NO: 15, SEQ ID NO: 16, SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47.

In one embodiment, the LCVR with SEQ ID NO: 17, SEQ ID NO: 18, SEQ ID NO: 19, SEQ ID NO: 21, SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59 can be characterized as having an amino acid sequence that shares at least 70%, 75%, 80%, 85%, 90%, 95%, 96%, 97%, 98%, 99% or more of identity with SEQ ID NO: 17, SEQ ID NO: 18, SEQ ID NO: 19, SEQ ID NO: 21, SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59.

In one embodiment, the HCVR with SEQ ID NO: 14, SEQ ID NO: 15, SEQ ID NO: 16 or SEQ ID NO: 20 and/or the LCVR with SEQ ID NO: 17, SEQ ID NO: 18, SEQ ID NO: 19 or SEQ ID NO: 21 can be characterized as having an amino acid sequence that shares at least 70%, 75%, 80%, 85%, 90%, 95%, 96%, 97%, 98%, 99% or more of identity with SEQ ID NO: 14, SEQ ID NO: 15, SEQ ID NO: 16 or SEQ ID NO: 20 and/or SEQ ID NO: 17, SEQ ID NO: 18, SEQ ID NO: 19 or SEQ ID NO: 21, respectively.

In one embodiment, the HCVR with SEQ ID NO: 14, SEQ ID NO: 15, SEQ ID NO: 16, SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47 and/or the LCVR with SEQ ID NO: 17, SEQ ID NO: 18, SEQ ID NO: 19, SEQ ID NO: 21, SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59 can be characterized as having an amino acid sequence that shares at least 70%, 75%, 80%, 85%, 90%, 95%, 96%, 97%, 98%, 99% or more of identity with SEQ ID NO: 14, SEQ ID NO: 15, SEQ ID NO: 16 or SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47 and/or SEQ ID NO: 17, SEQ ID NO: 18, SEQ ID NO: 19, SEQ ID NO: 21, SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59, respectively.

In one embodiment, the LCVR and/or the HCVR further comprises a leader sequence, preferably located N terminally from the LCVR amino acid sequence or N terminally from the HCVR amino acid sequence respectively. Examples of leader sequences include, but are not limited to, SEQ ID NO: 22 and 23.

SEQ ID NO: 22
MDIRLSLAFLVLFIKGVQC
SEQ ID NO: 23
MAAVQLLGLLLLWLPAMRC

In one embodiment, the LCVR comprises an amino acid sequence leader sequence SEQ ID NO: 22 located N terminally from the HCVR amino acid sequence (such as, for example, SEQ ID NO: 14, SEQ ID NO: 15, SEQ ID NO: 16, SEQ ID NO: 20, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46 or SEQ ID NO: 47).

In one embodiment, the LCVR comprises an amino acid leader sequence SEQ ID NO: 23 located N terminally from the LCVR amino acid sequence (such as, for example, SEQ ID NO: 17, SEQ ID NO: 18, SEQ ID NO: 19, SEQ ID NO: 21, SEQ ID NO: 36, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58 or SEQ ID NO: 59).

In one embodiment, an antibody of the present invention comprising the sequences SEQ ID NO: 14 and SEQ ID NO: 17 is herein referred as the Ald25H1 antibody.

The present invention further relates to an Ald25H1-like antibody, i.e., to an antibody binding the same epitope as Ald25H1, or substantially the same epitope than Ald25H1.

The present invention thus further relates to an antibody competing with Ald25H1 for binding to CD25.

In one embodiment, an antibody of the present invention comprising the sequences SEQ ID NO: 15 and SEQ ID NO: 18 is herein referred as the Ald25H2 antibody.

The present invention further relates to a Ald25H2-like antibody, i.e., to an antibody binding the same epitope as Ald25H2, or substantially the same epitope than Ald25H2. The present invention thus further relates to an antibody competing with Ald25H2 for binding to CD25.

In one embodiment, an antibody of the present invention comprising the sequences SEQ ID NO: 16 and SEQ ID NO: 19 is herein referred as the Ald25H4 antibody.

The present invention further relates to a Ald25H4 -like antibody, i.e., to an antibody binding the same epitope as Ald25H4, or substantially the same epitope than Ald25H4. The present invention thus further relates to an antibody competing with Ald25H4 for binding to CD25.

In one embodiment, the antibody or antigen-binding fragment thereof according to the present invention comprises a fully or substantially fully human heavy chain constant region (abbreviated herein as HCCR or C_{H}) and/or light chain constant region (abbreviated herein as LCCR or C_{L}).

In one embodiment, the constant region is of human origin.

In one embodiment, the antibody or antigen-binding fragment thereof according to the present invention comprises a fully or substantially fully murine HCCR and/or LCCR.

In one embodiment, the constant region is of murine origin.

In one embodiment, the antibody or antigen-binding fragment thereof according to the present invention is a murine antibody or fragment thereof.

In one embodiment, the antibody or antigen-binding fragment thereof according to the present invention is a chimeric antibody or fragment thereof.

A “chimeric antibody”, as used herein, refers to an antibody or antigen-binding fragment thereof comprising a first amino acid sequence linked to a second amino acid sequence with which it is not naturally linked in nature. The amino acid sequences may normally exist in separate proteins that are brought together in the fusion protein or they may normally exist in the same protein but are placed in a new arrangement in the fusion protein. A chimeric protein may be created, for example, by chemical synthesis, or by creating and translating a polynucleotide in which the peptide regions are encoded in the desired relationship. The term “chimeric antibody” encompasses herein antibodies and antigen-binding fragment thereof in which
(a) the constant region, or a portion thereof, is altered, replaced or exchanged so that the variable region is linked to a constant region of a different or altered class, effector function and/or species, or an entirely different molecule which confers new properties to the chimeric antibody, e.g., an enzyme, toxin, hormone, growth factor, drug, etc.; or
(b) the variable region, or a portion thereof, is altered, replaced or exchanged with a variable region, or portion thereof, having a different or altered antigen specificity; or with corresponding sequences from another species or from another antibody class or subclass.

In one embodiment, the antibody or antigen-binding fragment thereof according to the present invention is a humanized antibody or fragment thereof.

A “humanized antibody”, as used herein, refers to a chimeric antibody or antigen-binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or antigen-binding fragment thereof according to the present invention may include amino acid residues not encoded by human germline immunoglobulin sequences (e.g., mutations introduced by random or site-specific mutagenesis in vitro or by somatic mutation in vivo), for example in the CDRs. The term “humanized antibody” also includes antibodies and antigen-binding fragment thereof in which CDR sequences derived from the germline of another mammalian species, such as a mouse, have been grafted onto human framework sequences. In other words, the term “humanized antibody” refers to an antibody or antigen-binding fragment thereof in which the CDRs of a recipient human antibody are replaced by CDRs from a donor non-human antibody. Humanized antibodies or antigen-binding fragments thereof may also comprise residues of donor origin in the framework sequences. The humanized antibody or antigen-binding fragment thereof can also comprise at least a portion of a human immunoglobulin constant region. Humanized antibodies and or antigen-binding fragments thereof may also comprise residues which are found neither in the recipient antibody nor in the imported CDR or framework sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992. Curr Opin Biotechnol. 3(4):394-8; U.S. Pat. No. 4,816,567), including techniques such as “superhumanizing” antibodies (e.g., Tan et al., 2002. J Immunol. 169(2):1119-25) and “resurfacing” (e.g., Staelens et al., 2006. Mol Immunol. 43(8):1243-57; Roguska et al., 1994. Proc Nall Acad Sci USA. 91(3):969-73).

A “humanized antibody” retains a similar antigenic specificity as the original antibody.

However, using certain methods of humanization, the affinity and/or specificity of binding of the antibody may be increased.

Methods for humanizing the antibody or antigen-binding fragment thereof according to the present invention are well-known in the art. The choice of human variable domains, both light and heavy, to be used in making the humanized antibody or antigen-binding fragment thereof is very important to reduce antigenicity. According to the so-called “best-fit” method, the sequence of the variable domain of an antibody or antigen-binding fragment thereof according to the present invention is screened against the entire library of known human variable-domain sequences. The human sequence that is closest to the mouse sequence is then accepted as the human framework (FR) for the humanized antibody (Sims et al., 1993. J Immunol. 151(4):2296-308; Chothia & Lesk, 1987. J Mol Biol. 196(4):901-17).

Another method for humanizing the antibody or antigen-binding fragment thereof according to the present invention uses a particular framework from the consensus sequence of all human antibodies of a particular subgroup of light or heavy chains. The same framework can be used for several different humanized antibodies (Carter et al., 1992. Proc Nall Acad Sci USA. 89(10):4285-9; Presta et al., 1993. J Immunol. 151(5):2623-32). It is further important that antibodies be humanized with retention of high affinity for hCD25 and other favorable biological properties. To achieve this goal, according to a preferred method, humanized antibodies and antigen-binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for hCD25, is achieved. In general, the CDR residues are directly and most substantially involved in influencing antigen binding.

Another method for humanizing the antibody or antigen-binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized Examples of such transgenic or transchromosomic animal include, without limitation:
the XenoMouse (Abgenix, Fremont, Calif.), described in U.S. Pat. Nos. 5,939,598, 6,075,181, 6,114,598, 6,150,584 and 6,162,963;
the HuMAb Mouse® (Medarex, Inc.), described in Lonberg et al., 1994. Nature. 368(6474):856-859; Lonberg & Huszar, 1995. Int Rev Immunol. 13(1):65-93; Harding & Lonberg, 1995. Ann N Y Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7):845-51;
the KM Mouse®, described in Patent application WO2002043478;
the TC mice, described in Tomizuka et al., 2000. Proc Natl Acad Sci USA. 97(2):722-7; and
the OmniRat™ (OMT, Inc.), described in Patent application WO2008151081; Geurts et al., 2009. Science. 325(5939):433; Menoret et al., 2010. Eur J Immunol. 40(10):2932-41.

Humanized antibodies and antigen-binding fragments thereof may also be produced according to various other techniques, such as by using, for immunization, other transgenic animals that have been engineered to express a human antibody repertoire (Jakobovitz et al., 1993. Nature. 362(6417):255-8), or by selection of antibody repertoires using phage display methods. Such techniques are known to the skilled person and can be implemented starting from monoclonal antibodies or antigen-binding fragments thereof as disclosed in the present application.

In some embodiments, the antibody or antigen-binding fragment thereof according to the present invention comprising HCVR and LCVR (or CDRs thereof) may comprise a first constant domain (C_{H}1 and/or C_{L}), the amino acid sequence of which is fully or substantially human.

In one embodiment, the antibody or antigen-binding fragment thereof according to the present invention is a fully or substantially human antibody or fragment thereof.

In some embodiment, especially when the antibody or antigen-binding fragment thereof according to the present invention is intended for human therapeutic uses, it is typical for the entire constant region, or at least a part thereof, to have a fully or substantially human amino acid sequence. Therefore, one or more of, or any combination of, the C_{H}1 domain, hinge region, C_{H}2 domain, C_{H}3 domain and C_{L} domain (and C_{H}4 domain if present) may be fully or substantially human with respect to its amino acid sequence. Advantageously, the C_{H}1 domain, hinge region, C_{H}2 domain, C_{H}3 domain and C_{L} domain (and C_{H}4 domain if present) may all have a fully or substantially human amino acid sequence.

The term “substantially human”, in the context of the constant region of a humanized or chimeric antibody or antigen-binding fragment thereof, refers to an amino acid sequence identity of at least 70%, preferably at least 75%, 80%, 85%, 90%, 95%, 96%, 97%, 98%, 99% or more with a human constant region.

The term “human amino acid sequence”, in this context, refers to an amino acid sequence which is encoded by a human immunoglobulin gene, which includes germline, rearranged and somatically mutated genes. The present invention also contemplates proteins comprising constant domains of “human” sequence which have been altered, by one or more amino acid additions, deletions or substitutions with respect to the human sequence, excepting those embodiments where the presence of a “fully human hinge region” is expressly required.

The presence of a “fully human hinge region” in the antibody or antigen-binding fragment thereof according to the present invention may be beneficial both to minimize immunogenicity and to optimize stability of the antibody. It is considered that one or more amino acid substitutions, insertions or deletions may be made within the constant region of the heavy and/or the light chain, particularly within the Fc region Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody or antigen-binding fragment thereof, it may be desirable to modify the antibody or antigen-binding fragment thereof according to the present invention with respect to its binding properties to Fc receptors, for example to modulate effector function. For example, cysteine residue(s) may be introduced in the Fc region, thereby allowing interchain disulfide bond formation in this region. The homodimeric antibody thus generated may have improved effector function (Caron et al., 1992. J Exp Med. 176(4):1191-5; Shopes, 1992. J Immunol. 148(9):2918-22).

In one embodiment, the antibody or antigen-binding fragment thereof according to the present invention is from the IgG class.

In one embodiment, the antibody or antigen-binding fragment thereof according to the present invention is from the human IgG1 subclass. In another embodiment, the antibody or antigen-binding fragment thereof according to the present invention is from the human IgG2 subclass.

The Fc region of IgG antibodies interacts with cellular Fcγ receptors (FcγR) to stimulate and regulate downstream effector mechanisms. There are five activating receptors, namely FcγRI (CD64), FcγRIIa (CD32a), FcγRIIc (CD32c), FcγRIIIa (CD16a) and FcγRIIIb (CD16b), and one inhibitory receptor FcγRIIb (CD32b). The communication of IgG antibodies with the immune system is controlled and mediated by FcγRs, which relay the information sensed and gathered by antibodies to the immune system, providing a link between the innate and adaptive immune systems, and particularly in the context of biotherapeutics (Hayes J et al., 2016. J Inflamm Res 9: 209-219).

IgG subclasses vary in their ability to bind to FcγR and this differential binding determines their ability to elicit a range of functional responses. For example, in humans, FcγRIIIa is the major receptor involved in the activation of antibody-dependent cell-mediated cytotoxicity (ADCC) and IgG3 (followed closely by IgG1) display the highest affinities for this receptor, reflecting their ability to potently induce ADCC. IgG2 have been shown to have weak binding for this receptor.

In one embodiment, the antibody or antigen-binding fragment thereof according to the present invention binds FcγR with high affinity, preferably binds an activating receptor with high affinity.

In one embodiment, the antibody or antigen-binding fragment thereof according to the present invention binds FcγRI and/or FcγRIIa and/or FcγRIIc and/or FcγRIIIa and/or FcγRIIIb with high affinity.

In one embodiment, the IgG1 antibody binds to at least one Fc activating receptor. For example, the antibody may bind to one or more receptor selected from FcγRI, FcγRIIa, FcγRIIc, FcγRIIIa and FcγRIIIb. In one embodiment, the antibody is capable of binding to FcγRIIIa. In one embodiment, the antibody is capable of binding to FcγRIIa. In one embodiment, the antibody is capable of binding to FcγRIIIa, FcγRIIc and optionally FcγRI. In one embodiment, the antibody is capable of binding to FcγRIIIa, FcγRIIa and optionally FcγRI.

In one embodiment, the antibody or antigen-binding fragment thereof according to the present invention binds to at least one activating Fcγ receptor with a dissociation constant of less than about 10⁻⁶M, 10⁻⁷M, 10⁻⁸M, 10⁻⁹M or 10⁻¹⁰M.

In one embodiment, the IgG1 antibody binds to FcγRI, FcγRIIa, FcγRIIc, FcγRIIIa, and/or FcγRIIIb with a higher affinity than it binds to FcγRIIb, with low affinity.

In one embodiment, the antibody or antigen-binding fragment thereof according to the present invention is an IgG1 antibody, preferably a human IgG1 antibody.

In one embodiment, the antibody or antigen-binding fragment thereof according to the present invention may comprise human heavy chain constant regions sequences and allow to target, block, and/or deplete CD25-expressing cells to which they are bound.

In one embodiment, the proteins according to the present invention deplete CD25-expressing cells to which they are bound. In one embodiment, the proteins according to the present invention deplete Tregs to which they are bound. In one embodiment, the proteins according to the present invention also deplete or reduce tumor infiltrating regulatory T cells to which they are bound.

In one embodiment, the antibody or antigen-binding fragment thereof according to the present invention depletes CD25-expressing cells to which it is bound. In one embodiment, the antibody or antigen-binding fragment thereof according to the present invention depletes Tregs to which it is bound. In one embodiment, the antibody or antigen-binding fragment thereof according to the present invention also depletes or reduces tumor infiltrating regulatory T cells to which it is bound.

The term “deplete” or “depleting”, with respect to CD25-expressing cells or Tregs refers to the killing, elimination, lysis or induction of such killing, elimination or lysis, so as to negatively affect the number of CD25 expressing cells present in a sample or in a subject. In one embodiment, the antibody or antigen binding fragment thereof according to the present invention allows targeting, blocking proliferation, and/or depleting CD25-expressing cells or Treg cells. In one embodiment, the depletion is via ADCC. In one embodiment, the depletion is via ADCP. In one embodiment, the depletion is via CDC.

Thus, in one embodiment, the antibody of the present invention leads, directly or indirectly, to the depletion of CD25-expressing cells (e.g., leads to a 10%, 20%, 50%, 60%, 70%, 80%, 85% or greater elimination or decrease in number of CD25 expressing cells).

In one embodiment, the antibody or antigen-binding fragment thereof according to the present invention does not inhibit the binding of interleukin-2 (IL-2) to CD25 and depletes Tregs to which they are bound.

In one embodiment, the antibody or antigen-binding fragment thereof according to the present invention induces antibody dependent cellular cytotoxicity (ADCC).

The term “antibody-dependent cell-mediated cytotoxicity” or “ADCC” refers to a cell-mediated cytotoxicity induced in an antibody-dependent manner when the Fc region of said antibody bound to its antigen binds to the Fc receptor on effector cells such as natural killer cells, macrophages, neutrophils, eosinophils and mononuclear cells (e.g., peripheral blood mononuclear cells), thereby leading to lysis of the target cell. ADCC can be measured using assays that are known and available in the art (e.g., Clynes of a/. (1998) Proc Natl Acad Sci USA 95, 652-6).

In one embodiment, the antibody or antigen-binding fragment thereof according to the present invention is from the IgG1 subclass and has ADCC activity.

In one embodiment, the antibody or antigen-binding fragment thereof according to the present invention induces antibody-dependent cell-mediated phagocytosis (ADCP).

The term “Antibody-dependent cell-mediated phagocytosis” (ADCP) or “opsonisation” refers to a cell-mediated reaction in which nonspecific cytotoxic cells (e.g., phagocytes, macrophages) that express Fc receptors (FcRs) recognize antibody bound on a target cell and induce phagocytosis of the target cell. ADCP can be measured using assays that are known and available in the art (e.g., Clynes ef a/. (1998) Proc Natl Acad Sci USA 95, 652-6).

In one embodiment, the antibody or antigen-binding fragment thereof according to the present invention is from the IgG1 subclass and has ADCP activity.

In one embodiment, the antibody or antigen-binding fragment thereof according to the present invention induces complement-dependent cytotoxicity (CDC).

The term “Complement-dependent cytotoxicity” (CDC) refers to the induction of the lysis of antigen-expressing cells recognized by an antibody or antigen-binding fragment thereof of the invention in the presence of complement. The complement activation pathway is initiated by the binding of the first component of the complement system (C1q) to a molecule (e.g., an antibody) complexed with a cognate antigen. CDC can be measured using assays that are known and available in the art (e.g., Clynes ef a/. (1998) Proc Natl Acad Sci USA 95, 652-6; Gazzano-Santaro et al., J. Immunol. Methods, 202:163 (1996)).

In one embodiment, the antibody or antigen-binding fragment thereof according to the present invention is from the IgG1 subclass and has CDC activity.

The constant region of an antibody is important in the ability of an antibody to fix complement and mediate cell-dependent cytotoxicity and phagocytosis. Thus, as discussed herein, the isotype of an antibody may be selected on the basis of whether it is desirable for the antibody to mediate cytotoxicity/phagocytosis.

In one embodiment, the antibody or antigen-binding fragment thereof according to the present invention is linked to a toxic moiety.

In one embodiment, the antibody or antigen-binding fragment thereof according to the present invention is not conjugated, such as, for example, to a toxic moiety.

In one embodiment, the antibody or antigen-binding fragment thereof according to the present invention is not linked to a toxic moiety.

In one embodiment, the antibody or antigen-binding fragment thereof according to the present invention lacks an Fc domain (e.g., lacks a CH2 and/or CH3 domain) or comprises an Fc domain of IgG2 or IgG4 isotype.

In one embodiment, the antibody or antigen-binding fragment thereof according to the present invention does not comprise an Fc region that mediates ADCC, ADCP and/or CDC.

In one embodiment, the antibody or antigen-binding fragment thereof according to the present invention does not induce ADCC, ADCP and/or CDC.

Thus, in one embodiment, the antibody or antigen-binding fragment thereof according to the present invention does not lead, directly or indirectly, to the depletion CD25-expressing cells (e.g., do not lead to a 10%, 20%, 50%, 60% or greater elimination or decrease in number of CD25 cells). For example, the antibody of the present invention does not comprise an Fc domain capable of substantially binding to an FcγRIIIA (CD16) polypeptide.

In one embodiment, the antibody or antigen-binding fragment thereof according to the present invention is an engineered antibody or fragment thereof.

Engineered antibodies of the present invention include those in which modifications have been made to framework residues within VH and/or VL, e.g., to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis or PCR-mediated mutagenesis. Such “back-mutated” antibodies are also intended to be encompassed by the invention. Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Patent Publication No. 20030153043 by Carr et al.

In one embodiment, the antibody or antigen-binding fragment thereof according to the present invention is engineered to elicit an enhanced, increased or improved ADCC, ADCP, and/or CDC response.

As used herein, the term “enhanced, increased or improved ADCC, ADCP, and/or CDC response.” is relative to the ADCC, ADCP, and/or CDC response induced by the antibody or fragment thereof according to the invention as compared the ADCC, ADCP, and/or CDC response induced with other anti-CD25 antibodies, including those that do not inhibit the binding of interleukin 2 to CD25 and, for example unmodified anti-CD25 monoclonal antibodies.

Methods to increase ADCC, ADCP and/or CDC are well known in the art. For example, ADCC may be increased by methods that eliminate the fucose moiety from the antibody glycan, such as by production of the antibody in a YB2/0 cell line, or though the introduction of specific mutations on the Fc portion of human lgG1 (e.g., S298A/E333A/K334A, S239D/I332E/A330L,G236A/S239D/A330L/I332E) (Lazar ef al. (2006) Proc Natl Acad Sci USA 103, 2005-2010; Smith et al. (2012) Proc Natl 25 Acad Sci USA 109, 6181-6). ADCP may also be increased by the introduction of specific mutations on the Fc portion of human lgG1 (Richards ef al. (2008) Mol Cancer Ther 7, 2517-27). CDC response may be increased with mutations in the antibody that increase the affinity of C1q binding (Idusogie ef a/. (2001) J Immunol 166, 2571-5).

Of note, methods to decrease or abolish ADCC, ADCP and/or CDC are also well known in the art. For example, ADCC may be decreased or abolished by methods modifying the glycosylation profile of the Fc domain of the immunoglobulin. CDC can be decreased or abolished by the replacement of one or more amino acids by other amino acid such that the antibody has altered C2q binding (U.S. Pat. No. 6,194,551 by Idusogie et al.).

In one embodiment, the antibody or antigen-binding fragment thereof according to the present invention is engineered to modify its glycosylation. For example, the antibody according to the invention is aglycosyled (i.e., the antibody lacks glycosylation). Glycosylation can be altered to, for example, increase the affinity of the antibody for the antigen or alter the ADCC activity of the antibody. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al (incorporated herein by reference). Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered fucosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP1176195 by Hang et al. (incorporated herein by reference) describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the antibody or antigen-binding fragment thereof of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta (incorporated herein by reference) describes a variant CHO cell line, Lec13 cells, with reduced ability to attach fucose to Asn(297)-linked carbohydrates, also resulting in hypofucosylation of antibodies expressed in that host cell (see also Shields, R. L. et al, 2002 J. Biol. Chem. 277:26733-26740). PCT Publication WO 99/54342 by Umana et al. (incorporated herein by reference) describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human antibody (preferably the monoclonal antibody) of the present invention can be produced in yeasts or filamentous fungi engineered for mammalian-like glycosylation pattern and capable of producing antibodies lacking fucose as glycosylation pattern (see for example EP1297172B 1).

In one embodiment, the antibody or antigen-binding fragment thereof according to the present invention is a pegylated antibody or fragment thereof.

An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (DY12-DY120) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the present invention, such as, for example, as described in EP0154316 (Nishimura et al.) and EP0401384 (Ishikawa et al.).

The present invention further relates to a fusion protein comprising an antibody or antigen binding fragment thereof as described herein.

In one embodiment, said fusion protein comprises a second antigen binding moiety. In one embodiment, said fusion protein is a multispecific antibody, for example a bispecific antibody.

In one embodiment, the antibody or antigen-binding fragment thereof is bispecific, and is further capable of binding to another molecule.

In one embodiment, the other molecule is an immune receptor. Examples of immune receptors that may be bound by a bispecific antibody of the present invention include, but are not limited to, CTLA4, PD-1, PD-L1, TIM-3, LAG-3, B7H3, B7H4, B7H6, 4-1BB, OX40, ICOS, GITR, TIGIT, CD27-CD70, CD40, BTLA, HVEM, CD160 and CEACAM-1.

In one embodiment, the antibody or antigen-binding fragment thereof is bispecific, and is further capable of binding to a costimulatory molecule. Examples of costimulatory molecules include, but are not limited to, 4-1BB, ICOS, GITR, CD27-CD70, CD40 and OX40.

In one embodiment, the antibody or antigen-binding fragment thereof is bispecific, and is capable of binding to OX40. In one embodiment, the antibody or antigen-binding fragment thereof is bispecific, and is capable of binding to GITR. In one embodiment, the antibody or antigen-binding fragment thereof is bispecific, and is capable of binding to ICOS.

In another embodiment, the antibody or antigen-binding fragment thereof is bispecific, and is further capable of binding to a coinhibitory molecule. Examples of coinhibitory molecules include, but are not limited to, CTLA4, PD-1, PD-L1, TIM-3, LAG-3, TIGIT, BTLA, HVEM, CD160 and CEACAM-1.

In one embodiment, the antibody or antigen-binding fragment thereof is bispecific, and is capable of binding to CTLA4. In one embodiment, the antibody or antigen-binding fragment thereof is bispecific, and is capable of binding to PD-1. In one embodiment, the antibody or antigen-binding fragment thereof is bispecific, and is capable of binding to TIGIT.

In one embodiment, said fusion protein comprises a second antigen binding moiety that binds an immune checkpoint protein. Examples of immune checkpoint proteins (including checkpoint inhibitors and checkpoint agonists) are listed herein.

In one embodiment, said fusion protein comprises a second antigen binding moiety that binds a T cell marker, such as, for example, CD3 or CD28.

In one embodiment, said fusion protein comprises a second antigen binding moiety that binds a NK cell marker, such as, for example, an activating NK receptor. Examples of activating NK receptors include, but are not limited to, activating forms of KIR proteins (for example KIR2DS proteins), CD160-TM, NKG2D, IL-2R, IL-12R, IL-15R, IL-18R and IL-21R.

In one embodiment, the antibody or antigen-binding fragment thereof is conjugated with a therapeutic moiety, i.e., a drug. The therapeutic moiety can be, e.g., a chemotherapeutic agent, an immunosuppressant, a lytic peptide, a radionuclide or a toxin.

In another embodiment, the antibody or antigen-binding fragment thereof is not conjugated with a radionuclide (i.e., the antibody or antigen-binding fragment thereof is not radiolabeled) and/or with a toxin.

Examples of radionuclides include, but are not limited to, ⁹⁰Y, ¹³¹L or ⁶⁷Cu.

Examples of toxins include, but are not limited to, doxorubicin and calicheamicin.

In another embodiment, the antibody or antigen binding fragment is conjugated with a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, momordica charantia inhibitor, curcin, crotin, sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target humanepidermal growth factor receptor 2-positive breast cancer. Clin. Cancer Res.16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

Another object of the invention is an isolated nucleic acid encoding the antibody or antigen-binding fragment thereof binding to human CD25 according to the present invention. Another object of the invention is an isolated nucleic acid encoding the fusion protein according to the present invention.

An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid.

Of course, this refers to the nucleic acid as originally isolated and does not exclude genes or sequences later added to the isolated nucleic acid by the hand of man

In one embodiment, the isolated nucleic acid is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A_{260/280})), and most preferably more than 96%, 97%, 98% or 99% by weight; or
(2) homogeneity as shown by agarose gel electrophoresis and using an intercalating agent such as ethidium bromide, SYBR Green, GelGreen or the like.

In one embodiment, the nucleic acid encodes at least a heavy chain variable region or a light chain variable region of the antibody according to the present invention. In one embodiment, the nucleic acid may encode variable and constant regions of the antibody according to the present invention. In one embodiment, the nucleic acid may encode heavy and light chains of the antibody on separate nucleic acids or on the same nucleic acid molecule.

In one embodiment, the nucleic acid according to the present invention comprises or consists of a sequence encoding the HCVR of the antibody or antigen-binding fragment thereof according to the invention.

In one embodiment, the nucleic acid according to the present invention comprises or consists of a sequence SEQ ID NO: 24 or any sequence sharing at least 70%, 75%, 80%, 85%, 90%, 95%, 96%, 97%, 98%, 99% or more identity with SEQ ID NO: 24.

SEQ ID NO: 24
GAGGTGCAGCTGGTGGAX₁TCTGGGGGCGGCTTAGTGCAGCCTGGAAGGTC
CATGAAACTCTCCTGTGCAGX₂CTCAGGATTCX₃CTTTCAGTX₄ACCATGCC
ATGGCCTGGGTCCGCCAGGCTCCAAAGAAGGGTCTGX₅AGTGGGTCGCATA
CATTAGTTATGATGGGATAACACTTACTATCGAGACTCCGTGAAGGGCCGA
TTCACTATCTCCAGAGATAATGCAX₆X₇AAGLACCCTATX₉CCTGCAAATX₁₀
GACAGTCTGAGGTCTGAGGACACGGCCACTTATTAX₁₁TGTACAACAGGGG
GTAATTCGGGGTACGACTGGGGCCAAGGAGTCATGGTCACAGTCTCCTCA

wherein X₁ is G or A, X₂ is C or T, X₃ is C or A, X₄ is G or A, X₅ is C or G, X₆ is A or C, X₇ is G or A, X₈ is T or C, X₉ is T or A, X₁₀ is G or T and X₁₁ is C or T.

In one embodiment, the nucleic acid according to the present invention comprises or consists of a sequence encoding the LCVR of the antibody or antigen-binding fragment thereof according to the invention.

In one embodiment, the nucleic acid according to the present invention comprises or consists of a sequence SEQ ID NO: 25 or any sequence sharing at least 70%, 75%, 80%, 85%, 90%, 95%, 96%, 97%, 98%, 99% or more identity with SEQ ID NO: 25.

SEQ ID NO: 25
GACATCCAGATGACCCAGTCTCCTTCATTCCTGTCTGCATCTGTGGGAGA
CAGAGTCACX₁ATCAACTGCAAAGX₂AAGTCAGAATGTTAACAAGTTCX₃T
AAACTGGTATCAGCAAAAGCTTGGAGAAGCTCCCAGACGCCTGATCTATG
GTACAAACAGTTTGCAAACCGGCATCCCATCAAGGTTCAGTGGCAGTGGA
TCTGGX₄ACAGATTACACACTCACCATCAGCAGCCTGCAGCCTGAAGATGT
TGCCACGTATTTCTGCCAGCAGTATAX₅CAGTTGGCCGTGGACGTTCGGTG
GAGGCACCAAGCTGGAATTGAAA

wherein X₁ is C or T, X₂ is C or G, X₃ is T or G, X₄ is T or A and X₅ is G or C.

In one embodiment, the nucleic acid according to the present invention comprises or consists of:
a sequence encoding the HCVR of the antibody or antigen-binding fragment thereof according to the invention; and
a sequence encoding the LCVR of the antibody or antigen-binding fragment thereof according to the invention.

In one embodiment, the nucleic acid according to the present invention comprises or consists of:
a sequence SEQ ID NO: 24 or any sequence sharing at least 70%, 75%, 80%, 85%, 90%, 95%, 96%, 97%, 98%, 99% or more identity with SEQ ID NO: 24; and
a sequence SEQ ID NO: 25 or any sequence sharing at least 70%, 75%, 80%, 85%, 90%, 95%, 96%, 97%, 98%, 99% or more identity with SEQ ID NO: 25.

In one embodiment, the nucleic acid according to the present invention comprises or consists of a sequence encoding the HCVR of the antibody or antigen-binding fragment thereof according to the invention.

In one embodiment, the nucleic acid according to the present invention comprises or consists of a sequence SEQ ID NO: 26, SEQ ID NO: 27, SEQ ID NO: 28 or any sequence sharing at least 70%, 75%, 80%, 85%, 90%, 95%, 96%, 97%, 98%, 99% or more identity with SEQ ID NO: 26, SEQ ID NO: 27 or SEQ ID NO: 28.

SEQ ID NO: 26
GAGGTGCAGCTGGTGGAGTCTGGGGGCGGCTTAGTGCAGCCTGGAAGGTCC
ATGAAACTCTCCTGTGCAGCCTCAGGATTCCCTTTCAGTGACCATGCCATG
GCCTGGGTCCGCCAGGCTCCAAAGAAGGGTCTGCAGTGGGTCGCATACATT
AGTTATGATGGGGATAACACTTACTATCGAGACTCCGTGAAGGGCCGATTC
ACTATCTCCAGAGATAATGCAAGAAGTACCCTATTCCTGCAAATGGACAGT
CTGAGGTCTGAGGACACGGCCACTTATTATTGTACAACAGGGGGTAATTCG
GGGTACGACTGGGGCCAAGGAGTCATGGTCACAGTCTCCTCA
SEQ ID NO: 27
GAGGTGCAGCTGGTGGAATCTGGGGGCGGCTTAGTGCAGCCTGGAAGGTCC
ATGAAACTCTCCTGTGCAGCCTCAGGATTCACTTTCAGTAACCATGCCATG
GCCTGGGTCCGCCAGGCTCCAAAGAAGGGTCTGGAGTGGGTCGCATACATT
AGTTATGATGGGGATAACACTTACTATCGAGACTCCGTGAAGGGCCGATTC
ACTATCTCCAGAGATAATGCAAAAAGCACCCTATACCTGCAAATTGACAGT
CTGAGGTCTGAGGACACGGCCACTTATTACTGTACAACAGGGGGTAATTCG
GGGTACGACTGGGGCCAAGGAGTCATGGTCACAGTCTCCTCA
SEQ ID NO: 28
GAGGTGCAGCTGGTGGAGTCTGGGGGCGGCTTAGTGCAGCCTGGAAGGTCC
ATGAAACTCTCCTGTGCAGTCTCAGGATTCACTTTCAGTAACCATGCCATG
GCCTGGGTCCGCCAGGCTCCAAAGAAGGGTCTGGAGTGGGTCGCATACATT
AGTTATGATGGGGATAACACTTACTATCGAGACTCCGTGAAGGGCCGATTC
ACTATCTCCAGAGATAATGCACAAAGCACCCTATACCTGCAAATGGACAGT
CTGAGGTCTGAGGACACGGCCACTTATTACTGTACAACAGGGGGTAATTCG
GGGTACGACTGGGGCCAAGGAGTCATGGTCACAGTCTCCTCA

In one embodiment, the nucleic acid according to the present invention comprises or consists of a sequence encoding the LCVR of the antibody or antigen-binding fragment thereof according to the invention.

In one embodiment, the nucleic acid according to the present invention comprises or consists of a sequence SEQ ID NO: 29, SEQ ID NO: 30, SEQ ID NO: 31 or any sequence sharing at least 70%, 75%, 80%, 85%, 90%, 95%, 96%, 97%, 98%, 99% or more identity with SEQ ID NO: 29, SEQ ID NO: 30 or SEQ ID NO: 31.

SEQ ID NO: 29
GACATCCAGATGACCCAGTCTCCTTCATTCCTGTCTGCATCTGTGGGAGAC
AGAGTCACCATCAACTGCAAAGCAAGTCAGAATGTTAACAAGTTCTTAAAC
TGGTATCAGCAAAAGCTTGGAGAAGCTCCCAGACGCCTGATCTATGGTACA
AACAGTTTGCAAACCGGCATCCCATCAAGGTTCAGTGGCAGTGGATCTGGT
ACAGATTACACACTCACCATCAGCAGCCTGCAGCCTGAAGATGTTGCCACG
TATTTCTGCCAGCAGTATAGCAGTTGGCCGTGGACGTTCGGTGGAGGCACC
AAGCTGGAATTGAAA
SEQ ID NO: 30
GACATCCAGATGACCCAGTCTCCTTCATTCCTGTCTGCATCTGTGGGAGAC
AGAGTCACTATCAACTGCAAAGCAAGTCAGAATGTTAACAAGTTCGTAAAC
TGGTATCAGCAAAAGCTTGGAGAAGCTCCCAGACGCCTGATCTATGGTACA
AACAGTTTGCAAACCGGCATCCCATCAAGGTTCAGTGGCAGTGGATCTGGA
ACAGATTACACACTCACCATCAGCAGCCTGCAGCCTGAAGATGTTGCCACT
GATTTCTGCCAGCAGTATAGCAGTTGGCCGTGGACGTTCGGTGGAGGCACC
AAGCTGGAATTGAAA
SEQ ID NO: 31
GACATCCAGATGACCCAGTCTCCTTCATTCCTGTCTGCATCTGTGGGAGAC
AGAGTCACTATCAACTGCAAAGGAAGTCAGAATGTTAACAAGTTCTTAAAC
TGGTATCAGCAAAAGCTTGGAGAAGCTCCCAGACGCCTGATCTATGGTACA
AACAGTTTGCAAACCGGCATCCCATCAAGGTTCAGTGGCAGTGGATCTGGT
ACAGATTACACACTCACCATCAGCAGCCTGCAGCCTGAAGATGTTGCCACG
TATTTCTGCCAGCAGTATACCAGTTGGCCGTGGACGTTCGGTGGAGGCACC
AAGCTGGAATTGAAA

In one embodiment, the nucleic acid according to the present invention comprises or consists of:
a sequence encoding the HCVR of the antibody or antigen-binding fragment thereof according to the invention; and
a sequence encoding the LCVR of the antibody or antigen-binding fragment thereof according to the invention.

In one embodiment, the nucleic acid according to the present invention comprises or consists of:
a sequence SEQ ID NO: 26, SEQ ID NO: 27, SEQ ID NO: 28 or any sequence sharing at least 70%, 75%, 80%, 85%, 90%, 95%, 96%, 97%, 98%, 99% or more identity with SEQ ID NO: 26, SEQ ID NO: 27 or SEQ ID NO: 28; and
a sequence SEQ ID NO: 29, SEQ ID NO: 30, SEQ ID NO: 31 or any sequence sharing at least 70%, 75%, 80%, 85%, 90%, 95%, 96%, 97%, 98%, 99% or more identity with SEQ ID NO: 29, SEQ ID NO: 30 or SEQ ID NO: 31.

In one embodiment, the nucleic acid according to the present invention comprises or consists of:
a sequence encoding the HCVR comprising or consisting of the sequence SEQ ID NO: 26; and
a sequence encoding the LCVR comprising or consisting of the sequence

SEQ ID NO: 29.

In one embodiment, the nucleic acid according to the present invention comprises or consists of:
a sequence encoding the HCVR comprising or consisting of the sequence SEQ ID NO: 27; and
a sequence encoding the LCVR comprising or consisting of the sequence SEQ ID NO: 30.

In one embodiment, the nucleic acid according to the present invention comprises or consists of:
a sequence encoding the HCVR comprising or consisting of the sequence SEQ ID NO: 28; and
a sequence encoding the LCVR comprising or consisting of the sequence SEQ ID NO: 31.

In one embodiment, the HCVR and/or the LCVR further comprises a leader sequence, preferably located in the 5′ from the HCVR nucleic acid sequence or in the 5′ from the LCVR nucleic acid sequence, respectively. Examples of leader sequences include, but are not limited to, SEQ ID NO: 22 and 23, encoded respectively by SEQ ID NO: 32 and SEQ ID NO: 33.

SEQ ID NO: 32
ATGGACATCAGGCTCAGCTTGGCTTTCCTTGTCCTTTTCATAAAAGGTGT
CCAGTGT
SEQ ID NO: 33
ATGGCTGCAGTTCAACTCTTAGGGCTGCTGCTGCTTTGGCTCCCAGCCAT
GAGATGT

In one embodiment, the HCVR comprises a nucleic acid leader sequence SEQ ID NO: 32 located in the 5′ from the HCVR nucleic acid sequence (SEQ ID NO: 24, SEQ ID NO: 26, SEQ ID NO: 27 or SEQ ID NO: 28).

In one embodiment, the LCVR comprises a nucleic acid sequence leader sequence SEQ ID NO: 33 located in the 5′ from the HCVR nucleic acid sequence (SEQ ID NO: 25, SEQ ID NO: 29, SEQ ID NO: 30 or SEQ ID NO: 31).

In one embodiment, the nucleic acid according to the present invention comprises or consists of a sequence encoding a fully or substantially fully human HCCR and/or LCCR of the antibody or antigen-binding fragment thereof according to the invention.

In such embodiment, constant regions may be derived from any human antibody constant regions.

In one embodiment, the nucleic acid according to the present invention comprises or consists of a sequence encoding a fully or substantially fully murine HCCR and/or LCCR of the antibody or antigen-binding fragment thereof according to the invention.

In such embodiment, constant regions may be derived from any murine antibody constant regions.

In one embodiment, the nucleic acid according to the present invention comprises or consists of a sequence encoding the heavy chain of the chimeric antibody or antigen-binding fragment thereof according to the invention.

In one embodiment, the nucleic acid according to the present invention comprises or consists of a sequence encoding the light chain of the chimeric antibody or antigen-binding fragment thereof according to the invention.

In one embodiment, the nucleic acid according to the present invention comprises or consists of a sequence encoding the heavy chain of the humanized antibody or antigen-binding fragment thereof according to the invention.

In one embodiment, the nucleic acid according to the present invention comprises or consists of a sequence encoding the light chain of the humanized antibody or antigen-binding fragment thereof according to the invention.

Typically, said nucleic acid is a DNA or RNA molecule, which may be included in any suitable vector, such as for example plasmid, cosimd, episome, artificial chromosome, phage or a viral vector.

Thus, another object of the present invention is an expression vector comprising a nucleic acid encoding the antibody or antigen-binding fragment thereof binding to human CD25 according to the present invention. Another object of the present invention is an expression vector comprising a nucleic acid encoding a fusion protein according to the present invention.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

In one embodiment, the expression vector according to the present invention comprises a sequence encoding the HCVR of the antibody or antigen-binding fragment thereof according to the invention, operably linked to regulatory elements.

In one embodiment, the expression vector according to the present invention comprises a sequence SEQ ID NO: 24, SEQ ID NO: 26, SEQ ID NO: 27, SEQ ID NO: 28 or any sequence sharing at least 70%, 75%, 80%, 85%, 90%, 95%, 96%, 97%, 98%, 99% or more identity with SEQ ID NO: 24, SEQ ID NO: 26, SEQ ID NO: 27or SEQ ID NO: 28, operably linked to regulatory elements.

In one embodiment, the expression vector according to the present invention comprises a sequence encoding the LCVR of the antibody or antigen-binding fragment thereof according to the invention, operably linked to regulatory elements.

In one embodiment, the expression vector according to the present invention comprises a sequence SEQ ID NO: 25, SEQ ID NO: 29, SEQ ID NO: 30, SEQ ID NO: 31 or any sequence sharing at least 70%, 75%, 80%, 85%, 90%, 95%, 96%, 97%, 98%, 99% or more identity with SEQ ID NO: 25, SEQ ID NO: 29, SEQ ID NO: 30 or SEQ ID NO: 31, operably linked to regulatory elements.

In one embodiment, the expression vector according to the present invention comprises:
a sequence encoding the HCVR of the antibody or antigen-binding fragment thereof according to the invention, operably linked to regulatory elements; and
a sequence encoding the LCVR of the antibody or antigen-binding fragment thereof according to the invention, operably linked to regulatory elements.

In one embodiment, the expression vector according to the present invention comprises:
a sequence encoding the HCVR comprising or consisting of the sequence SEQ ID NO: 24, SEQ ID NO: 26, SEQ ID NO: 27, SEQ ID NO: 28 or any sequence sharing at least 70%, 75%, 80%, 85%, 90%, 95%, 96%, 97%, 98%, 99% or more identity with SEQ ID NO: 24, SEQ ID NO: 26, SEQ ID NO: 27 or SEQ ID NO: 28; and
a sequence encoding the LCVR comprising or consisting of the sequence SEQ ID NO: 25, SEQ ID NO: 29, SEQ ID NO: 30, SEQ ID NO: 31 or any sequence sharing at least 70%, 75%, 80%, 85%, 90%, 95%, 96%, 97%, 98%, 99% or more identity with SEQ ID NO: 29, SEQ ID NO: 30 or SEQ ID NO: 31.

In one embodiment, the expression vector according to the present invention comprises:
a sequence encoding the HCVR comprising or consisting of the sequence SEQ ID NO: 24; and
a sequence encoding the LCVR comprising or consisting of the sequence SEQ ID NO: 25.

In one embodiment, the expression vector according to the present invention comprises:
a sequence encoding the HCVR comprising or consisting of the sequence SEQ ID NO: 26; and
a sequence encoding the LCVR comprising or consisting of the sequence SEQ ID NO: 29.

In one embodiment, the nucleic acid according to the present invention comprises or consists of:
a sequence encoding the HCVR comprising or consisting of the sequence SEQ ID NO: 27; and
a sequence encoding the LCVR comprising or consisting of the sequence SEQ ID NO: 30.

In one embodiment, the nucleic acid according to the present invention comprises or consists of:
a sequence encoding the HCVR comprising or consisting of the sequence SEQ ID NO: 28; and
a sequence encoding the LCVR comprising or consisting of the sequence SEQ ID NO: 31.

In one embodiment, the expression vector according to the present invention comprises a sequence encoding the HCCR of the antibody or antigen-binding fragment thereof according to the invention, operably linked to regulatory elements, wherein said HCCR may be derived from any human antibody HCCR.

In one embodiment, the expression vector according to the present invention comprises a sequence encoding the LCCR of the antibody or antigen-binding fragment thereof according to the invention, operably linked to regulatory elements, wherein said LCCR may be derived from any human antibody LCCR.

In one embodiment, the expression vector according to the present invention comprises a sequence encoding the HCCR of the antibody or antigen-binding fragment thereof according to the invention, operably linked to regulatory elements, wherein said HCCR may be derived from any murine antibody HCCR.

In one embodiment, the expression vector according to the present invention comprises a sequence encoding the LCCR of the antibody or antigen-binding fragment thereof according to the invention, operably linked to regulatory elements, wherein said LCCR may be derived from any murine antibody LCCR.

In one embodiment, the expression vector according to the present invention comprises a sequence encoding the heavy chain of the chimeric antibody or antigen-binding fragment thereof according to the invention, operably linked to regulatory elements.

In one embodiment, the expression vector according to the present invention comprises a sequence encoding the light chain of the chimeric antibody or antigen-binding fragment thereof according to the invention, operably linked to regulatory elements.

In one embodiment, the expression vector according to the present invention comprises a sequence encoding the heavy chain of the humanized antibody or antigen-binding fragment thereof according to the invention, operably linked to regulatory elements.

In one embodiment, the expression vector according to the present invention comprises a sequence encoding the light chain of the humanized antibody or antigen-binding fragment thereof according to the invention, operably linked to regulatory elements.

In one embodiment, the expression vector according to the present invention is monocistronic.

By “monocistronic”, it is meant that a single nucleic acid is expressed in a single expression vector.

In one embodiment, the expression vector according to the present invention is polycistronic.

By “polycistronic”, it is meant that at least two or more nucleic acids are expressed in a single expression vector.

Another object of the invention is an isolated host cell comprising said vector. Said host cell may be used for the recombinant production of the antibodies of the invention.

In an embodiment, host cells may be prokaryote, yeast, or eukaryote cells, preferably mammalian cells, such as, for example: monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art. It should be noted that the term “host cell” generally refers to a cultured cell line. Whole human beings into which an expression vector encoding an antigen binding protein according to the invention has been introduced are explicitly excluded from the definition of a “host cell”.

Another object of the present invention is a method of producing and purifying the isolated antibody or an antigen-binding fragment thereof, binding to human CD25 (hCD25) according to the present invention.

In one embodiment, the method comprises:
introducing in vitro or ex vivo a recombinant nucleic acid or a vector as described above into a competent host cell;
culturing in vitro or ex vivo host cells transformed with the nucleic acid or expression vector according to the present invention, under conditions suitable for expression of the antibody or antigen-binding fragment thereof;
optionally, selecting the cells which express and/or secrete said antibody; and
recovering the expressed antibody or antigen-binding fragment thereof.

This recombinant process can be used for large scale production of antibodies or antigen-binding fragments thereof, including monoclonal antibodies intended for in vitro, ex vivo and/or in vivo therapeutic and/or diagnostic uses.

These processes are well-known in the art (Subramanian (Ed.), 2004. Antibodies (1st ed., Vol. 1: Production and Purification). New York, N.Y.: Springer US).

In an embodiment, the expressed antibody or antigen-binding fragment thereof is further purified.

Methods to purify the antibody or antigen-binding fragment thereof according to the present invention are well-known in the art (Subramanian (Ed.), 2004. Antibodies (1st ed., Vol. 1: Production and Purification). New York, N.Y.: Springer US), and include, without limitation, protein A-Sepharose, gel electrophoresis, chromatography, preferably by affinity chromatography, more preferably by affinity chromatography on protein L agarose.

Another object of the present invention is a composition comprising, consisting essentially of or consisting of at least one protein binding to human CD25 (hCD25) according to the present invention.

Another object of the present invention is a composition comprising, consisting essentially of or consisting of at least one antibody binding to human CD25 (hCD25) according to the present invention or at least one antigen-binding fragment thereof.

Another object of the present invention is a composition comprising, consisting essentially of or consisting of at least one fusion protein according to the present invention.

A further object of the present invention is a composition comprising, consisting essentially of or consisting of at least one nucleic acid encoding an antibody binding to hCD25 according to the present invention or an antigen-binding fragment thereof or a fusion protein according to the present invention.

A further object of the present invention is a pharmaceutical composition comprising, consisting essentially of or consisting of at least one protein binding to hCD25 according to the present invention, and at least one pharmaceutically acceptable excipient.

Another object of the present invention is a pharmaceutical composition comprising, consisting essentially of or consisting of at least one antibody binding to human CD25 (hCD25) according to the present invention or at least one antigen-binding fragment thereof, and at least one pharmaceutically acceptable excipient.

Another object of the present invention is a pharmaceutical composition comprising, consisting essentially of or consisting of at least one fusion protein according to the present invention and at least one pharmaceutically acceptable excipient.

A further object of the present invention is a pharmaceutical composition comprising, consisting essentially of or consisting of at least one nucleic acid encoding an antibody binding to hCD25 according to the present invention or an antigen-binding fragment thereof, or a fusion protein according to the present invention, and at least one pharmaceutically acceptable excipient.

A further object of the present invention is a pharmaceutical composition comprising, consisting essentially of or consisting of at least one expression vector comprising at least one nucleic acid encoding an antibody binding to hCD25 according to the present invention or an antigen-binding fragment thereof, and at least one pharmaceutically acceptable excipient.

As used herein, “consisting essentially of” with reference to a composition, means that the at least one protein, antibody or antigen-binding fragment thereof, fusion protein, nucleic acid or expression vector is the only one therapeutic agent or agent with a biologic activity within said composition.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

A further object of the present invention is a medicament comprising, consisting essentially of or consisting of at least one protein binding to hCD25 according to the present invention.

A further object of the present invention is a medicament comprising, consisting essentially of or consisting of at least one antibody binding to hCD25 according to the present invention or at least one antigen-binding fragment thereof.

A further object of the present invention is a medicament comprising, consisting essentially of or consisting of at least one fusion protein according to the present invention.

A further object of the present invention is a medicament comprising, consisting essentially of or consisting of at least one nucleic acid encoding an antibody binding to hCD25 according to the present invention or an antigen-binding fragment thereof or a fusion protein according to the present invention.

A further object of the present invention is a medicament comprising, consisting essentially of or consisting of at least one expression vector comprising at least one nucleic acid encoding an antibody binding to human CD25 (hCD25) according to the present invention or an antigen-binding fragment thereof.

For use in administration to a subject, the composition, pharmaceutical composition or medicament will be formulated for administration to the subject. The composition, pharmaceutical composition or medicament according to the present invention may be administered parenterally, by inhalation spray, rectally, nasally, or via an implanted reservoir. The term administration used herein includes subcutaneous, intravenous, intramuscular, intra-articular, intra-synovial, intrasternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the isolated protein, the isolated antibody or antigen-binding fragment thereof, the fusion protein, nucleic acid, expression vector, composition, pharmaceutical composition or medicament according to the present invention is to be administered to the subject in need thereof in a therapeutically effective amount.

It will be however understood that the total daily usage of the isolated protein, isolated antibody or antigen-binding fragment thereof, fusion protein, nucleic acid, expression vector, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the isolated protein, isolated antibody or antigen-binding fragment thereof, fusion protein, nucleic acid, expression vector, composition, pharmaceutical composition or medicament employed; the age, body weight, general health, sex and diet of the subject; the time of administration, route of administration, and rate of excretion of the specific isolated protein, isolated antibody or antigen-binding fragment thereof, fusion protein, nucleic acid, expression vector, composition, pharmaceutical composition or medicament employed; the duration of the treatment; drugs used in combination or coincidental with the specific isolated protein, isolated antibody or antigen-binding fragment thereof, fusion protein, nucleic acid, expression vector, composition, pharmaceutical composition or medicament employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

Regimens or dosages used for administration of the protein, antibody or fusion protein can be adapted as function of various parameters, and in particular as a function of the mode of administration used, of the relevant pathology, or of the desired duration of treatment. For example, it is well within the skill of the art to start doses of the proteins, antibodies or fusion proteins at levels lower than those required to achieve the desired therapeutic effect and to gradually increase the dosage until the desired effect is achieved. The daily dosage of the proteins, antibodies or fusion proteins may be varied over a wide range from 0.01 to 1000 mg per adult per day. Compositions may contain 0.01, 0.05, 0.1, 0.5, 1.0, 2.5, 5.0, 10.0, 15.0, 25.0, 50.0, 100, 250, and 500 mg of the active ingredient for the symptomatic adjustment of the dosage to the subject to be treated. A pharmaceutical composition or medicament typically contains from about 0.01 mg to about 500mg of active ingredient. A therapeutically effective amount of the drug is ordinarily supplied at a dosage level from 0.0002 mg/kg to about 20 mg/kg of body weight per day. For example, a protein, an antibody, a fusion protein present in a composition, pharmaceutical composition or medicament of this invention can be supplied at a concentration ranging from 1 mg/mL to about 100 mg/mL, such as, for example, at a concentration of 1 mg/mL, 5 mg/mL, 10 mg/mL, 50 mg/mL or 100 mg/mL. In one embodiment, the protein, antibody or fusion protein is supplied at a concentration of about 10 mg/mL in either 100 mg (10 mL) or 500 mg (50 mL) single use -vials. It will be appreciated that these dosages are exemplary and that an optimal dosage can be adapted taking into account the affinity and tolerability of the particular antibody in the pharmaceutical composition that must be determined in clinical trials.

The present invention relates to at least one isolated protein as described herein, for treating (or for use in treating) diseases, disorders or symptoms in a subject in need thereof.

The present invention relates to at least one isolated antibody or antigen-binding fragment thereof, or of a composition or pharmaceutical composition, or a medicament as described herein, for treating (or for use in treating) diseases, disorders or symptoms in a subject in need thereof.

The present invention relates to at least one fusion protein as described herein, for treating (or for use in treating) diseases, disorders or symptoms in a subject in need thereof.

The present invention thus further relates to a method for treating diseases, disorders or symptoms in a subject in need thereof, comprising administering to the subject an antibody or antigen-binding fragment thereof, or a composition, a pharmaceutical composition, or a medicament as described herein.

The present invention thus further relates to a method for treating diseases, disorders or symptoms in a subject in need thereof, comprising administering to the subject an isolated protein or a fusion protein as described herein.

Examples of diseases that may be treated with the isolated protein, the antibody or fragment thereof or the fusion protein as described hereinabove, include, but are not limited to cancers and infectious diseases.

In one embodiment, the isolated protein, the antibody or antigen-binding fragment thereof or the fusion protein according to the present invention may be used in the treatment of cancer in a subject in need thereof.

The present invention thus relates to an isolated protein, an antibody or antigen-binding fragment thereof or a fusion protein as described hereinabove (preferably in a composition, pharmaceutical composition or medicament as describe hereinabove), for treating or for use in the treatment of cancer.

In one embodiment, a therapeutically effective amount of said protein, antibody or antigen-binding fragment thereof or fusion protein is administered or is to be administered to the subject.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In another embodiment, the isolated protein, antibody or antigen-binding fragment thereof or fusion protein according to the present invention may be used in the treatment of an infectious disease, disorder or symptom thereof in a subject in need thereof.

In one embodiment, a therapeutically effective amount of a protein, of an antibody or antigen-binding fragment thereof or of a fusion protein of the present invention is administered or is to be administered to the subject.

The present invention thus relates to an isolated protein, an antibody or antigen-binding fragment thereof or a fusion protein as described hereinabove (preferably in a composition, pharmaceutical composition or medicament as describe hereinabove), for treating or for use in the treatment of infectious disease, disorder or symptom.

As used herein the term “infectious disease” includes any infection caused by viruses, bacteria, protozoa, molds or fungi. In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses.

In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease. Bacterial infections that can be treated according to this invention include, but are not limited to, infections caused by the following: Staphylococcus; Streptococcus, including S. pyogenes; Enterococci; Bacillus, including Bacillus anthracis, and Lactobacillus; Listeria; Corynebacterium diphtheriae; Gardnerella including G. vaginalis; Nocardia; Streptomyces; Thermoactinomyces vulgaris; Treponerna; Camplyobacter, Pseudomonas including aeruginosa; Legionella; Neisseria including N. gonorrhoeae and N. meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachomatis; Mycobacterium including M. tuberculosis, M. intracellulare, M. folluiturn, M. laprae, M. avium, M. bovis, M. africanum, M. kansasii, M. intracellulare, and M. lepraernurium; and Nocardia. Protozoa infections that may be treated according to this invention include, but are not limited to, infections caused by leishmania, kokzidioa, and trypanosoma. A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the compositions according to the invention.

In one embodiment, the isolated protein, antibody or antigen-binding fragment thereof, fusion protein, nucleic acid, expression vector, composition, pharmaceutical composition or medicament according to the present invention is used alone.

In another embodiment, the isolated protein, antibody or antigen-binding fragment thereof, fusion protein, nucleic acid, expression vector, composition, pharmaceutical composition or medicament according to the present invention is used in combination with one further therapeutic agent, e.g. a chemotherapeutic agent, a targeted cancer therapy, radiotherapy or an immunotherapeutic agent, or an agent that may be used for treating an infectious disease.

Such administration may be simultaneous, separate or sequential. For simultaneous administration the agents may be administered as one composition or as separate compositions, as appropriate. The further therapeutic agent is typically relevant for disorders to be treated. Exemplary therapeutic agents include for example anti-cancer antibodies, cytotoxic agents, chemotherapeutic agents, anti-angiogenic agents, anti-cancer immunogens, cell cycle control/apoptosis regulating agents, hormonal regulating agents, and other immunosuppressive and/or anti-inflammatory drugs selected from corticoids, such as glucocorticoids.

In one embodiment, the isolated protein, antibody or antigen-binding fragment thereof, fusion protein, nucleic acid, expression vector, composition, pharmaceutical composition or medicament according to the present invention is used in combination with a chemotherapeutic agent.

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin;

nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids , e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In one embodiment, the isolated protein, antibody or antigen-binding fragment thereof, fusion protein, nucleic acid, expression vector, composition, pharmaceutical composition or medicament according to the present invention is used in combination with a targeted cancer therapy.

As used herein, the term “targeted cancer therapies” are drugs or other substances that block the growth and spread of cancer by interfering with specific molecules (“molecular targets”) that are involved in the growth, progression, and spread of cancer. Targeted cancer therapies are sometimes called “molecularly targeted drugs”, “molecularly targeted therapies”, “precision medicines”, or similar names In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; TI571), leflunomide (SU101), vandetanib (Zactima; ZD6474), MK-2206 (8-P-aminocyclobutyllphenyll -9-phenyl-1,2,4-triazolo [3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In one embodiment, the isolated protein, antibody or antigen-binding fragment thereof, fusion protein, nucleic acid, expression vector, composition, pharmaceutical composition or medicament according to the present invention is used in combination with radiotherapy.

The term “radiotherapy” may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In one embodiment, the isolated protein, antibody or antigen-binding fragment thereof, fusion protein, nucleic acid, expression vector, composition, pharmaceutical composition or medicament according to the present invention is used in combination with an immunotherapeutic agent or immunotherapy.

The terms “immunotherapeutic agent” or “immunotherapy” as used herein, refers to a compound, composition or treatment that indirectly or directly enhances, stimulates or increases the body's immune response against cancer cells and/or that decreases the side effects of other anticancer therapies Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents or immunotherapies known in the art include, but are not limited to: cytokines, checkpoint inhibitors, checkpoint agonists also referred to as T cell agonists, antibodies including monoclonal antibodies, antibody domains, antibody fragments, bispecific antibodies, preventive and therapeutic vaccines, oncolytic viruses, adoptive transfer of immune cells (T cells, NK, cells, dendritic cells, B cells . . . ).

One of the central premises underlying cancer immunotherapy is the presence of antigens which are selectively or abundantly expressed or mutated in cancer cells, thus enabling the specific recognition and subsequent destruction of the cancer cells. Such antigens are commonly referred to as tumor-specific antigens. Another of the central premises underlying cancer immunotherapy is the presence of lymphocytes in the tumors, i.e., tumor infiltrating lymphocytes (TILs), and notably of effector TILs which can target and kill the tumor cells through the recognition of the above-mentioned tumor-specific antigens.

Immunotherapeutic agents or therapies can be passive. A passive immunotherapeutic agent is one that produces an immediate action due to the administration of immune-cell factors, like monoclonal antibodies. The results of a passive immunotherapy are tied temporally to administration of the agent, therefore continued dosing may be required for a prolonged response). In another embodiment, the immunotherapeutic agent or therapies are active. An active immunotherapeutic agent is one that produces a lasting, durable response by way of inducing immunological memory. This most closely resembles a normal immune response. However, just as immune system function varies in a healthy population, the level of response to an active immunotherapy agent depends on individual factors).

Active immunotherapeutic agents include both non-specific active agents (i.e., agents that boost the immune system generally so that the human body becomes more effective in fighting the growth and/or spread of cancer cells), and specific active agents, (i.e., agents inducing the generation of cell-mediated and antibody immune responses focused on specific antigens expressed by the cancer cells). Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g., cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

In one embodiment, the isolated protein, antibody or antigen-binding fragment thereof, fusion protein, nucleic acid, expression vector, composition, pharmaceutical composition or medicament according to the present invention is used in combination with a cytokine therapy.

As used herein, a “cytokine therapy” is defined as the administration of at least one cytokine to the subject.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognize and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

In one embodiment, the isolated protein, antibody or antigen-binding fragment thereof, fusion protein, nucleic acid, expression vector, composition, pharmaceutical composition or medicament according to the present invention is used in combination with a cytokine mimetics, such as, for example, an IL-2 mimetics. In one embodiment, the IL-2 mimetics is not capable of binding CD25. In one embodiment, the IL-2 mimetics binds preferentially to an IL-2R comprising the 13 and y subunits as compared to an IL-2R comprising the α, β and γ subunits. A non-limitative example of IL-2 mimetics that may be used is NKTR-214 (Nektar Therapeutics).

In one embodiment, the isolated protein, antibody or antigen-binding fragment thereof, fusion protein, nucleic acid, expression vector, composition, pharmaceutical composition or medicament according to the present invention is used in combination with a checkpoint inhibitor therapy.

As used herein, a “checkpoint inhibitor therapy” is defined as the administration of at least one checkpoint inhibitor to the subject.

Checkpoint inhibitors (CPI, that may also be referred to as immune checkpoint inhibitors or ICI) molecules, often antibodies, block the interactions between inhibitory receptors (IRs) expressed on T cells and their ligands. As a cancer treatment, checkpoint inhibitor therapy aims at preventing the activation of inhibitory receptors expressed on T cells by ligands expressed by the tumor cells. Checkpoint inhibitor therapy thus aims at preventing the inhibition of T cells present in the tumor, i.e., tumor infiltrating T cells, and thus at enhancing the subject immune response towards the tumor cells.

Examples of checkpoint inhibitors include, without being limited to, inhibitors of the cell surface receptor PD-1 (programmed cell death protein 1), also known as CD279 (cluster differentiation 279); inhibitors of the ligand PD-L1 (programmed death-ligand 1), also known as CD274 (cluster of differentiation 274) or B7-H1 (B7 homolog 1); inhibitors of the cell surface receptor CTLA4 or CTLA-4 (cytotoxic T-lymphocyte-associated protein 4), also known as CD152 (cluster of differentiation 152); inhibitors of LAG-3 (lymphocyte-activation gene 3), also known as CD223 (cluster differentiation 223); inhibitors of TIM-3 (T-cell immunoglobulin and mucin-domain containing-3), also known as HAVCR2 (hepatitis A virus cellular receptor 2) or CD366 (cluster differentiation 366); inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3); inhibitors of BTLA (B and T lymphocyte attenuator), also known as CD272 (cluster differentiation 272); inhibitors of CEACAM-1 (carcinoembryonic antigen-related cell adhesion molecule 1) also known as CD66a (cluster differentiation 66a).

In one embodiment, the at least one checkpoint inhibitor is selected from the group comprising or consisting of inhibitors of PD-1, inhibitors of PD-L1, inhibitors of CTLA-4 and any mixtures thereof.

In one embodiment, the isolated protein, antibody or antigen-binding fragment thereof, fusion protein, nucleic acid, expression vector, composition, pharmaceutical composition or medicament according to the present invention is used in combination with a checkpoint agonist therapy.

As used herein, a “checkpoint agonist therapy” is defined as the administration of at least one T checkpoint agonist to the subject.

Checkpoint agonists act by activating stimulatory receptors (costimulatory receptors) expressed on immune cells, such as T cells. As used herein, the term “stimulatory receptors” refers to receptors that induce a stimulatory signal upon activation, and thus lead to an enhancement of the immune response. As a cancer treatment, checkpoint agonist therapy aims at activating stimulatory receptors expressed on immune cells present in a tumor. In particular, T-cell agonist therapy aims at enhancing the activation of T cells present in a tumor, i.e., tumor infiltrating T cells, and thus at enhancing the subject immune response towards the tumor cells. Currently, a number of potential targets for checkpoint agonist therapy have been identified.

Examples of checkpoint agonists include, without being limited to, agonists of CD137 (cluster differentiation 137) also known as 4-1BB or TNFRS9 (tumor necrosis factor receptor superfamily, member 9); agonists of OX40 receptor also known as CD134 (cluster differentiation 134) or TNFRSF4 (tumor necrosis factor receptor superfamily, member 4); agonists of GITR (glucocorticoid-induced TNF receptor family-related protein); agonists of ICOS (inducible co-stimulator); agonists of CD27-CD70 (cluster differentiation 27-cluster differentiation 70); and agonists of CD40 (cluster differentiation 40).

In one embodiment, the at least one checkpoint agonist is selected from the group comprising or consisting of agonists of CD137, agonists of OX40, agonists of GITR, agonists of ICOS, agonists of CD27-CD70, agonists of CD40 and any mixtures thereof.

In one embodiment, the isolated protein, antibody or antigen-binding fragment thereof, fusion protein, nucleic acid, expression vector, composition, pharmaceutical composition or medicament according to the present invention is used in combination with a second antibody that is specific for an immune receptor or a costimulatory molecule.

Examples of antibodies that are specific for an immune receptor include but are not limited to anti-CTLA4 antibodies (e.g. Ipilimumab), anti-PD1 antibodies, anti-PDL1 antibodies, anti-TIM3 antibodies, anti-LAG3 antibodies, anti-B7H3 antibodies, anti-B7H4 antibodies, anti-B7H6 antibodies, anti-4-1BB antibodies and anti-OX40 antibodies. Other examples of antibodies specific for an immune receptor are anti-TIGIT antibodies.

In another embodiment, the antibody or antigen-binding fragment thereof is bispecific, and is further capable of binding to an immune receptor or to a costimulatory molecule.

Examples of immune receptors include, but are not limited to, CTLA4, PD1, PDL1, TIM3, LAG3, B7H3, B7H4, B7H6, 4-1BB, TIGIT and OX40.

Examples of costimulatory molecules include, but are not limited to, CTLA4, PD1, PDL1, TIM3, LAG3, B7H3, B7H4, B7H6, 4-1BB and OX40.

In one embodiment, the isolated protein, antibody or antigen-binding fragment thereof, fusion protein, nucleic acid, expression vector, composition, pharmaceutical composition or medicament according to the present invention is used in combination with a second antibody that induces, via ADCC, the death of a cell expressing an antigen to which the second antibody binds. In one embodiment, the second antibody (e.g. of IgG1 or IgG3 isotype) induces ADCC toward a cell to which the antibody binds. NK cells have an important role in inducing ADCC and increased reactivity of NK cells can be directed to target cells through use of such a second antibody. In one embodiment, the second antibody is specific for a cell surface antigen, e.g., membrane antigen. In some embodiments, the second antibody is specific for a tumor antigen as described above (e.g., molecules specifically expressed by tumor cells), such as CD20, CD52, ErbB2 (or HER2/Neu), CD33, CD22, CD25, MUC-1, CEA, KDR, ocV133, etc., particularly lymphoma antigens (e.g., CD20). Accordingly, the present invention also provides methods to enhance the anti-tumor effect of monoclonal antibodies directed against tumor antigen(s).

In the methods of the invention, ADCC function is specifically augmented, which in turn enhances target cell killing, by sequential administration of an antibody directed against one or more tumor antigens, and an antibody of the present invention.

In one embodiment, the isolated protein, antibody or antigen-binding fragment thereof, fusion protein, nucleic acid, expression vector, composition, pharmaceutical composition or medicament according to the present invention is used in combination with a natural ligand of an NK cell activating receptor or an antibody that binds and activates an NK cell activating receptor.

In one embodiment, the agent is an agent that increases the presence of a natural ligand of an NK cell activating receptor on the surface of a target cell (e.g., infected cells, or tumor cells). As used herein, the term “activating NK receptor” refers to any molecule on the surface of NK cells that, when stimulated, causes a measurable increase in any property or activity known in the art as associated with NK activity, such as cytokine (for example IFN-γ and TNF-α) production, increases in intracellular free calcium levels, the ability to target cells in a redirected killing assay, or the ability to stimulate NK cell proliferation. Examples of “activating NK receptors” include but are not limited to activating forms of KIR proteins (for example KIR2DS proteins), CD160-TM, NKG2D, IL-2R, IL-12R, IL-15R, IL-18R and IL-21R. Examples of ligands that act as agonists at activating receptors include, e.g. IL-2, IL-15, IL-21 polypeptides. In some embodiments, the second antibody is specific for CD137. As used herein the term “CD137” has its general meaning in the art and may also be referred to as Ly63, ILA or 4-1BB. CD137 is a member of the tumor necrosis factor (TNF) receptor family Members of this receptor family and their structurally related ligands are important regulators of a wide variety of physiologic processes and play an important role in the regulation of immune responses.

CD137 is expressed by activated NK cells, T and B lymphocytes and monocytes/macrophages. The gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The ligand for the receptor is TNFSF9. Human CD137 is reported to bind only to its ligand. Agonists include the native ligand (TNFSF9), aptamers (see McNamara et al. (2008) J. Clin. Invest. 1 18: 376-386), and antibodies.

In one embodiment, the isolated protein, antibody or antigen-binding fragment thereof, fusion protein, nucleic acid, expression vector, composition, pharmaceutical composition or medicament according to the present invention is used in combination with a therapeutic vaccine or treatment vaccine.

As used herein, a therapeutic vaccine is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells; and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; RNA-based vaccines, and dendritic cell vaccines.

In one embodiment, the isolated protein, antibody or antigen-binding fragment thereof, fusion protein, nucleic acid, expression vector, composition, pharmaceutical composition or medicament according to the present invention is used in combination with an oncolytic virus therapy.

As used herein, an oncolytic virus therapy is defined as the administration of at least one oncolytic virus to the subject.

Oncolytic viruses are defined as viruses that preferentially infect and kill cancer cells over normal, non-cancer, cells. As a cancer treatment, oncolytic virus therapy aims at killing cancer cells and/or triggering or enhancing an immune response towards the cancer cells.

Examples of oncolytic viruses include, without being limited to, modified herpes simplex type-1 viruses such as talimogene laherparepvec (or T-VEC) or HSV-1716; modified adenoviruses such as Ad5-DNX-2401; modified measles viruses such as MV-NIS; modified vaccinia viruses (VV) such as vaccinia virus TG6002; and modified polioviruses such as PVS-RIPO.

In one embodiment, the isolated protein, antibody or antigen-binding fragment thereof, fusion protein, nucleic acid, expression vector, composition, pharmaceutical composition or medicament according to the present invention is used in combination with an adoptive transfer of cells, also referred to as adoptive cell therapy (both also referred to as ACT), such as, for example, an adoptive transfer of T cells or NK cells, also referred to as adoptive T cell therapy or adoptive NK cell therapy, respectively.

As used herein, an “adoptive transfer of cells” or “adoptive cell therapy” is defined as the transfer, for example as an infusion or re-infusion, of immune cells to a subject. As a cancer treatment, the adoptive transfer of immune cells to a subject aims at enhancing the subject immune response towards the cancer cells.

Examples of immune cells that may be used for a cell therapy include without limitation cytotoxic cells (e.g., natural killer (NK) cells, CD8⁺ T cells, and natural killer (NK) cells T cells), effector T cells (e.g., CD4⁺ T cells and CD8⁺ T cells), alpha beta (αβ) T cells, and gamma delta (γδ) T cells, antibody-expressing B cells or other antibody-producing or -presenting cells and dendritic cells.

In one embodiment, the transferred immune cells as described hereinabove are antigen-specific cells. In one embodiment, the transferred immune cells as described hereinabove are antigen-specific immune cells, wherein said antigen is specifically and/or abundantly expressed by cancer cells. In one embodiment, the transferred immune cells as described hereinabove are tumor-specific immune cells, in other words the transferred immune cells as described hereinabove specifically recognize cancer cells or tumor cells through an antigen specifically and/or abundantly expressed by said cancer cells or tumor cells. In one embodiment, the transferred immune cells as described hereinabove are tumor-specific effector T cells. In one embodiment, the transferred immune cells as described hereinabove are tumor-specific CD8⁺ effector T cells, in particular tumor-specific cytotoxic CD8⁺ T cells. In one embodiment, the transferred cells are tumor infiltrating cell (TIL). In one embodiment, the transferred immune cells as described hereinabove are tumor-specific cytotoxic cells. In one embodiment, the transferred immune cells as described hereinabove are tumor-specific NK cells.

Examples of tumor-specific antigens, i.e.., antigens that are specifically and/or abundantly expressed by cancer cells include, without being limited to, neoantigens (also referred to as new antigens or mutated antigens), 9D7, ART4, β-catenin, BING-4, Bcr-abl, BRCA1/2, calcium-activated chloride channel 2, CDK4, CEA (carcinoembryonic antigen), CML66, Cyclin B1, CypB, EBV (Epstein-Barr virus) associated antigens (such as LMP-1, LMP-2, EBNA1 and BARF1), Ep-CAM, EphA3, fibronectin, Gp100/pme117, Her2/neu, HPV (human papillomavirus) E6, HPV E7, hTERT, IDH1, IDH2, immature laminin receptor, MC1R, Melan-A/MART-1, MART-2, mesothelin, MUC1, MUC2, MUM-1, MUM-2, MUM-3, NY-ESO-1/LAGE-2, p53, PRAME, prostate-specific antigen (PSA), PSMA (prostate-specific membrane antigen), Ras, SAP-1, SART-I, SART-2, SART-3, SSX-2, survivin, TAG-72, telomerase, TGF-βRII, TRP-1/-2, tyrosinase, WT1, antigens of the BAGE family, antigens of the CAGE family, antigens of the GAGE family, antigens of the MAGE family, antigens of the SAGE family, and antigens of the XAGE family.

As used herein, neoantigens (also referred to as new antigens or mutated antigens) correspond to antigens derived from proteins that are affected by somatic mutations or gene rearrangements acquired by the tumors. Neoantigens may be specific to each individual subject and thus provide targets for developing personalized immunotherapies. Examples of neoantigens include for example, without being limited to, the R24C mutant of CDK4, the R24L mutant of CDK4, KRAS mutated at codon 12, mutated p53, the V599E mutant of BRAF and the R132H mutant of IDH1.

In one embodiment, the transferred immune cells as described hereinabove are specific for a tumor antigen selected from the group comprising or consisting of the class of CTAs (cancer/testis antigens, also known as MAGE-type antigens), the class of neoantigens and the class of viral antigens.

As used herein, the class of CTAs corresponds to antigens encoded by genes that are expressed in tumor cells but not in normal tissues except in male germline cells. Examples of CTAs include, without being limited to, MAGE-AL MAGE-A3, MAGE-A4, MAGE-C2, NY-ESO-1, PRAME and SSX-2.

As used herein, the class of viral antigens corresponds to antigens derived from viral oncogenic proteins. Examples of viral antigens include, without being limited to, HPV (human papillomavirus) associated antigens such as E6 and E7, and EBV (Epstein-Barr virus) associated antigens such as LMP-1, LMP-2, EBNA1 and BARF1.

In one embodiment, the transferred immune cells as described hereinabove are autologous immune cells, in particular autologous T cells. In another embodiment, the transferred immune cells as described hereinabove are allogenic (or allogenous) immune cells, in particular allogenic NK cells.

For example, autologous T cells can be generated ex vivo either by expansion of antigen-specific T cells isolated from the subject or by redirection of T cells of the subject through genetic engineering.

In one embodiment, the immune cells to be infused are modified ex vivo before being infused to the subject.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In one embodiment, the isolated protein, antibody or antigen-binding fragment thereof, fusion protein, nucleic acid, expression vector, composition, pharmaceutical composition or medicament according to the present invention is used in combination with a CAR immune cell therapy, in particular a CAR T cell therapy or a CAR NK cell therapy.

As used herein, CAR immune cell therapy is an adoptive cell therapy wherein the transferred cells are immune cells as described hereinabove, such as T cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of cancer cells. In one embodiment, the transferred CAR T cells recognize a tumor-specific antigen.

In another embodiment, the transferred NK cells as described hereinabove are CAR NK cells. The expression of a CAR allows the NK cells to be redirected against a selected antigen, such as an antigen expressed at the surface of cancer cells. In one embodiment, the transferred CAR NK cells recognize a tumor-specific antigen.

Examples of tumor-specific antigens are mentioned hereinabove.

In one embodiment, the transferred CAR T cells or CAR NK cells recognize a tumor-specific antigen selected from the group comprising or consisting of EGFR and in particular EGFRvIII, mesothelin, PSMA, PSA, CD47, CD70, CD133, CD171, CEA, FAP, GD2, HER2, IL-13Rα, αvβ6 integrin, ROR1, MUC1, GPC3, EphA2, CD19, CD21, and CD20.

In one embodiment, the CAR immune cells as described hereinabove are autologous CAR immune cells, in particular autologous CAR T cells. In another embodiment, the CAR immune cells as described hereinabove are allogenic (or allogenous) CAR immune cells, in particular allogenic CAR NK cells.

In one embodiment, the isolated protein, antibody or antigen-binding fragment thereof, fusion protein, nucleic acid, expression vector, composition, pharmaceutical composition or medicament according to the present invention is used in combination with an antibiotic. Examples of antibiotics include, but are not limited to, penicillins (e.g., penicillin, amoxicillin), tetracyclines (e.g., doxycyclient, tetracycline, minocycline), cephalosporins (e.g., cefuroxime, ceftriaxone, cefdinir), quinolones (e.g., ciprofloxacin, levofloxacin, moxifloxacin), lincomycins (e.g., clindamycin, lincomycin), macrolides (e.g., azithromycin, clarithromycin, erythromycin), sulfonamides (e.g., sulfamethoxazole-trimethoprim, sulfasalazine, sulfisoxazole), glycopeptides (e.g., dalbavancin, oritavancin, telavancin, vancomycin), aminoglycosides (e.g., gentamicin, tobramycin, amikacin) and carbapenems (e.g., imipenem, meropenem, doripenem, ertapenem).

In one embodiment, the isolated protein, antibody or antigen-binding fragment thereof, fusion protein, nucleic acid, expression vector, composition, pharmaceutical composition or medicament according to the present invention is used in combination with an antiviral drug. Examples of antiviral drugs include, but are not limited to, abacavir, acyclovir, adefovir, amantadine, amprenavir, ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever famciclovir, fomivirsen, fosamprenavir, foscarnet, fosfonet, ibacitabine, imunovir, idoxuridine, imiquimod, indinavir, inosine, integrase inhibitor, interferon type III, interferon type II, interferon type I, interferon, lamivudine, lopinavir, loviride, maraviroc, moroxydine, methisazone, nelfinavir, nevirapine, nexavir, nitazoxanide, nucleoside analogues, norvir, oseltamivir, peginterferon alfa-2a, penciclovir, peramivir, pleconaril, podophyllotoxin, protease inhibitor, raltegravir, reverse transcriptase inhibitor, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

In one embodiment, the isolated protein, antibody or antigen-binding fragment thereof, fusion protein, nucleic acid, expression vector, composition, pharmaceutical composition or medicament according to the present invention is used in combination with an antifungal agent. Examples of antifungal agents include, but are not limited to, polyene antifungals (e.g., amphotericin B, candicidin, filipin, hamycin, natamycin, nystatin, rimocidin), imidazole antifungals (e.g., bifonazole, butoconazole, clotrimazole, econazole, fenticonazole, isoconazole, ketoconazole, luliconazole, miconazole, omoconazole, oxiconazole, sertaconazole, sulconazole, tioconazole), triazole antifungals (e.g., albaconazole, efinaconazole, epoxiconazole, fluconazole, isavuconazole, itraconazole, posaconazole, propiconazole, ravuconazole, terconazole, voriconazole), thiazole antifungals (e.g., abafungin), allylamines, echinocandins (e.g., anidulafungin, caspofungin, micafungin).

In one embodiment, the isolated protein, antibody or antigen-binding fragment thereof, fusion protein, nucleic acid, expression vector, composition, pharmaceutical composition or medicament according to the present invention is used in combination with an anti-parasitic agent. Examples of anti-parasitic agents include, but are not limited to, broad-spectrum anti-parasitic agents (e.g., nitazoxanide), antiprotozoals (e.g., melarsoprol, eflornithine, metronidazole, tinidazole, miltefosine), antihelminthic (including, without limitation, antinematodes (ancylostoma caninum, mebendazole, pyrantel pamoate, thiabendazole, diethylcarbamazine, ivermectin), anticestodes (e.g., niclosamide, praziquantel, albendazole), antitrematodes (e.g., praziquantel)), antiamoebics (e.g., rifampin, amphotericin B).

Another object of the present invention relates to the use of the antibody or antigen-binding fragment thereof with another therapeutic agent as described hereinabove, in the treatment of diseases in a subject in need thereof, wherein said antibody or antigen-binding fragment thereof is used as an adjuvant for the therapeutic agent.

Another object of the present invention relates to the use of the fusion protein as described herein with another therapeutic agent as described hereinabove, in the treatment of diseases in a subject in need thereof, wherein said fusion protein is used as an adjuvant for the therapeutic agent.

The present invention thus relates to an antibody or antigen-binding fragment thereof as described herein or to a fusion protein as described herein (preferably in a composition, pharmaceutical composition or medicament), for use as an adjuvant in a cancer therapy. The present invention thus relates to an antibody or antigen-binding fragment thereof or a fusion protein as described herein (preferably in a composition, pharmaceutical composition or medicament), for use as an adjuvant in a therapy for an infectious disease.

In one embodiment, the present invention relates to the use of the antibody or fragment thereof as described herein or of the fusion protein as described herein, for potentiating an immune response induced by a cancer therapy in a patient in need thereof.

In one embodiment, the antibody or fragment thereof according to the present invention or the fusion protein according to the present invention may be used as immunotherapeutic agent, particularly to treat a wide variety of cancers (e.g., cancers associated with immunosuppression and/or immune exhaustion).

In one embodiment, the antibody or fragment thereof or the fusion protein according to the present invention may potentiate an immune response induced by a cancer therapy in a patient comprising administering said antibody or fragment thereof or said fusion protein to a subject in an amount effective to potentiate an immune response induced by the cancer therapy in the patient.

As used herein, the term “adjuvant” refers to a compound or a combination of compounds that potentiates at therapy, such as, for example, a cancer therapy. Adjuvants may increase the effective immune response against low or non-immunogenic tumor cells. In one embodiment, the adjuvant is used with a well-known cancer therapeutic agent in the treatment of cancer and thus potentiates the immune response towards cancer cells. For example, an adjuvant may potentiate an immune response during a cancer therapy, decrease T cell exhaustion (without decreasing T cells activation), increase the survival of T cells, enhance NK cells cytotoxicity, decrease the tumor growth and/or the tumor size, and/or increase in survival, treats or prevents cancer metastasis. In one embodiment, potentiation of a cancer therapy in the presence of an adjuvant, is defined by comparison with a cancer therapy administered alone.

In another embodiment, the antibody or fragment thereof or the fusion protein as described herein can increase or improve the immune response of a subject.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments of any of the aspects, the response is specific for a particular antigen (an “antigen-specific response”), and refers to a response by a CD4 T cell, CD8 T cell, or B cell via their antigen-specific receptor. In some embodiments of any of the aspects, an immune response is a T cell response, such as a CD4⁺ response or a CD8⁺ response. Such responses by these cells can include, for example, cytotoxicity, proliferation, cytokine or chemokine production, trafficking, or phagocytosis, and can be dependent on the nature of the immune cell undergoing the response.

As with other known immunotherapeutic agents, the ability of the antibody or fragment thereof or of the fusion protein according to the present invention, to potentiate an immune response in a patient may have broader therapeutic implications outside the cancer field. For example, it has been proposed that immune potentiating agents may be useful in treating a wide variety of infectious diseases, particularly pathogenic agents which promote immunosuppression and/or immune exhaustion. Also, such immune potentiating agents may be useful in boosting the immunization efficacy of vaccines (e.g., infectious disease and cancer vaccines).

Another object of the present invention relates to the use of the antibody or antigen-binding fragment thereof according to the present invention, or of a fusion protein according to the present invention, to deplete CD25 expressing Treg cells in a subject in need thereof, wherein a therapeutically effective amount of an antibody or fragment thereof or of a fusion protein of the present invention is to be administered to the subject.

The present invention thus further relates to a method for depleting CD25 expressing Treg cells in a subject in need thereof, comprising administering to the subject an antibody or antigen-binding fragment thereof, a fusion protein, a composition, a pharmaceutical composition, or a medicament as described herein.

In one embodiment, the antibody or antigen-binding fragment thereof or the fusion protein as described hereinabove (preferably in a composition, pharmaceutical composition or medicament as describe hereinabove), is for use to deplete CD25 expressing Treg cells.

In one embodiment, the CD25 expressing Treg cells are tumor infiltrating Tregs.

In one embodiment, the antibody or antigen-binding fragment thereof as described hereinabove for use to deplete CD25 expressing Treg cells is an IgG, preferably an IgG1.

In one embodiment, the antibody or antigen-binding fragment thereof as described hereinabove for use to deplete CD25 expressing Treg cells binds to at least one activating Fcγ Receptor, preferably selected from FcγRI, FcγRIIa, FcγRIII with a high affinity.

In one embodiment, the antibody or antigen-binding fragment thereof as described hereinabove for use to deplete CD25 expressing Treg cells elicits an enhanced ADCC, ADCP and/or CDC response, preferably an increased ADCC and/or ADCP response, more preferably an increased ADCC response.

In one embodiment, the antibody or antigen-binding fragment thereof as described for use to deplete CD25 expressing Treg cells does not inhibit the IL-2 signaling via CD25. Thus, in one embodiment, the antibody or antigen-binding fragment thereof as described for use to deplete CD25 expressing Treg cells does not inhibit the proliferation and/or activation of CD4⁺ and CD8⁺ T cells (or effector T cells). In another embodiment, the antibody or antigen-binding fragment thereof as described for use to deplete CD25 expressing Treg cells does not inhibit the phosphorylation of STATSa in CD4⁺ and CD8⁺ T cells (or effector T cells).

As compared to the anti-CD25 antibodies of the prior art, the antibodies of the present invention may present the following advantages:
in some embodiments, the antibody of the present invention presents an increased affinity for CD25, as compared to the CD25 antibodies of the prior art;
in some embodiments, the antibody of the present invention presents an increased avidity for CD25, as compared to the CD25 antibodies of the prior art;
in some embodiments, the antibody of the present invention induces an increased IL-2-dependent activation of T cells in culture, and preferably an increased proliferation of T cells in culture;
in some embodiment, the antibody of the present invention induces a lower inhibition of IL-2-induced T cell proliferation in culture as compared to the CD25 antibodies of the prior art;
in some embodiments, the antibody of the present invention presents an increased ADCC activity on CD25⁺ expressing cells, preferably CD25⁺ expressing T cells and more preferably CD25⁺ expressing Treg cells as compared to the CD25 antibodies of the prior art;
in some embodiments, the antibody of the present invention presents an increased ADCP activity on CD25⁺ expressing cells, preferably CD25⁺ expressing T cells and more preferably CD25⁺ expressing Treg cells as compared to the CD25 antibodies of the prior art;
in some embodiments, the antibody of the present invention induces depletion (preferably in vivo depletion) of CD25⁺ expressing cells, preferably CD25⁺ expressing T cells and more preferably CD25⁺ expressing Treg cells, with a higher efficiency as compared to the CD25 antibodies of the prior art.

### EXAMPLES

Material and Methods

Binding of CD25 Specific mAbs:

The CD25 positive SU-DHL1 cell line was incubated with either FITC-labeled mIgG control or ALD25-H1, H2 and H4 anti-CD25 antibodies at 10 μg/ml for 30 min at 4° C. Cells were washed with PBS before flow cytometry analysis (LSR fortessa).

IL2 Binding Assay

SU-DHL1 cells were pre-incubated with human IgG1 control isotype, ALD25H4 or Basiliximab (1 or 10 μg/ml) for 1 h at room temperature. Cells were then sequentially incubated for 30 min at room temperature with biotinylated-conjugated IL2 (125 ng/ml) and APC-coupled streptavidin. A PBS wash was performed between each step of the procedure. Cells were analysed by flow cytometry on a Cytoflex (Beckman Coulter) and data analysed with FlowJo software. Data are expressed as the mean±SD of the mean fluorescence intensity of APC on gated live SU-DHL1 cells (n=3).

Binding Competition with “IL2 Non-Competitive” mAb 7G7B6:

The CD25 positive SU-DHL1 cell line was pre-incubated with either commercially available control huIgG or recombinant purified huALD25-H1, H2 or H4 antibodies at 10 μg/ml for 30 min at 4° C. Cells were washed with PBS and stained with 7G7B6-APC antibody (10 microg/ml) or APC-IgG control (Control mAb histogram) for 30 min at 4° C. Cells were then washed with PBS before analysis.

Impact on IL-2 Induced T Cell Proliferation:

Freshly isolated peripheral mononuclear cells (PBMC) were cultured in RPMI medium (10% FCS, 2% glutamin, 1% antibiotics) completed with PHA at 5 μg/ml for 72 h. PBMC were stained with CFSE, starved 24 h and activated T cells were then isolated by magnetic cell sorting. T cells were cultured 72 h with IL-2 at 50 UI/ml and antibodies at 1 μg/ml. Cell division was followed by flow cytometry.

CD25 Positive Cells Specific Lysis by Antibody Dependent Cell Cytotoxicity (ADCC):

Anti-CD25 induced antibody dependent cell cytotoxicity (ADCC) was obtained by coculturing IL-2 pre-activated PBMC (as effector cells) and CFSE-stained SU-DHL1 (target cells) at different ratios and with either hIgG control or anti-CD25 antibodies at 1 μg/ml or 10 μg/mL for 16 h. Cell specific lysis was obtained by colorimetric method, quantifying LDH release in the culture medium. Alternatively, cell death was evaluated by Flow Cytometry with a viability dye staining (7AAD). Percentage of CFSE+7AAD+ cells correspond to the target cells apoptosis.

CD25 Positive Cells Specific Lysis by Antibody Dependent Cell Phagocytosis (ADCP):

Anti-CD25 induced ADCP was obtained by coculturing THP-1 cells (as effector cells) and CFSE-stained SUDHL-1 cells (target cells) at different ratios and either hIgG control or anti-CD25 antibodies at 1 μg/ml for 4 h. At this time point, anti-CD33 APC antibody is added to the coculture. Cells are washed before Flow Cytometry analysis. Percentage of CD33+CFSE+ cells correspond to the level of induced phagocytosis.

IL-2 Induced T Cell Proliferation:

Freshly isolated peripheral mononuclear cells (PBMC) were cultured in RPMI medium (10% FCS, 2% glutamin, 1% antibiotics) completed with PHA at 5 μg/ml for 72 h. PBMC were stained with CFSE, and starved 24 h. Activated T cells were then isolated by magnetic cell sorting. Sorted T cells were cultured 72 h with IL-2 at 50 UI/ml and isotype controls or CD25-specific antibodies at lug/ml. Cell division was followed by flow cytometry by looking at CFSE dilution.

Impact on Treg Cells Depletion

CD34⁺ reconstituted humanized mice were injected with tumour cells. When tumours reached 100 mm³, mice were administered intraperitoneally with vehicle, ALD25H4 or 7G7B6 CD25-specific monoclonal antibodies at 10 mg/mL once a week. At day 29 after tumour engraftment, mice were sacrificed and the amount of CD4⁺CD25⁺FoxP3⁺ Treg cells were evaluated by Flow Cytometry.

Treg Depletion

PBMC (2×10⁶/ml) were incubated with human IgG1 control isotype, ALD25H1, ALD25H2, ALD25H4 or Basiliximab (10 μg/ml) plus anti-CD3/anti-CD28-coupled beads (Dako). After 6 days of incubation, labelings were performed using a mix of anti-CD8-FITC, -CD4-PE, CD39-PerCP-Cy5.5, -CD127-PE-Cy7, -CD3-APC and CD45-Pacific Blue antibodies. Cells were analysed by flow cytometry on a Cytoflex (Beckman Coulter) and data analysed with FlowJo software. Tregs were identified as the CD3⁺CD4⁺CD39⁺CD127^{low/-} cell population. Results are expressed as the mean %±SD of Tregs within the CD45+ lymphocyte population (n=3 donors).

Results

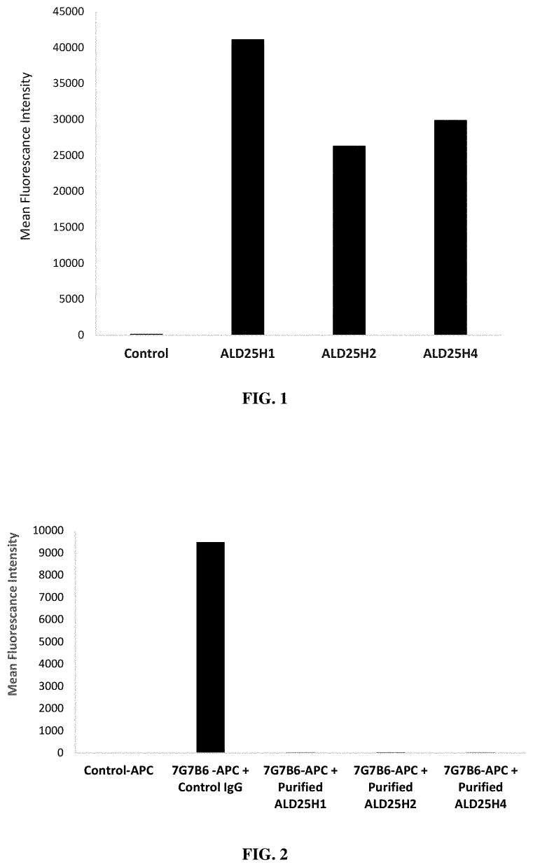

FIG. 1 shows that the antibodies of the present invention (ALD25H1, ALD25H2 and ALD25H4) are capable of binding to CD25. In addition, antibodies of the present invention showed negligible or low levels of background binding to cells non expressing CD25 (data not shown), demonstrating high specificity of the antibodies of the present invention for CD25.

FIG. 2 is the result of a competition assay, and demonstrate that the antibodies of the present invention compete with the monoclonal antibody 7G7B6. 7G7B6 is well known in the art to be an IL-2 non-competitive antibody.

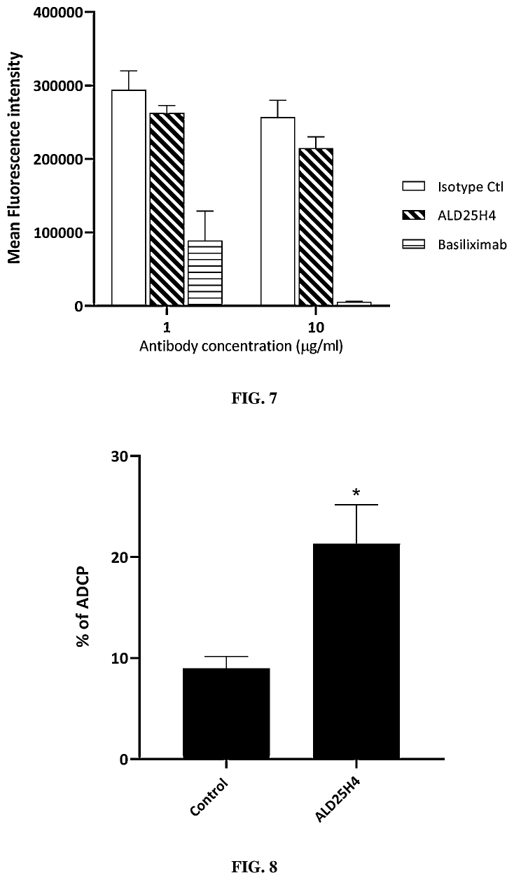

Moreover, FIG. 7 shows that the antibody of the present invention ALD25H4 does not significantly block the IL-2 binding on SUDHL-1 cells at 10 μg/mL whereas Basiliximab, an IL-2 blocking CD25-specific mAb, completely blocks IL-2 binding at the same concentration.

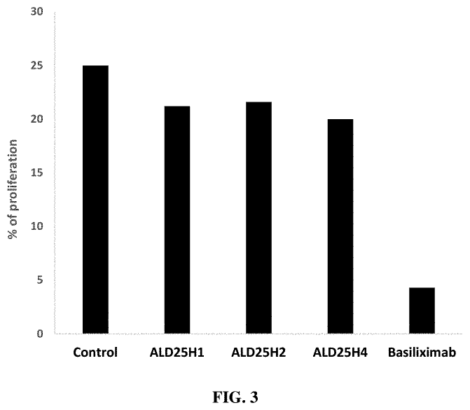

In addition, FIG. 3 shows that antibodies ALD25H1, ALD25H2 and ALD25H4 has no or very limited impact on IL-2 induced effector T cell proliferation.

Next, the ability of the antibodies of the present invention (ALD25H1, ALD25H2 and ALD25H4) to impact IL-2 induced T-cell proliferation was measured and compared to that of 7G7B6 and MA-251. For this purpose, PHA-activated T cells were isolated and cultured with 50 UI/m1 of IL-2 in order to induce a strong proliferation. Cells were grown in the presence of either isotype control or CD25-specific antibodies. As expected, and as shown in FIG. 5 and Table 1, Basiliximab inhibited the T cell proliferation by 90%.

In contrast ALD25H1, ALD25H2 and ALD25H4 only impacted the T cell proliferation induced by IL-2 between 20% to 30%. 7G7B6 and MA-251 showed significantly higher impact on IL-2 induced T cell growth with a constant 45% and 52% of inhibition respectively.

TABLE 1

| mAb | Control | ALD25H1 | ALD25H2 | ALD25H4 | 7G7B6 | MA-251 | Basiliximab |
|---|---|---|---|---|---|---|---|
| Mean | 100 | 78.1 | 81.7 | 73 | 55 | 47.6 | 10 |
| Sem | 0 | 4.1 | 3.2 | 9 | 1.8 | 0.3 | 3.5 |

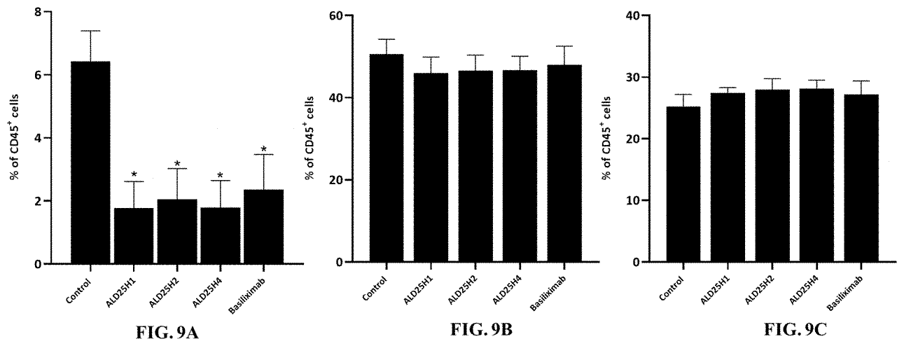

Then, the ability of the antibodies of the present invention to induce in vitro depletion of Tregs cells from a CD45⁺ lymphocytes population was measured and compared to that of basixilimab (FIG. 9). As shown in FIG. 9A, the antibodies of the present invention induce Treg cell depletion. This effect is specific to Treg cells, as no cell depletion was observed for CD4⁺ effector T cells nor for CD8⁺ effector T cells (FIGS. 9B and 9C).

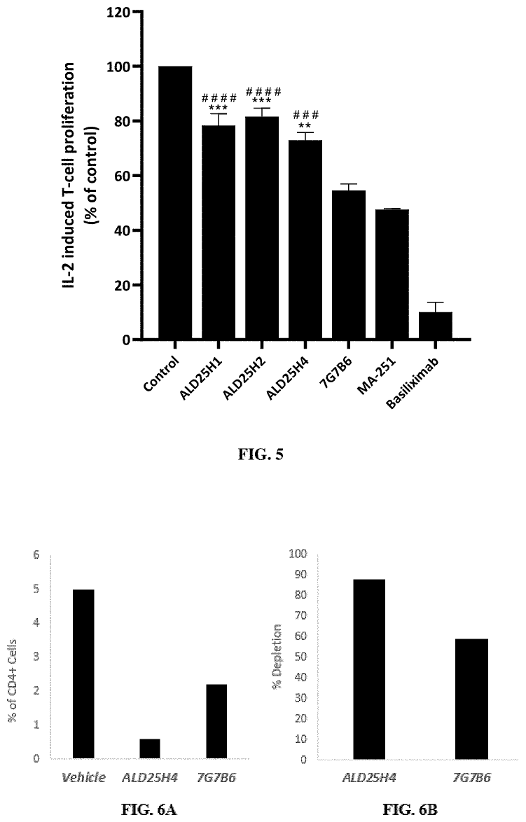

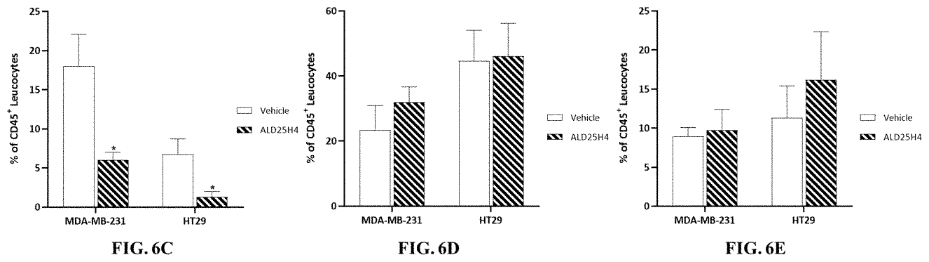

In addition, the ability of the antibody of the present invention (ALD25H4) to induce depletion of Treg cells was measured and compared to that of 7G7B6 in a mice model, where animals are injected with tumor cells. As shown in FIGS. 6A and B, the administration of ALD25H4 induced a massive depletion of CD4⁺CD25⁺FoxP3⁺ Treg cells within tumors, reaching 88% of depletion. By contrast, 7G7B6 was less effective to induce Treg cell depletion, as only 59% of Treg cells were depleted following administration of 7G7B6. In addition, as shown in FIGS. 6C, 6D and 6E, this depletion of T cells is specific of Treg cells, as no depletion of T effector cells, either TIL CD4⁺ T cells or TIL CD8⁺ T cells, is observed.

Taken together, these results demonstrate that the antibodies of the present invention are non-blocking antibodies, i.e., that they do not inhibit IL-2 signaling in T cells.

These results further demonstrate that ALD25H1, ALD25H2 and ALD25H4 binding to CD25 behave differently than 7G7B6 and MA-251, with significantly less impact on IL-2 activity. IL-2 being critical for T-cell proliferation and survival in cancer, the higher neutrality of ALD25H1, ALD25H2 and ALD25H4 for IL-2 function over 7G7B6 and MA-251 gives to these antibodies a critical advantage for the induction of cancer specific T-cell based immune responses.

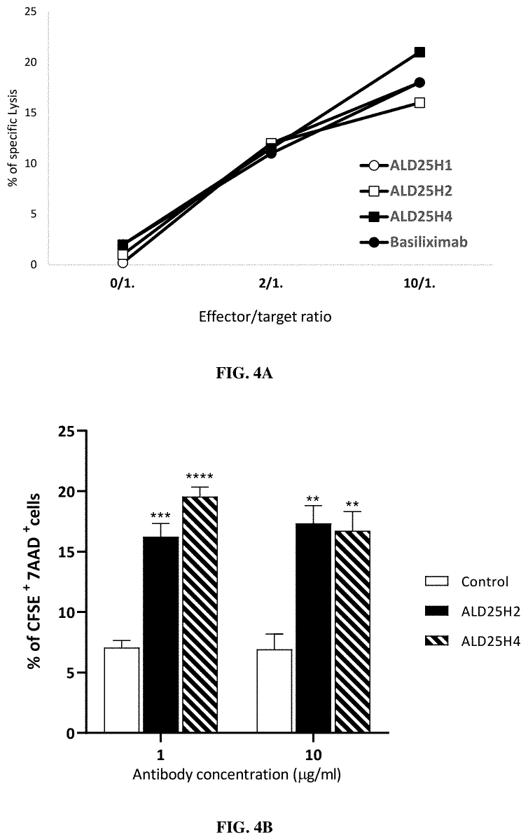

FIGS. 4A and 4B demonstrate that the antibodies of the present invention induce efficient specific lysis of CD25 positive cells by ADCC, with a percent of specific lysis equivalent to the one measured for basiliximab Moreover, the antibodies of the present invention also induce cell lysis by ADCP, as shown in FIG. 8.

Altogether, these results demonstrate the therapeutic potential of the antibodies of the present invention for treating cancer.

1-19. (canceled)

20. An isolated anti-human CD25 antibody or antigen-binding fragment thereof, wherein said antibody or fragment thereof does not inhibit the binding of interleukin-2 (IL-2) to CD25.

21. The isolated antibody or antigen-binding fragment thereof according to claim 20, wherein said antibody or fragment thereof is monoclonal.

22. The isolated antibody or antigen-binding fragment thereof according to claim 20, which is chimeric, humanized or human.

23. The isolated antibody or antigen-binding fragment thereof according to claim 20 comprising a heavy chain and a light chain,
wherein the variable region of the heavy chain (HCVR) comprises at least one of the three following complementary-determining regions (CDRs):
CDR1: X₄HAMA (SEQ ID NO: 1), wherein X₄ is D or N:
CDR2: YISYDGDNTYYRDSVKG (SEQ ID NO: 2); and
CDR3: GGNSGYD (SEQ ID NO: 3);
or any CDR having an amino acid sequence that shares at least about 70% of identity with SEQ ID NO: 1-3; and/or
wherein the variable region of the light chain (LCVR) comprises at least one of the three following CDRs:
CDR1: KX₁SQNVNKFX₂N (SEQ ID NO: 4), wherein X₁ is
A or G and wherein
X₂ is L or V;
CDR2: GTNSLQT (SEQ ID NO: 5);
and
CDR3: QQYX₃SWPWT (SEQ ID NO: 6),
wherein X₃ is S or T;
or any CDR having an amino acid sequence that shares at least about 70% of identity with SEQ ID NO: 4-6.

24. The isolated antibody or antigen-binding fragment thereof according to claim 23, wherein
(i) the HCVR comprises at least one of the three CDRs as defined in claim 4, and
(ii) the LCVR comprises at least one of the three CDRs as defined in claim 4.

25. The isolated antibody or antigen-binding fragment thereof according to claim 23, wherein:
the HCVR comprises the following CDRs:
CDR1: X₄HAMA (SEQ ID NO: 1), wherein X₄ is D or N;
CDR2: YISYDGDNTYYRDSVKG (SEQ ID NO: 2);
and
CDR3: GGNSGYD (SEQ ID NO: 3);
and
the LCVR comprises the following CDRs:
CDR1: KX₁SQNVNKFX₂N (SEQ ID NO: 4), wherein X₁ is
A or G and wherein X₂ is L or V;
CDR2: GTNSLQT (SEQ ID NO: 5);
and
CDR3: QQYX₃SWPWT (SEQ ID NO: 6),
wherein X₃ is S or T;
or any CDR having an amino acid sequence that shares at least about 70% of identity with said SEQ ID NO: 1-6.

26. The isolated antibody or antigen-binding fragment thereof according to claim 25, wherein:
the HCVR comprises the following CDRs:
CDR1: DHAMA (SEQ ID NO: 7);
CDR2: YISYDGDNTYYRDSVKG (SEQ ID NO: 2);
and
CDR3: GGNSGYD (SEQ ID NO: 3);
and
the LCVR comprises the following CDRs:
CDR1: KASQNVNKFLN (SEQ ID NO: 8);
CDR2: GTNSLQT (SEQ ID NO: 5);
and
CDR3: QQYSSWPWT (SEQ ID NO: 9).

27. The isolated antibody or antigen-binding fragment thereof according to claim 25, wherein:
the HCVR comprises the following CDRs:
CDR1: NHAMA (SEQ ID NO: 10);
CDR2: YISYDGDNTYYRDSVKG (SEQ ID NO: 2);
and
CDR3: GGNSGYD (SEQ ID NO: 3);
and
the LCVR comprises the following CDRs:
CDR1: KASQNVNKFVN (SEQ ID NO: 11);
CDR2: GTNSLQT (SEQ ID NO: 5);
and
CDR3: QQYSSWPWT (SEQ ID NO: 9).

28. The isolated antibody or antigen-binding fragment thereof according to claim 25, wherein:
the HCVR comprises the following CDRs:
CDR1: NHAMA (SEQ ID NO: 10);
CDR2: YISYDGDNTYYRDSVKG (SEQ ID NO: 2);
and
CDR3: GGNSGYD (SEQ ID NO: 3);
and
the LCVR comprises the following CDRs:
CDR1: KGSQNVNKFLN (SEQ ID NO: 12);
CDR2: GTNSLQT (SEQ ID NO: 5);
and
CDR3: QQYTSWPWT (SEQ ID NO: 13).

29. The isolated antibody or antigen-binding fragment thereof according to claim 20, being a bispecific antibody.

30. A fusion protein comprising the isolated antibody or the antigen-binding fragment thereof according to claim 20.

31. A nucleic acid encoding the isolated antibody or antigen-binding fragment thereof according to claim 20 or a fusion protein comprising said isolated antibody or antigen-binding fragment thereof.

32. An expression vector comprising the nucleic acid according to claim 31.

33. The isolated antibody or antigen-binding fragment thereof according to claim 20, wherein said antibody or antigen-binding fragment mediates antibody dependent cellular cytotoxicity, complement dependent cytotoxicity or antibody-dependent phagocytosis.

34. A pharmaceutical composition comprising the isolated antibody or antigen-binding fragment thereof according to claim 20 or a fusion protein comprising said isolated antibody or antigen-binding fragment thereof, and at least one pharmaceutically acceptable excipient.

35. A method for treating a cancer or an infectious disease in a subject in need thereof, comprising administering to the subject
the isolated antibody or antigen-binding fragment thereof according to claim 20, or
a fusion protein comprising said isolated antibody or antigen-binding fragment thereof, or
a pharmaceutical composition comprising said isolated antibody or antigen-binding fragment thereof and at least one pharmaceutically acceptable excipient, optionally wherein said isolated antibody or antigen-binding fragment thereof or said fusion protein is administered in combination with an immunotherapy.

36. A method of inducing specific lysis of CD25 positive cells without inhibiting IL-2 signaling in T-cells, the method comprising the step of administering to a subject:
a therapeutically effective amount of the isolated antibody or antigen-binding fragment according to claim 20, or
a therapeutically effective amount of a fusion protein comprising said isolated antibody or antigen-binding fragment thereof, or
a therapeutically effective amount of a pharmaceutical composition comprising said isolated antibody or antigen-binding fragment thereof and at least one pharmaceutically acceptable excipient.

37. The method according to claim 36, wherein the subject is receiving or has received an immunotherapy.

38. A method comprising the step of administering to a subject an immunotherapy, wherein the subject has received or is receiving
a therapeutically effective amount of the isolated antibody or antigen-binding fragment according to claim 20, or
a therapeutically effective amount of a fusion protein comprising said isolated antibody or antigen-binding fragment thereof, or
a therapeutically effective amount of a pharmaceutical composition comprising said isolated antibody or antigen-binding fragment thereof and at least one pharmaceutically acceptable excipient.

39. The method according to claim 38, wherein the therapeutically effective amount is an amount effective to induce specific lysis of CD25 positive cells without inhibiting IL-2 signaling in T-cells.
